SEQUENCE LISTING (12 sequences)

SEQ ID NO: 1 - DNA, 43 bp, Artificial Sequence
  synthetic sequence
gatgtcctacagcgagagattggggagcagacaaagaggtaaa

SEQ ID NO: 2 - DNA, 42 bp, Artificial Sequence
  synthetic sequence
tcccggtttgtactgctaatgtggacaccttgtcgtgttatg

SEQ ID NO: 3 - DNA, 43 bp, Artificial Sequence
  synthetic sequence
gtttgcggctctgaaagaatgcactgttgatccggttcctatt

SEQ ID NO: 4 - DNA, 43 bp, Artificial Sequence
  synthetic sequence
gacgacctgtgggaagatattgcaggaggtagggactgtatga

SEQ ID NO: 5 - DNA, 44 bp, Artificial Sequence
  synthetic sequence
gaggaactgacagtggtacttggggaaggataaagcctggttag

SEQ ID NO: 6 - DNA, 42 bp, Artificial Sequence
  synthetic sequence
ctgtgggttggtggacattagagaggacttcatcggcaatag

SEQ ID NO: 7 - DNA, 41 bp, Artificial Sequence
  synthetic sequence
gggtgtgaaccacgagaaatagggtctgggatggaaattgt

SEQ ID NO: 8 - DNA, 40 bp, Artificial Sequence
  synthetic sequence
cagcaagtgggtggactattatcagtgggcacaagatgag

SEQ ID NO: 9 - DNA, 44 bp, Artificial Sequence
  synthetic sequence
gattgactcccagacgcattaccagtcggstgccttctgattac

SEQ ID NO: 10 - DNA, 38 bp, Artificial Sequence
  synthetic sequence
cgagactgcacggattgttcatcttgcaggtggctctt

SEQ ID NO: 11 - DNA, 40 bp, Artificial Sequence
  synthetic sequence
aggaaggtgggtctgaatctaggaaggtgggtctgaatct

SEQ ID NO: 12 - DNA, 44 bp, Artificial Sequence
  synthetic sequence
agtgtgaggttgatgccttattctctggtccggttagttctttc

(19) United States
(12) Patent Application Publication
(10) Pub. No.: US 2022/0362548 A1
(43) Pub. Date: Nov. 17, 2022

(54) DEVICE AND METHODS FOR TISSUE MOLECULAR PROFILING USING ELECTROPORATION BASED MOLECULAR EXTRACTION

(71) Applicant: Ramot at Tel-Aviv University Ltd., Tel Aviv (IL)

(72) Inventors: Alexander GOLBERG, Tel Aviv (IL); Zohar YAKHINI, Tel Aviv (IL); Julia SHEVIRYOV, Tel Aviv (IL)

(21) Appl. No.: 17/628,148

(22) PCT Filed: Jul. 18, 2020
(86) PCT No.: PCT/IL2020/050804

§ 371 Date: Jan. 18, 2022

Related U.S. Application Data
(60) Provisional application No. 62/875,632, filed on Jul. 18, 2019.

(51) Int. Cl. A61N 1/32; A61B 10/02; G01N 33/68

(52) U.S. Cl. CPC A61N 1/327; A61B 10/0283; G01N 33/68

(57) ABSTRACT

Methods and devices obtain cellular-component, e.g., proteins, RNA, DNA, metabolites, and combinations of these, from a solid tissue in-vivo using electroporation, and subsequently profile such tissue either inside or outside the subject's body. A method for determining if a solid tissue of a subject includes a benign or malignant tumor, or if a space occupying lesion (SOL) within the solid tissue is malignant or benign, includes placing at least one electroporation-electrode within the solid tissue, or within the SOL or in proximity thereto; applying pulsed electric field (PEF) via the at least one electroporation-electrode to thereby induce permeabilization of cells of the solid tissue or the SOL, and consequently release of at least one cellular-component therefrom to an extracellular matrix between and surrounding the cells; extracting the at least one cellular-component from the extracellular matrix.

### FIELD OF THE INVENTION

The present invention relates to devices and methods for obtaining molecules from a solid tissue using electroporation in-vivo or ex-vivo, and profiling such tissue thereafter.

### BACKGROUND

Personalized medicine is the optimization of care on an individual basis. Personalized medicine, based on molecular profiles of tumors and other tissues, has greatly developed over recent decades. In cancer therapy and care, a clear potential in several cases was demonstrated for the personalized approach as compared to traditional therapies. A critical component of a successful therapy tailoring for a subject is a careful diagnosis. An important component of molecular diagnoses in disease tissues, including tumors, is the profiling of DNA, RNA, proteins, metabolites, or any combination thereof, to identify molecular biomarkers that are predictive of subject response. To enable disease profiling, current methods use tissue biopsy, which involves resection of a small tissue sample, a procedure which leads to, e.g., localized tissue injury, bleeding, inflammation, neural damage, fracture, and stress, increasing the potential for tumor growth and metastasis. The impact of this stress on the tissue behavior is not well understood. In addition, only a few biopsies can be performed at a time, limiting the spatial mapping of the sampled site. Some authors even concluded that due to tumor heterogeneity, information from a single biopsy is not sufficient for guiding treatment decisions.

It was recently determined that the current technology's limited support for characterizing tumor molecular heterogeneity is a major limitation of the personalized medicine approach in cancer. Significant genomic evolution that often occurs during cancer progression, creating variability within primary tumors as well as between the primary tumors and metastases. Indeed, recent studies show that a positive result (both successful biopsy and molecular characterization) appear to reliably indicate the presence of a high-risk disease. However, a negative result does not reliably rule out the presence of high-risk disease also because a harvested tissue sample did not capture the most lethal clone of a given tumor (Tosoian et al., 2017). Although improvement of the molecular characterization increased remarkably in the recent decade because of the entrance of new high-resolution sequencing and bioinformatics methods, these technologies remain limited by tissue sampling methods. Thus, tissue sampling remains a curtail limitation to the ability to accurately tailor the therapy to subjects, and therefore, new approaches to molecularly probe and characterize several regions in the tumor are called for.

Electroporation-based technologies have been successfully used to non-thermal irreversible and reversibly change permeabilization of the cell membrane in-vivo, enabling a wide set of applications ranging from tumor ablation to targeted molecules delivery to tissues. Protocols for targeted delivery of electric field to tissues to induce focused electroporation at a predetermined region in organs were previously developed. More recently, it was shown that electroporation technologies selectively extract proteins and ash from biomass. Although electroporation has been used to deliver molecules to tissues and to ablate multiple tumors and metastasis, to the best of our knowledge it has not been proposed to extract molecules for tissue profiling, including tumors.

Accordingly, a need exists for an improved tissue profiling for the identification and evaluation of a cancerous tumor in order to enable precise therapies tailoring. The present invention addresses all the above problems and more, and provides a novel approach for tissue sampling with molecular biopsy using electroporation.

### SUMMARY OF INVENTION

The present invention generally provides a method for determining a cellular-components' profile of a solid tissue of a subject, i.e., a profile of proteins, RNA, DNA, and/or metabolites characterizing said solid tissue, as means for identifying or characterizing abnormality of, or within, said tissue, or a disease state of the subject, e.g., at a remote tissue thereof. The method disclosed is thus useful for differentiating between a normal and a diseased tissue, e.g., a tumor, and furthermore for determining heterogeneity of said tissue. Specifically, said method comprises: (i) placing at least one electroporation-electrode within said solid tissue, or in proximity thereto; (ii) applying a pulsed electric field (PEF) via said at least one electroporation-electrode to induce permeabilization of cells of said solid tissue, and consequently release of at least one cellular-component therefrom to an extracellular matrix between and surrounding said cells; (iii) extracting said at least one cellular-component from said extracellular matrix; and (iv) identifying/analyzing the at least one cellular-component extracted so as to identify/determine abnormality of, or within, said solid tissue, e.g., the presence and type of a tumor within said tissue, or the presence of a disease state of the subject.

In one specific aspect, the invention provides a method for determining if a solid tissue of a subject comprises a benign or malignant tumor, or if a space occupying lesion (SOL) within said solid tissue is malignant or benign, said method comprising: (i) placing at least one electroporation-electrode within said solid tissue, or within said SOL or in proximity thereto; (ii) applying a PEF via said at least one electroporation-electrode to thereby induce permeabilization of cells of said solid tissue or said SOL, and consequently release of at least one cellular-component therefrom to an extracellular matrix between and surrounding said cells; (iii) extracting said at least one cellular-component from said extracellular matrix; and (iv) identifying/analyzing the at least one cellular-component extracted so as to identify/determine the presence and type of the tumor within said solid tissue or determine if said SOL is malignant or benign.

As disclosed herein, identification/analysis of the at least one cellular-component extracted in step (iv), so as to identify/determine (a) abnormality of, or within, said solid tissue, or the presence of a disease state of the subject; or (b) the presence and type of the tumor within said solid tissue or determine if said SOL is malignant or benign, may be carried out either within said at least one electroporation-electrode, i.e., in-vivo, or outside the subject's body (in-vitro), e.g., after removal of said at least one electroporation-electrode.

The present invention thus generally further relates to a method for determining a cellular-components' profile of a solid tissue of a subject, i.e., a profile of proteins, RNA, DNA, and/or metabolites characterizing said tissue, as means for identifying or characterizing abnormality of, or within, said tissue, or a disease state of the subject, e.g., at a remote tissue thereof, said method comprising analyzing/identifying in-vitro at least one cellular-component extracted from cells of said solid tissue, characterized in that said at least one cellular-component has been extracted from said cells in-vivo, by applying a PEF within said solid tissue or in proximity thereto, and consequently releasing said at least one cellular-component therefrom to an extracellular matrix between and surrounding said cells.

In a second specific aspect, the invention thus relates to a method for determining if a solid tissue of a subject comprises a benign or malignant tumor, or if a SOL within said solid tissue is malignant or benign, said method comprising analyzing/identifying in-vitro at least one cellular-component extracted from cells of said solid tissue or SOL, characterized in that said at least one cellular-component has been extracted from said cells in-vivo, by applying a PEF within said solid tissue, or within said SOL or in proximity thereto, and consequently releasing said at least one cellular-component therefrom to an extracellular matrix between and surrounding said cells.

In a third aspect, the present invention provides a device for the extraction of at least one cellular-component from cells of a solid tissue of a subject and/or from cells of a SOL within said solid tissue, for determining (a) a cellular-components' profile of said tissue, i.e., a profile of proteins, RNA, DNA, and/or metabolites characterizing said tissue, as means for identifying or characterizing abnormality of, or within, said tissue, or a disease state of the subject; or (b) if said solid tissue comprises a benign or malignant tumor, or if said SOL is malignant or benign, said device comprising: (i) at least one electroporation-electrode designed to be associated with an electric generator, and to generate a PEF; and (ii) a cellular-components extraction-element, wherein upon introducing said at least one electroporation-electrode into said solid tissue, or into said SOL or in proximity thereto, and applying a PEF, said PEF induces permeabilization of said cells and consequently said at least one cellular-component exits to an extracellular matrix between and surrounding said cells, and is then extracted by said extraction-element.

### BRIEF DESCRIPTION OF THE DRAWINGS

FIGS. 1A-1F illustrate a protocol for molecular harvesting using electroporation from normal liver and kidney in mouse: FIG. 1A is a schematic protocol; FIG. 1B shows the differential expression of genes detected with RNA extracted with electroporation molecular harvesting in mouse liver and kidney (n=6); FIG. 1C is a histogramm of PEF extracted kidney proteins with iBAQ>10⁷; FIG. 1D is a histogramm of PEF extracted liver proteins with iBAQ>10⁷; FIGS. 1E-1F are skewness and kurtosis plots of MW from kidney and liver, respectively.

FIG. 2 is an annotation of identified proteins to processes. The annotation was done on all identified proteins by GOrilla (Eden et al., 2009) using a ranked by the LFQ_liver-LFQ_Kidney list.

FIGS. 3A-3C are pictures of liver tissue: FIG. 3A is a digital image of an excised liver with HepG2 tumor; FIG. 3B is an image of hematoxylin and eosin (H&E) staining of the tumor area; and FIG. 3C is an image of H&E of the normal liver area.

FIGS. 4A-4B illustrate a protocol for molecular harvesting using electroporation from normal liver and HepG2 tumor model in mouse: FIG. 4A is a schematic protocol; and FIG. 4B shows the differential expression of genes detected with RNA extracted with electroporation molecular harvesting in mouse liver and the HepG2 tumor (n=6).

FIG. 5 is an annotation of identified proteins to processes. The annotation was done on all identified proteins by GOrilla using a ranked by the LFQ_tumor-LFQ_liver list.

FIG. 6 is schematics of liquid harvesting from a tissue using only a liquid phase.

FIG. 7 is a schematic description of a harvesting needle according to some embodiments of the invention.

FIG. 8 is a schematic design of a needle electroporation-electrode with opening head according to some embodiments of the invention.

FIG. 9 is schematics of liquid harvesting from a tissue using an adsorbing pad/coating located on the electroporation-electrode.

FIG. 10 is an illustration of placing two electroporation-electrodes within a solid tissue.

FIGS. 11A-11D illustrate an in-vivo procedure for molecular harvesting using e-biopsy with electroporation: FIG. 11A is a schematic illustration of the procedure; FIGS. 11B-11D are images of the e-biopsy procedure showing the needle insertion into the tumor and normal breast (FIG. 11B); the samples locations—2 samples were taken from center, middle and periphery (FIG. 11C); and the areas from which the control samples were taken for proteins extraction using standard lysis buffer (FIG. 11D).

FIG. 12 is a graph showing spearman values of a correlation between duplicate sampling of 4782 proteins by e-biopsy from peripheral, middle and center of the 4T1 tumor in 5 mice in-vivo.

FIG. 13 is a scatter plot of in-vivo e-biopsy vs. Lysis buffer extraction of 4782 proteins ex-vivo in peripheral, middle and center locations of 4T1 tumors in 5 animals. Average values for duplicates of e-biopsy samples for each location are shown.

FIGS. 14A-14C are GoRilla of differential expression of: C vs. NB (FIG. 14A); M vs. NB (FIG. 14B); and P vs. NB (FIG. 14C). FIGS. 14D-14F are overabundance plot of differential expression of: C vs. NB (FIG. 14D); M vs. NB (FIG. 14E); and P vs. NB (FIG. 14F). Total five mice and 4782 per sample analyzed.

FIGS. 15A-15C are GoRilla of differential expression of intratumor proteome heterogeneity of: C vs. P (FIG. 15A); C vs. M (FIG. 15B); and M vs. P (FIG. 15C). FIGS. 15D-15F are overabundance plot of differential expression of: C vs. P (FIG. 15D); C vs. M (FIG. 15E); and M vs. P (FIG. 15F).

### DETAILED DESCRIPTION

Molecular extraction is a starting point in any molecular diagnostic assay. Relative procedures include tissue disruption, cell lysis, sample pre-fractionation, and separation. Although chemical, enzymatic and mechanical methods, including grinding, shearing, beating, and shocking for tissue permeabilization to support molecular extraction are well developed, the extraction of molecules at the point of care is still very challenging. In addition, most of the current methods are very low-throughput, require individual sample manipulation and are not suitable for rapid extractions. The latter is often required when the sample is sensitive and degrades rapidly.

To address these challenges, electric fields have been investigated in the recent decade for enhancing molecular extraction. High-voltage, pulsed electric fields that lead to tissue electroporation is a specific example of these emerging technologies based on electric fields. Previous works already showed use of electroporation for extracting genomic DNA, RNA, and proteins from cells and tissues ex-vivo. However, there is no work that reported on biomolecules extraction from tissues that support differentiation expression analysis, as shown in this work.

The present invention provides electroporation-biopsy (e-biopsy) procedure protocols to obtain molecular profiles of cellular components, e.g., RNA and proteins, obtained through this procedure. In particular, it is shown that e-biopsy extraction of RNA and proteins from HepG2 liver tumor in mice, normal mice liver and normal mice kidney are tissue specific. This new procedure is substantively different from known needle or liquid biopsy tissue characterization, and is expected to overcome various problems of sampling for diagnostics and, thus, enable a new type of diagnostic approach by creating tissue molecular profiling.

E-biopsy for tissue characterization is substantially different from needle or other excision biopsies (with the associated risks as described above), as well as from liquid biopsy (which only sees an average profile and cannot provide sub-clonal information). The present approach, when used in combination with in-situ electroporation-electrodes, provides access to molecular markers from volumes of tissues larger than the used needles, thus expanding the opportunity for capturing clones variations. Furthermore, due to its minimally invasive nature, it leads to enabling multiple sampling and thereby high resolution spatial molecular cartography of tissues.

Accordingly, the present invention provides a method for extracting cellular components, e.g., proteins, RNA, DNA, and/or metabolites, from cells of a solid tissue—either in-vivo or ex-vivo—and using same for determining a cellular-components' profile of said tissue as means for identifying or characterizing: (a) abnormality of, or within, said tissue; (b) a disease state of the subject, e.g., at a tissue other than that directly tested; or (c) presence of a heterogeneity within the tested tissue. Accordingly, the method can be used to differentiate between a normal and a diseased tissue, e.g., a tumor, and furthermore to determine molecular heterogeneity of such a diseased tissue. The method is based on the extraction of the cellular components from cells of the tested tissue using e-biopsy, and comprises: (i) placing at least one electroporation-electrode within said solid tissue, or in proximity thereto; (ii) applying a PEF via said at least one electroporation-electrode to induce permeabilization of cells of said solid tissue, and consequently release of at least one cellular-component therefrom to an extracellular matrix between and surrounding said cells; (iii) extracting said at least one cellular-component from said extracellular matrix; and (iv) identifying/analyzing the at least one cellular-component extracted so as to identify/determine the presence and type of abnormality within said solid tissue or identify/determine the presence of a disease state of the subject.

In a specific such aspect, the present invention provides a method as defined above, for determining if a solid tissue of a subject comprises a malignancy, or if a SOL within such solid tissue is malignant, i.e., for determining if said solid tissue comprises a benign or malignant tumor, or if said SOL is malignant or benign.

The term “heterogeneity” as used herein, also known as “hetergenecity”, refers to a non-homogeneous solid tissue, i.e., a solid tissue comprising different malignant clonal populations or both benign and malignant tumor populations. It also refers to the presence of a malignant tumor population that originated from a different/variant tissue (as a result of metastases).

In certain embodiments, the methods of the invention further allow for determining a more accurate location of possibly present tumor populations within a broad region of a tissue in the subject's body.

The term “subject” as used herein refers to any mammal, e g, a human, non-human primate, horse, ferret, dog, cat, cow, and goat. In a preferred embodiment, the term “subject” denotes a human, i.e., an individual.

The method specifically disclosed hereinabove comprises the steps of: (i) placing at least one electroporation-electrode within a solid tissue, or within a SOL within said solid tissue or in proximity thereto, within a subject's body; (ii) applying a PEF via the at least one electroporation-electrode to thereby induce permeabilization of cells of said solid tissue or said SOL, and consequently release of at least one component of molecular content therefrom to the extracellular matrix between and surrounding said cells; (iii) extracting the at least one cellular-component from the extracellular matrix; and (iv) identifying/analyzing the at least one cellular-component extracted so as to identify/determine the presence and type of a tumor within the solid tissue or determine if the SOL is malignant or benign, or to determine the presence of molecular markers in the probed location.

According to the present invention, identification/analysis of the at least one cellular-component extracted in step (iv) may be carried out in-vivo, in-vitro, i.e., after removal of said at least one electroporation-electrode, or both in-vivo and in-vitro.

In certain embodiments, identification/analysis of the at least one cellular-component extracted is carried out in-vivo, i.e., step (iii) is extracting the at least one cellular-component into at least one of the at least one electroporation-electrode and step (iv) is carried out within said electroporation-electrode, e.g., by pulse amperometic analysis.

In alternative embodiments, identification/analysis of the at least one cellular-component extracted is carried out in-vitro, i.e., step (iv) is carried-out outside the subject's body, by any suitable technique. In specific such embodiments, the method disclosed herein further comprises a step of removing the at least one electroporation-electrode after step (iii) and prior to step (iv).

In further alternative embodiments, identification/analysis of the at least one cellular-component extracted is carried out partially in-vivo and partially in-vitro, i.e., step (iv) is carried out partially within said electroporation-electrode, e.g., by pulse amperometic analysis; and partially outside the subject's body, by any suitable technique, e.g., after removing the at least one electroporation-electrode after step (iii).

In certain embodiments, the method disclosed herein further comprises a preliminary step(s) of obtaining medical imaging-based location's data of the solid tissue and/or of the SOL. In specific embodiments, the medical imaging is MRI, CT, etc. In further embodiments, particularly if no SOL is observed, other preliminary steps, such as blood tests, are performed in order to evaluate whether the solid tissue is suspected of having a malignancy.

In certain embodiments of the method according to any of the embodiments above, the step of placing the at least one electroporation-electrode within the solid tissue, or within said SOL or in proximity thereto, is carried out under real-time imaging, such as CT, MRI, ultrasound, or impedance measurement.

PEF treatment is a process consisting of applying short microsecond pulses of high voltage at high frequency, leading to biological tissue permeabilization. The term “pulsed electric field (PEF)” as used herein thus refers to the application of a pulsed electric field characterized by specific voltage, electric field strength, pulse duration, number of pulses, and pulses frequency. Although the exact mechanism of biological tissue permeabilization by PEF is not fully understood, the current theory suggests that the membrane permeabilization is achieved through the formation of aqueous pores on the cell membrane, a phenomenon known as electroporation.

In certain embodiments of the method according to any of the embodiments above, the PEF is characterized by (i) pulse number of from 1 to about 10,000, e.g., from 1 to about 500, from 500 to about 1000, from about 1000 to about 2000, from about 2000 to about 3000, from about 2000 to about 4000, from about 4000 to about 5000, from about 5000 to about 6000, from about 6000 to about 7000, from about 7000 to about 8000, from about 8000 to about 9000, or from about 9000 to about 10000; (ii) pulse duration of from about 50 ns to about 10 ms, e.g., from about 50 ns to about 500 ns, from about 500 ns to about 1 ms, from about 1 ms to about 2 ms, from about 2 ms to about 3 ms, from about 3 ms to about 4 ms, from about 4 ms to about 5 ms, from about 5 ms to about 6 ms, from about 6 ms to about 7 ms, from about 7 ms to about 8 ms, from about 8 ms to about 9 ms, or from about 9 ms to about 10 ms; (iii) electric field strength of about 0.1 to about 100 kV/cm, e.g., about 0.1 to about 0.5 kV/cm, about 0.5 to about 1 kV/cm, about 1 to about 5 kV/cm, about 5 to about 10 kV/cm, about 10 to about 20 kV/cm, about 20 to about 30 kV/cm, about 30 to about 40 kV/cm, about 40 to about 50 kV/cm, about 50 to about 60 kV/cm, about 60 to about 70 kV/cm, about 70 to about 80 kV/cm, about 80 to about 90 kV/cm, or about 90 to about 100 kV/cm; and (iv) pulse frequency of from 0.1 to about 10000 Hz, e.g., from 0.1 to about 10 Hz, from 10 to about 100 Hz, from 100 to about 500 Hz, from 500 to about 1000 Hz, from 1000 to about 2000 Hz, from 2000 to about 3000 Hz, from 3000 to about 4000 Hz, from 4000 to about 5000 Hz, from 5000 to about 6000 Hz, from 6000 to about 7000 Hz, from 7000 to about 8000 Hz, from 8000 to about 9000 Hz, or from 9000 to about 10000 Hz.

As would be clear to any person skilled in the art, the particular characteristics (properties) of the PEF treatment applied, i.e., the combination of particular pulse number, pulse duration, electric field strength and pulse frequency selected, may affect the efficiency of the process, e.g., the electroporation efficiency, and consequently the amount and/or types of cellular-components released from the electroporated cells. The particular characteristics of the PEF treatment applied should thus be selected such that the permeabilization induced and consequently the release of the cellular component(s) would provide a cellular components profile best reflecting the cells of the target solid tissue or SOL.

In certain embodiments of the method according to any of the embodiments above, the at least one cellular-component released from the cells of the solid tissue or SOL is selected from proteins, RNA, DNA, metabolites, or any combination thereof.

In certain embodiments of the method according to any of the embodiments above, steps (ii) and (iii), and optionally step (iv), are repeated several times, each time at a different location/area within the solid tissue and/or the SOL, without removing the at least one electroporation-electrode therefrom, i.e., by advancing and retracting the electrode within the solid tissue or the SOL. In alternative embodiments, the at least one electroporation electrode is removed from the tissue or the SOL and transferred to a different location/area within the solid tissue and/or the SOL. In specific embodiments, the at least one cellular-component that is released into the extracellular matrix at each location/area is kept parted for separate analysis in step (iv). In specific embodiments, step (iv) is repeated only when the analyzing/identifying of the at least one cellular-component is carried out within the electroporation-electrode as defined above. However, if the analyzing/identifying step (iv) is carried outside the electroporation-electrode, i.e., outside the subject's body, step (iv) is not necessarily repeated in conjunctions with steps (ii) and (iii).

In certain embodiments of the method according to any of the embodiments above, the presence of the SOL has been determined and the at least one electroporation-electrode is placed within the SOL or in proximity thereto, such that at least part of the SOL is within the PEF generated/applied in step (ii).

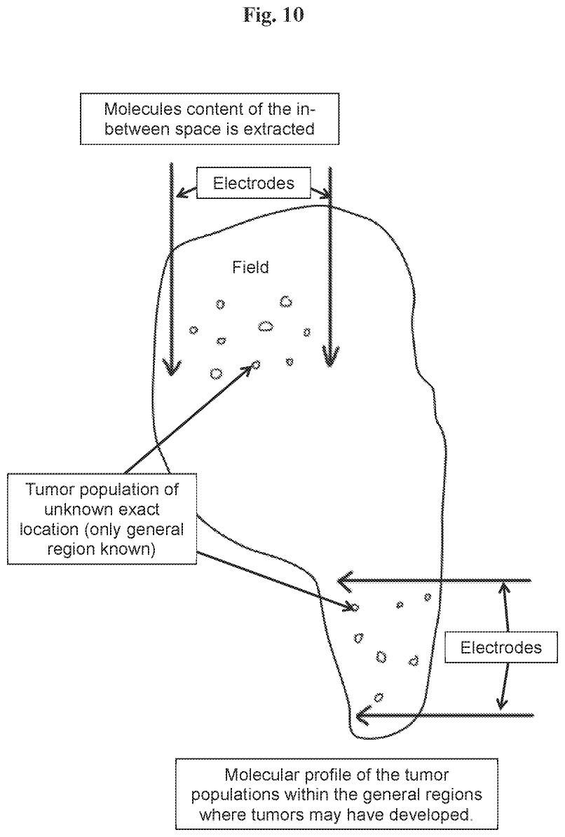

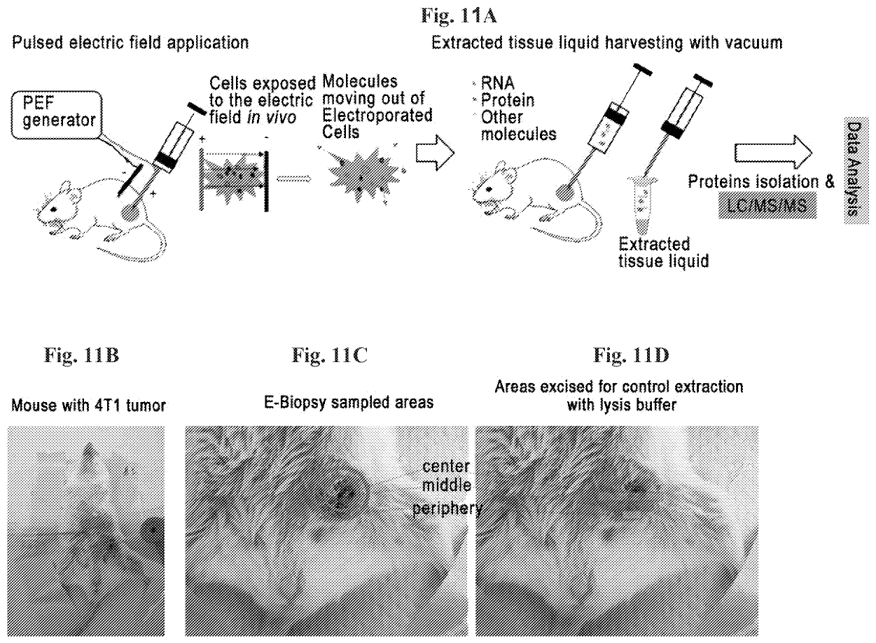

In certain embodiments of the method according to any of the embodiments above, two electroporation-electrodes are used to generate PEF between them. In such a configuration, PEF is generated between the two electroporation-electrodes, which enables release of at least one cellular-component from cells positioned between the two electrodes. This is especially beneficiary when there is no prior knowledge of the location of the malignancy or SOL, or if the size of the malignancy or SOL is too small for accurately positioning a single electroporation-electrode in it or in close proximity thereto. In specific embodiments, both electroporation-electrodes are placed within the solid tissue (see illustration in FIG. 10). In alternative specific embodiments, one electroporation-electrode is placed within the solid tissue (or in proximity thereto), and the other is positioned at a remote location on the body of the subject, e.g., on the skin.

The method disclosed herein, according to any of the embodiments above, enables a physician to obtain molecular profiles from within a subject's organ even without explicitly knowing where and if a tumor or a diseased cell population exists in the organ. This is enabled, in part, by using two or more electroporation-electrodes to release, by electroporation, molecular markers/components from cells positioned between these two or more electroporation-electrodes. The collection and subsequent analysis of these released molecular markers/components give the physician indication of molecular profiles within the probed region.

In certain embodiments of the method according to any of the embodiments above, the at least one electroporation-electrode each independently is designed to enable penetration into the solid tissue, and is: (i) a hollow tube; (ii) a solid rod engulfed in a retentive tube/cannula; or (iii) a solid rod at least partially coated at the area designed to be placed within the tissue with an adhesive material capable of reversibly adsorbing, associating with, and/or linking at least one of the cellular-components. In specific embodiments, the at least one electroporation-electrode is hollow, and the at least one cellular-component released to the extracellular matrix is extracted in step (iii) by suction via said at least one hollow electroporation-electrode. In further specific embodiments, the method further comprises a step of inserting at least one liquid, such as an extraction buffer, water and saline, into the solid tissue or SOL via the at least one hollow electroporation-electrode, and the at least one cellular-component released to the extracellular matrix is extracted in step (iii) by suction together with the liquid via the at least one hollow electroporation-electrode. The liquid may be added at any time point. Accordingly, in certain embodiments, the liquid is added before performing the PEF. In alternative embodiments, the liquid is added after performing the PEF.

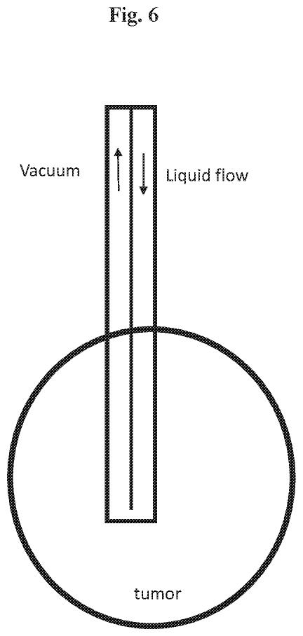

FIG. 6 illustrates liquid harvesting from a tissue using only a liquid phase: extraction liquid (water or any other suitable extraction buffer) flows through the needle into the tissue/tumor. An electric field is delivered through the needle electroporation-electrodes (e.g., the internal electrode is positivly charged and the external electrode is negatively charged). The liquied released from the cells is mixed with the extraction buffer and is sucked outside the body to the outlet, e.g., with vacuum.

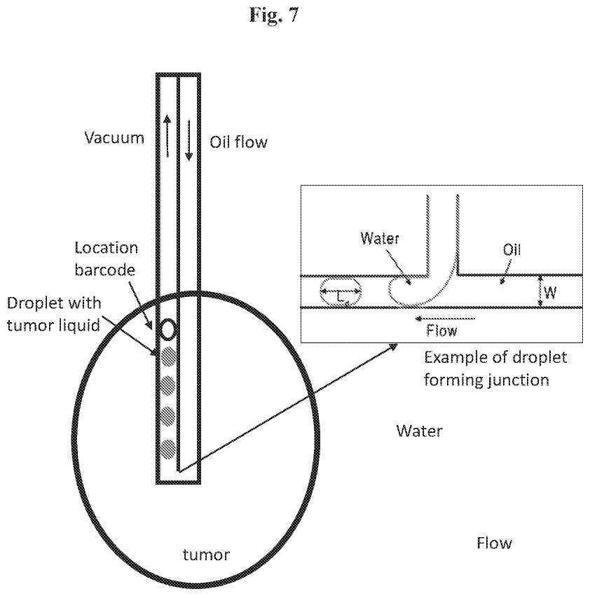

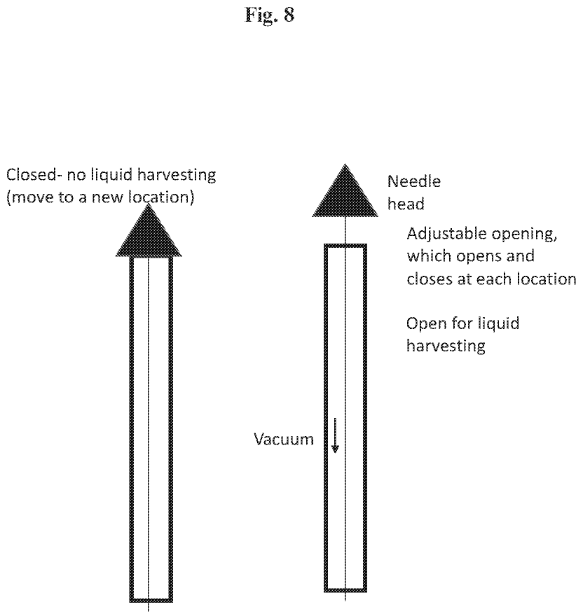

FIG. 7 illustrates liquid harvesting from a tissue using oil according to some embodiments of the invention. The liquid extracted from the cells in the tissue is encapsulated inside droplets, emerged into an oil phase. Labeling and separation between various regions of biopsy is done through the introduction of a barcode inside one or several oil droplets when the needle moves to a new biopsy/harvesting location. The electric field is delivered through the needle electroporation-electrodes (e.g., the internal electrode is positivly charged and the external electrode is negatively charged).

FIG. 8 illustrates a needle electroporation-electrode with an opening head according to some embodiments of the invention. During the location-shift of the needle from one collection point to the other, the needle head is closed. At the diagnozidized/tested location the needle head is opened to enable suction of liquid. Electric fields are delivered and the released liquid is harvested through the opening slot with either extraction buffer, oil and/or directly with vacuum.

In certain embodiments, the addition of the extraction buffer can be carried out at any time point, i.e., (i) after insertion of the electroporation-electrode and prior to the PEF generation; (ii) after the PEF generation, and prior to the extraction of the at least one cellular-component and extracellular matrix; or (iii) simultaneously while extracting the at least one cellular-component and extracellular matrix (i.e., together with the application of PEF).

In further specific embodiments of the above method, the at least one liquid is: (i) an aqueous solution and the at least one cellular-component released to the extracellular matrix is diluted therein for extraction; (ii) an oil and the at least one cellular-component released to the extracellular matrix is encapsulated by the oil to form a micelle that is then extracted by suction; or (iii) an aqueous solution and an oil inserted sequentially in that order, so that the at least one cellular-component released to the extracellular matrix is first diluted in the aqueous solution, and then encapsulated by the oil to form a micelle that is extracted by suction.

In certain embodiments of the method according to the invention, the at least one electroporation-electrode is a solid rod engulfed in a retentive tube/cannula, and the at least one cellular-component released to the extracellular matrix is extracted in step (iii) by suction via the tube/cannula after extraction of the solid rod therefrom once PEF generation is complete. In specific embodiments, the method further comprises a step of inserting at least one liquid, such as an extraction buffer, water and saline, into the solid tissue via the tube/cannula, and the at least one cellular-component released to the extracellular matrix is extracted in step (iii) by suction together with the liquid via the tube/cannula. In further specific embodiments, the at least one liquid is: (i) an aqueous solution and the at least one cellular-component released to the extracellular matrix is diluted therein for extraction; (ii) an oil and the at least one cellular-component released to the extracellular matrix is encapsulated by the oil to form micelles that are extracted by suction; or (iii) an aqueous solution and an oil inserted sequentially in that order, and the at least one cellular-component released to the extracellular matrix is first diluted in the aqueous solution and then encapsulated by the oil to form micelles that are extracted by suction.

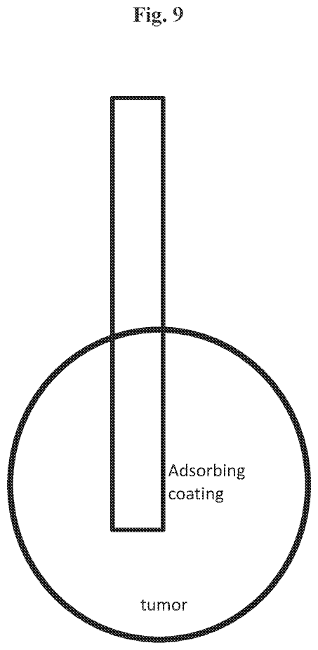

In certain embodiments of the method according to any of the embodiments above, the at least one electroporation-electrode is at least partially coated with an adhesive material capable of reversibly adsorbing, associating with, and/or linking at least one of the cellular-components, and the at least one cellular-component released to the extracellular matrix is analyzed/identified in step (iv) outside the subject's body after removing the at least one electroporation-electrode from the subject's body and releasing the at least one cellular-component therefrom. A particular such electroporation-electrode is a solid rod. FIG. 9 illustrates a needle with an adsorbing coating: after liquid is released/extracted from the cells due to electroporation, the extracted liquid is adsorbed onto the coating and is than taken out (by removing the needle from the tissue) for analysis.

In certain embodiments of the method according to any of the embodiments above, the at least one cellular-component is analyzed/identified in step (iv), by one or more suitable identical or different methods. Examples of methods that may be used include, e.g., protein sequencing, polymerase chain reaction (PCR), sequencing, microarray, chromatography, and mass spectrometry.

In specific embodiments, the presence of a malignancy within the solid tissue and/or if the SOL is malignant, is determined by the method disclosed herein if at least one of the identified cellular-components is indicative of malignancy. In other specific embodiments, the method of the invention determines the presence of a heterogeneity within the malignancy, i.e. a variance of cell colonies within said malignancy (such information might be highly important when considering potential therapeutic treatments for said malignancy). In further specific embodiments, the malignancy is primary malignancy, secondary malignancy, or semi-malignancy. In yet further specific embodiments, the at least one of the identified cellular-components is indicative of either a primary cancer or a secondary cancer.

In specific embodiments, the presence of a heterogeneity, such as fibrosis, or a benign or malignant tumor within said solid tissue, and/or if said SOL is malignant or benign, is determined by the method disclosed herein according to at least one of said identified cellular-components that are indicative therefor.

As disclosed herein, identification/analysis of the at least one cellular-component extracted in step (iv), so as to identify/determine (a) abnormality of, or within, said solid tissue, or the presence of a disease state of the subject; or (b) the presence and type of the tumor within said solid tissue or determine if said SOL is malignant or benign, may be carried out either within said at least one electroporation-electrode, i.e., in-vivo, or outside the subject's body (in-vitro), e.g., after (but not necessarily immediately after) removal of said at least one electroporation-electrode, or after suction of said at least one cellular-component from the subject's body. In a second specific aspect, the present invention thus relates to a method for determining if a solid tissue of a subject comprises a benign or malignant tumor, or if a SOL within said solid tissue is malignant or benign, said method comprising analyzing/identifying in-vitro at least one cellular-component extracted from cells of said solid tissue or SOL, wherein said at least one cellular-component has been extracted from said cells in-vivo, by applying a PEF within said solid tissue, or within said SOL or in proximity thereto, and consequently releasing said at least one cellular-component therefrom to an extracellular matrix between and surrounding said cells.

In certain embodiments, the at least one cellular-component analyzed/identified in-vitro according to this method has been extracted from said cells in-vivo by: (i) placing at least one electroporation-electrode within said solid tissue, or within said SOL or in proximity thereto; (ii) applying a PEF via said at least one electroporation-electrode to thereby induce permeabilization of said cells, and consequently release of said at least one cellular-component therefrom to an extracellular matrix between and surrounding said cells; and (iii) extracting said at least one cellular-component from said extracellular matrix. It should be understood that the at least one electroporation-electrode utilized in step (i) hereinabove can be of any of the designs/configurations referred to in any one of the embodiments herein, and each one of the steps (i) to (iii) hereinabove can be performed according to any one of the those embodiments.

In a third aspect, the present invention provides a device for the extraction of at least one cellular-component from cells of a solid tissue of a subject and/or from cells of a SOL within the solid tissue, for determining if the solid tissue comprises a benign or malignant tumor, or if said SOL is malignant or benign. In certain embodiments, the device comprises: (i) at least one electroporation-electrode designed to be associated with an electric generator, and to generate a PEF; and (ii) a cellular-components extraction-element, wherein upon introducing the at least one electroporation-electrode into the solid tissue, or into said SOL or in proximity thereto, and applying a PEF, the PEF induces permeabilization of the cells and consequently the at least one cellular-component exits to the extracellular matrix between and surrounding said cells or within the solid tissue or SOL and is then extracted outside the solid tissue or SOL by the extraction-element for analysis.

In certain embodiments, the device of the invention further comprises at least one of: (i) a filtering unit at the extraction-element, i.e., in order to filter the liquid while sucking it from within the tissue; and (ii) a power source (such as a pulse electric current generator) associated with the electroporation-electrode(s).

In certain embodiments of the device of the invention, the electroporation-electrode comprises or is associated with a tissue-penetrating element to enable penetration into the solid tissue and SOL.

In certain embodiments, the device of the invention comprises a single electroporation-electrode that comprises a support-element with a first- and second electrical-conductors mounted thereon for creating PEF within the solid tissue, or said SOL or in proximity thereto.

In specific embodiments of the device according to any of the embodiments above, the device comprises two separate electroporation-electrodes, each comprising a support-element with an electrical-conductor mounted thereon for creating PEF within the solid tissue, or said SOL or in proximity thereto, when a PEF is applied between the two electroporation-electrodes. In specific embodiments, the support-element is made of a dielectric material, and optionally comprises or is associated with a tissue-penetrating element to enable penetration into the solid tissue and the SOL.

In certain embodiments of the device according to any of the embodiments above, the extraction-element is an adhesive material capable of reversibly adsorbing, associating with, and/or linking at least one of the cellular-components, wherein the support-element is at least partially coated with the adhesive material.

In certain embodiments, the device according to any of the embodiments above further comprises or is associated with a suction unit, and optionally further comprises or is associated with a collection vessel (such as a syringe or tube) for holding the extracted cellular elements. In specific embodiments, the electroporation-electrode or the support-element is hollow, and constitutes the extraction-element through which cellular-components can be extracted by suction. In alternative specific embodiments, the extraction-element is a retentive tube/cannula engulfing the support-element, so that after PEF is completed and the electroporation-electrode is withdrawn from within the tube/cannula, at least one cellular-component can be extracted from the extracellular matrix in the solid tissue by suction via the tube/cannula.

In certain embodiments, the above device is associated or is designed to be associated with a liquid reservoir and pump, for inserting/pumping at least one liquid into the solid tissue and/or the SOL via, e.g., the support-element for diluting the cellular-components released to the extracellular matrix, so that they can be extracted by suction together with the liquid via the extraction-element.

In certain embodiments of the device according to any of the embodiments above, the at least one liquid is an aqueous solution and the cellular-components released to the extracellular matrix are diluted therein for extraction. In alternative embodiments the at least one liquid is an oil and at least one of the cellular-components released to the extracellular matrix is encapsulated by the oil to form micelles that are then extracted by suction. In yet further alternative embodiments, the at least one liquid is an aqueous solution and an oil inserted sequentially in that order, so that at least one of the cellular-components released to the extracellular matrix is first diluted in the aqueous solution, and then encapsulated by the oil to form micelles that are extracted by suction.

In certain embodiments, the device according to any of the embodiments above further comprises a closure-element (e.g., cap or valve) designed to allow or prevent passage of liquids via the hollow electroporation-electrode or the tube/cannula (see FIG. 8). This configuration enables to move the electroporation-electrode within the solid tissue and/or the SOL without removing the electroporation-electrode therefrom, i.e., by advancing and retracting the electroporation-electrode within the solid tissue while keeping the hollow electroporation-electrode or the tube/cannula clog-free. This is essential when extracting cellular-components from different locations/areas within the solid tissue and SOL, and maintaining the extracted cellular-components from each location/area parted for separate analysis.

The device disclosed herein, according to any of the embodiments above, may be used for carrying out each one of the methods of the invention as described herein.

The present invention demonstrates that macromolecules harvesting using e-biopsy from normal and cancer tissues followed by assessment of the molecular profiles of RNA and proteins obtained thereby, if feasible. It was further showed that RNA and proteins extracted using e-biopsy from HepG2 liver tumor in mice, normal mice liver and normal mice kidney are tissue-specific suggesting that e-biopsy produces sample(s) that can be used for differential expression analysis.

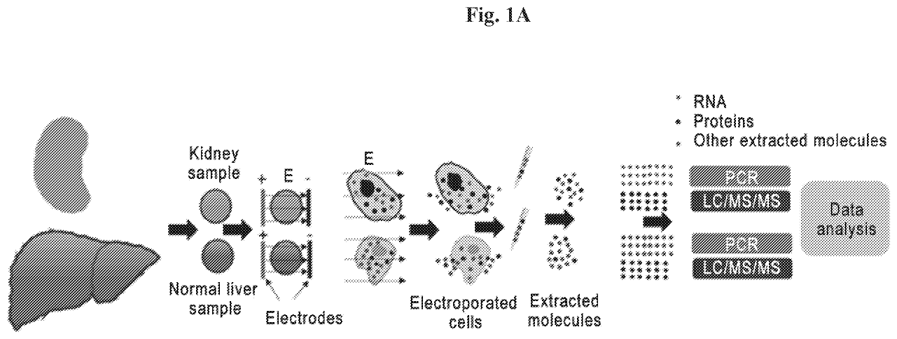

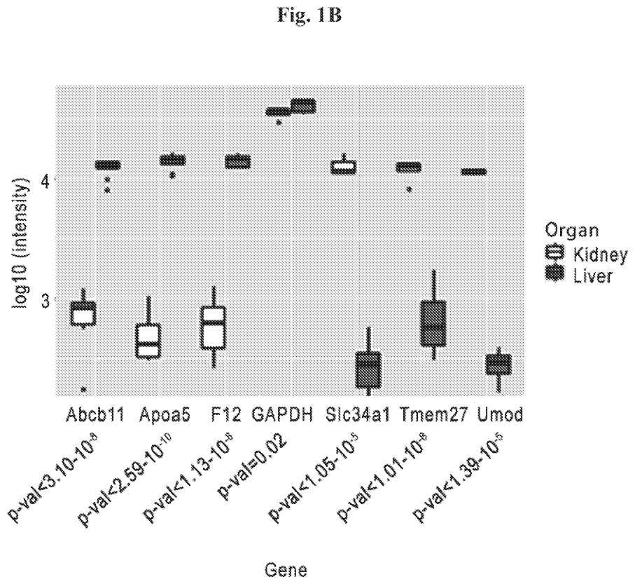

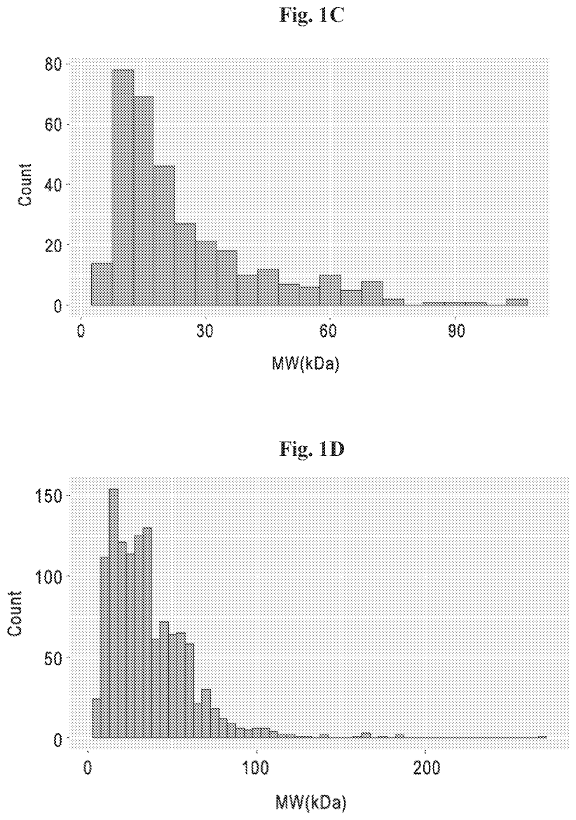

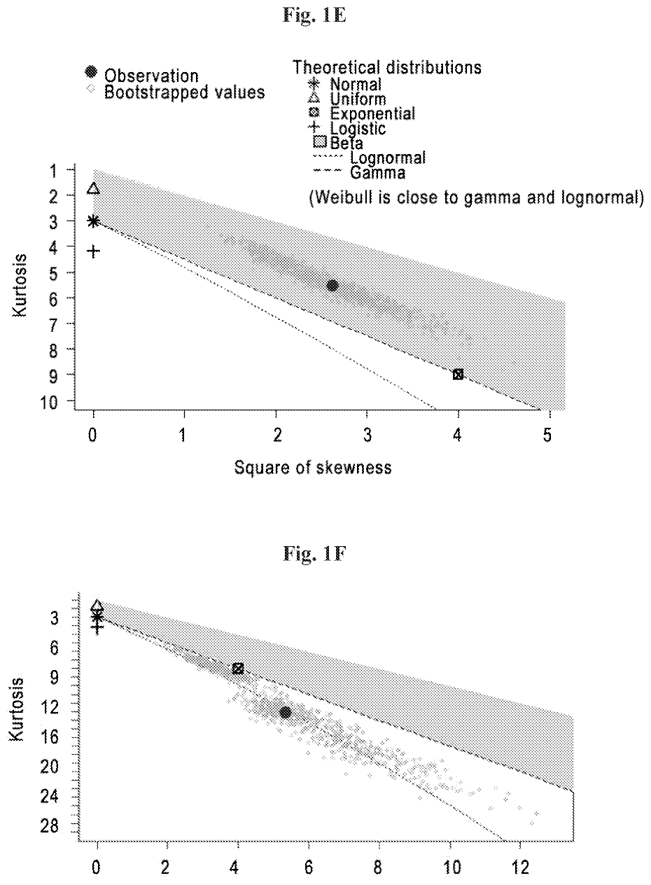

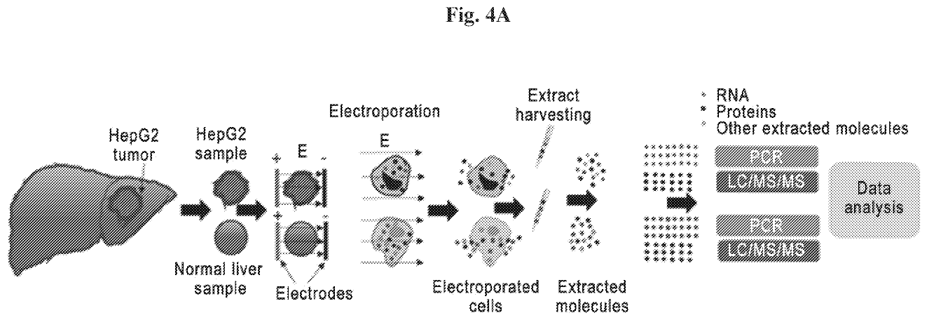

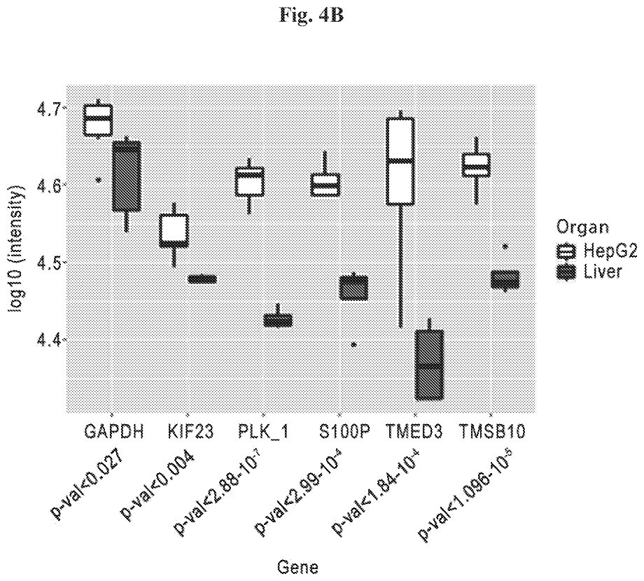

The e-biopsy extract of the kidney contained RNA in higher levels for Tmem27, Umod and Slc34a1 and the e-biopsy extract from the liver contained RNA in higher levels for Apoa5, F12, and Abcb11 (FIG. 1). The present results that show RNA extracted by electroporation, allows for differential expression analysis between the normal liver and normal kidney, which aligns with the literature. These findings were also corroborated by studying the RNA extraction from HepG2 tumor model in mice liver, in which RNA encoding for PLK_1, S100P, TMED3, TMSB10, and KIF23 were significantly higher expressed than RNA for these genes extracted from the normal liver (FIG. 4). Taken together, the above data suggests that e-biopsy extracts tissue-specific RNA and allows for the detection of differential expression between tissues (e.g., kidney and liver) as well as between healthy tissue and tumor.

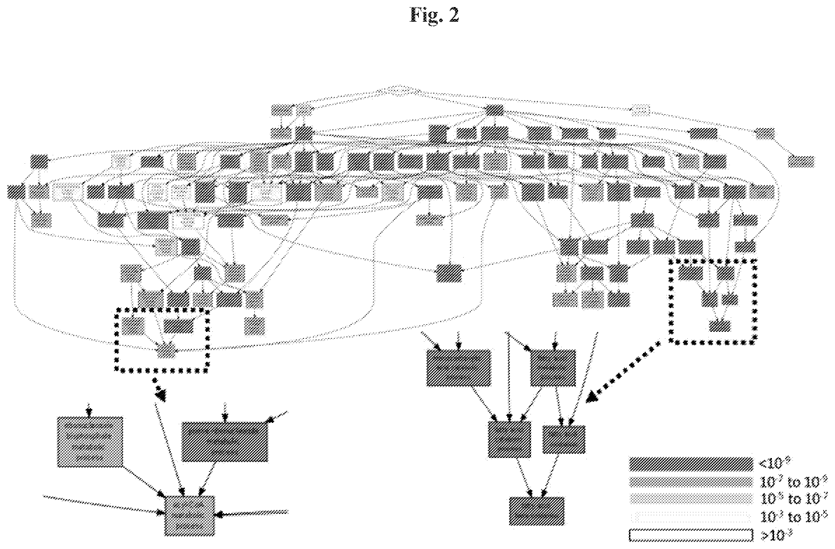

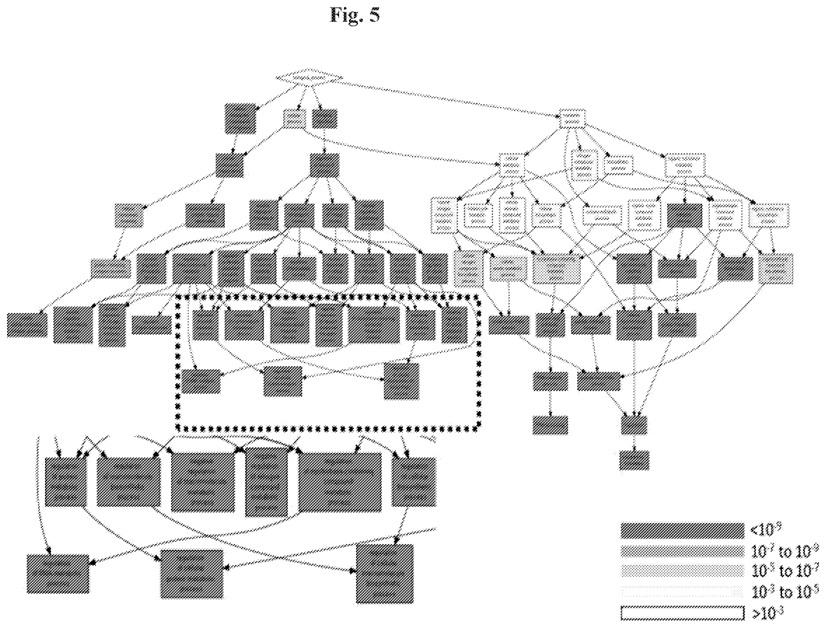

The proteomic analysis of the e-biopsy extract showed that proteins extracted from tissues are tissue-specific (FIG. 2, FIG. 5). Gene Ontology (GO) analysis of the ranked lists of the extracted proteins showed significant differences in process, function, and component associated with proteins extracted from the kidney, liver and HepG2 tumor model in mice liver. The present invention shows that the extracted proteins and RNA are tissue-specific and allow differential expression to be determined in various tissues including tumors. Future studies should determine the properties of the extractable proteins and RNA of various tissues. These properties depend on the tissue structure, using pulsed electric fields protocols and the extraction solvent. The combined knowledge of the physicochemical properties of the extractable protein and RNA, and the structure and chemical properties of the analyzed tissue, could provide new ways for optimizing pulsed electric field parameters such as electric field strength, pulse duration, pulse number, and frequency.

Molecular harvesting with electroporation (e-biopsy) introduced in this application is a new concept for tissue molecular profiling. Although the permeabilization by electroporation is known for delivering molecules to tissues and cells (drugs, vaccines etc.) or to directly kill cells, temporary permeabilization of tissue to facilitate molecular harvesting has not been previously proposed and devices that allow for the harvesting of molecules from tissues do not exist.

Molecular cartography of a tumor is a quantitative, either binary, integer of real valued, annotation of tumor subpopulations, in their defined original positions within a greater tumor location. Intra-tumor heterogeneity may foster tumor evolution and adaptation and hinder current personalized-medicine strategies that depend on results from single tumor-biopsy samples. Furthermore, intra-tumor heterogeneity could lead to the rapid spread of resistant subclones, originally not detected. Molecular cartography provides molecular level information about different sub-regions of the tumor, including differences between the clones that occupy these spaces, which can serve to produce a more accurate predictions and therapeutic recommendations.

Molecular cartography can be at a high resolution—inferred for very small populations within a larger sample or at a lower resolution—inferred for just a few separate regions in a tumor or in 10-20 such regions.

### Examples

### Methods

### Animals

8-week old female Athymic Nude mice weighting ˜20 g were provided by the Science in Action CRO. The animals were housed in cages with access to food and water and libitum and were maintained on a 12 h light/dark cycle in a room temperature of around 21° C. and a relative humidity range of 30 to 70%. All in-vivo experiments were conducted by a professional veterinary.

### In-Vivo Human HepG2 Liver Tumor Model

10⁶ HepG2 cells (50 mL) were directly injected into the mice liver. Four to five weeks after the cells injection, the mice were euthanized with CO₂ and the tissues were immediately harvested for extraction with pulsed electric fields.

### Pulsed Electric Field Application for Biomolecules Extraction Ex-Vivo

First, 250-300 mg of tissue was excised and loaded into electroporation cuvette (BTX electroporation cuvettes plus, 2 mm, Model No. 620, Harvard Apparatus, MA). The cuvette was inserted into custom-made electroporation cuvette holder and connected to the electric field pulse generator (BTX830, Harvard Apparatus, MA). Electroporation was performed using a combination of high-voltage short pulses with low-voltage long pulses as follows: 50 pulses 500V cm⁻¹, 30 μs, 1 Hz, and 50 pulses 50 Vcm⁻¹, 10 ms, delivered at 1 Hz. After the PEF treatment, 300 μl nuclease-free water was added to the cuvette for “juice” dilution and then liquids transferred to 1.5 ml tubes.

RNA Isolation and Amplification from the Pulsed Electric Field Extracted Juice

The total RNA was extracted using water-saturated phenol and 1-Bromo-3-chloropropane (Biological Industries, Beit Haemek Ltd). The cDNA used for PCR was synthesized from total RNA using GoScript™ Reverse Transcription System (Promega Corporation, Madison, Wis., USA).

For the normal tissue differentiation (kidney vs. liver), PCR, 6 pairs of specific primers (Slc34a1, Umod, Tmem27, Apoa5, F12, and Abcb11) were designed according to the mouse transcriptome (Table 1). For gene selection, mouse liver and kidney RNA-seq data was downloaded from Newman et al., 2017 (GEO ID: GSE101657) with five mice per tissue. Normalization and differential expression (DE) analysis were done using DESeq2. A gene was considered to be DE if its corrected p-value<0.01, log 2 (fold-change)>111 and with its average read coverage >100 normalised reads. Selected DE genes were also manually checked to see if their human orthologs are also liver/kidney-specific according to human protein atlas (https://www.proteinatlas.org/)

TABLE 1
Primers used for the mouse liver and mouse kidney differentiation
by the RNA extracted with electroporation

| Gene | | |
|---|---|---|
| Abbreviation | Gene name | Forward/reverse primer |
| Slc34al | Solute carrier family 34 | 5’- GAT GTC CTA CAG CGA GAG ATT G -3’ |
| | (sodium phosphate), | 5’- GGG AGC AGA CAA AGA GGT AAA -3’ |
| | member 1 | |
| Umod | Uromodulin | 5’- TCC CGG TTT GTA CTG CTA ATG -3’ |
| | | 5’ - TGG AC A CCT TGT CGT GTT ATG -3’ |
| Tmem27 | Collectrin, amino acid | 5’- GTT TGC GGC TCT GAA AGA ATG -3’ |
| | transport regulator | 5’ - CAC TGT TGA TCC GGT TCC TAT T -3’ |
| Apoa5 | Apolipoprotein A-V | 5’- GAC GAC CTG TGG GAA GAT ATT G -3’ |
| | | 5’ - CAG GAG GTA GGG ACT GTA TGA -3’ |
| F12 | Coagulation factor XII | 5’- GAG GAA CTG AC A GTG GTA CTT G -3’ |
| | (Hageman factor) | 5’ - GGG AAG GAT AAA GCC TGG TTA G -3’ |
| Abcbll | ATP binding cassette | 5’- CTG TGG GTT GGT GGA CAT TA -3’ |
| | Subfamily B member 11 | 5’ - GAG AGG ACT TCA TCG GCA ATA G -3’ |
| GADPH_ | Glyceraldehyde 3- | 5’- GGG TGT GAA CCA CGA GAA ATA -3’ |
| mouse | phosphatedehydrogenase | 5’ - GGG TCT GGG ATG GAA ATT GT -3’ |

The PCR amplification protocol was 95° C. for 30 s, 40 cycles of 95° C. for 5 s, 55° C. for 10 s, and 72° C. for 30 s. Twenty-seven normal liver and 18 normal kidney samples from 3 mousses were taken for RNA extraction. All samples were collected in fresh conditions and transferred on ice from the surgery room to the laboratory.

For differentiation between tumor and normal liver tissue, 5 pairs of gene-specific primers (PLK1, TMED3, TMSB10, S100P, and KIF23) were designed according to the human transcriptome (Table 2). For gene selection for the analysis, RNA-seq. of help to-cellular carcinoma were downloaded and matched normal samples from TCGA (TCGA LIHC). Normalization and DE analysis were done using DESeq2. A gene was considered as DE, if it's corrected p-value <0.01 and log 2 (fold-change)>111.

TABLE 2
Primers used for the mouse liver and HepG2 kidney differentiation
by the RNA extracted with electroporation

| Gene | Gene name | Forward/reverse primer |
|---|---|---|
| Abbreviation | | |
| PLK1 | Serine/threonine-protein | 5’- CAG CAA GTG GGT GGA CTA TT -3’ |
| | kinase | 5’- ATC AGT GGG CAC AAG ATG AG -3’ |
| TMED3 | Transmembrane p24 | 5’- GAT TGA CTC CCA GAC GCA TTA C -3’ |
| | trafficking protein 3 | 5’- CAG TCG GST GCC TTC TGA TTA C -3’ |
| TMSB10 | Thyomosin beta-10 | 5’- CGA GAC TGC ACG GAT TGT T -3’ |
| | | 5’- CAT CTT GCA GGT GGC TCT T -3’ |
| SIOOP | S100 calcium-binding | 5’- AGG AAG GTG GGT CTG AAT CT -3’ |
| | protein P | 5’- AGG AAG GTG GGT CTG AAT CT -3’ |
| KIF23 | Kinesin-like protein KIF23 | 5’- AGT GTG AGG TTG ATG CCT TAT T -3’ |
| | | 5’- CTC TGG TCC GGT TAG TTC TTT C -3’ |

The cancerous up-regulated genes (the genes with log 2 (fold-change)>111) were further filtered to include only genes that in both HepG2 RNA-seq. data from Solomon et al., 2017, and HepG2 RNA-seq. data from the ENCODE project (Dunham et al., 2012), the expression level is higher than 74% of the expressed genes (reads per kilobase million, RPKM>10 in both Solomon et al. and ENCODE). Using human protein atlas, we manually checked that the selected cancerous up-regulated genes are considered as elevated in cancer but lowly expressed in normal liver.

The up-regulated in normal liver (down-regulated in the cancerous liver) were further filtered to include genes that in both Solomon et al. and in ENCODE HepG2 data, have gene expression that is lower than 75% of the expressed genes (RPKM<0.05 in both Solomon et al. and ENCODE). Using human protein atlas, we manually checked that the selected genes are considered as lowly expressed in cancer and elevated in normal liver.

The PCR amplification protocol was 95° C. for 30 s, 40 cycles of 95° C. for 5 s, 55° C. for 10 s, and 72° C. for 30 s, and the primers used are listed in Table 2.

Seven tumor and 14 normal mouse liver samples from 5 mice were taken for RNA extraction. All samples were collected in fresh conditions and transferred on ice from the surgery room to the laboratory.

The RNA was separated using 1.2% E-Gel electrophoreses system (ThemoFisher, CA). The gel images were captured with ENDURO™ GDS camera (Labnet Inc., NJ). Quantification was done with ImageJ (ver 1.52e, NIH, MA).

Proteins Isolation from the Pulsed Electric Field Extracted Juice

Proteins were isolated from the PEF extract using the protocol of EZ-RNA II kit (Biological Industries, Beit Haemek Ltd). Air-dried protein pellets were taken for proteomic analysis as described below.

Extracted Proteins Identification Quantification with LC-MS/MS

Proteolysis. The samples were brought to 8M urea, 400 mM ammonium bi-carbonate, 10 mM DTT, vortexed, sonicated for 5′ at 90% with 10-10 cycles, and centrifuged. Protein amount was estimated using Bradford readings. 20 ug protein from each sample was reduced 60° C. for 30 min, modified with 37.5 mM iodoacetamide in 400 mM ammonium bicarbonate (in the dark, room temperature for 30 min) and digested in 2M Urea, 100 mM ammonium bicarbonate with modified trypsin (Promega) at a 1:50 enzyme-to-substrate ratio, overnight at 37° C. Additional second digestion with trypsin was done for 4 hours at 37° C.

Mass spectrometry analysis. The tryptic peptides were desalted using C18 tips (Harvard) dried and re-suspended in 0.1% Formic acid. The peptides were resolved by reverse-phase chromatography on 0.075×180-mm fused silica capillaries (J&W) packed with Reprosil reversed phase material (Dr. Maisch GmbH, Germany) The peptides were eluted with linear 180 minutes gradient of 5 to 28% 15 minutes gradient of 28 to 95% and 25 minutes at 95% acetonitrile with 0.1% formic acid in water at flow rates of 0.15 μl/min. Mass spectrometry was performed by Q-Exactive plus mass spectrometer (Thermo) in a positive mode using repetitively full MS scan followed by collision induces dissociation (HCD) of the 10 most dominant ions selected from the first MS scan.

The mass spectrometry data from all the biological repeats were analyzed using the MaxQuant software 1.5.2.8 (Mathias Mann's group) vs. the mouse proteome from the UniProt database with 1% FDR. The data were quantified by label-free analysis using the same software, based on extracted ion currents (XICs) of peptides enabling quantitation from each LC/MS run for each peptide identified in any of the experiments.

The functional groups of the extracted proteins were identified and statistically analyzed using Gene Ontology (GO) analysis with GOrilla, annotating the ranked gene list to the mouse genome.

### Statistical Analysis

Statistical analysis was performed using R-studio, fitdistrplus, ggplot2 and dplyr packages (RStudio: Integrated development environment for R (Version 1.1.383) [Windows]. Boston, Mass.).

RNA and Proteins Differential Expression with e-Biopsy in Mouse Liver and Kidney

FIG. 1A illustrates the protocol for e-biopsy from normal liver and normal kidney. FIG. 1B shows that in the electroporation extracted from kidney, the expression of RNA encoding for Tmem27, Umod and Slc34a1 was significantly higher than that in the liver. Furthermore, Apoa5, F12, and Abcb11 genes were significantly higher in the e-biopsy extracts from the liver than in the extracts from the kidney.

Using semi-quantitative proteomic data, the following parameters were calculated for proteins extracted from liver and kidney: molecular weight (MW), normalized intensity for each sample (LFQ), intensity and normalized within sample intensity (iBAQ). Using these quantitative data, a list of most abundant proteins with iBAQ>10⁷ was selected for further analysis (Table 3). The histogram and density function (FIGS. 1C & 1D) suggested the proteins extracted by e-biopsy have a heavy right tail distribution function. The skewness and kurtosis plots of MW (FIGS. 1E & 1F) suggested that MW has lognormal, gamma or Weibull distributions. The goodness of fit analysis (Table 4 & Table 5) suggests that MW of the most abundant proteins extracted by electroporation is closer to lognormal distribution (smallest statistics for all checked criteria) (Table 6 & Table 7). The parameters and the uncertainty in the parameters (confidence interval) for the lognormal distribution function were determined using bootstrapping.

Interesting, the proteins extracted from the kidney had almost twice lower MW than the proteins extracted from the liver. This can be explained by a different electroporation threshold of cells and by different diffusion properties of properties in these two media.

TABLE 3
Descriptive statistics of molecular weights of proteins
extracted from tissues with electroporation. iBAQ >10⁷

| Tissue | Min | 1st Qu. | Median | Mean | 3^{rd} Qu. | Max |
|---|---|---|---|---|---|---|
| Kidney | 2.79 | 11.99 | 18.01 | 25.21 | 31.53 | 106.25 |
| Liver | 3.81 | 18.38 | 30.53 | 36.38 | 48.35 | 272.43 |
| HepG2 | 3.82 | 18.92 | 32.19 | 40.26 | 50.88 | 394.46 |

TABLE 4
The goodness of fit analysis of highly abundant electroporation
extracted kidney proteins (iBAQ > 10⁷)

| | | |
|---|---|---|
| Weibull | lognormal | gamma |

Goodness-of-fit statistics

| | | | |
|---|---|---|---|
| Kolmogorov-Smirnov statistic | 0.1049928 | 0.06978198 | 0.1079061 |
| Cramer-von Mises statistic | 1.3454622 | 0.43578147 | 1.1439954 |
| Anderson-Darling statistic | 8.1846425 | 2.68808789 | 6.5859668 |

Goodness-of-fit criteria

| | | | |
|---|---|---|---|
| Akaike's Information Criterion | 2776.322 | 2706.840 | 2746.318 |
| Bayesian Information Criterion | 2783.968 | 2714.486 | 2753.965 |

TABLE 5
The goodness of fit analysis of highly abundant electroporation
extracted liver proteins (iBAQ > 10⁷)

| | | |
|---|---|---|
| Weibull | lognormal | gamma |

Goodness-of-fit statistics

| | | | |
|---|---|---|---|
| Kolmogorov-Smirnov statistic | 0.05698024 | 0.0374255 | 0.03459732 |
| Cramer-von Mises statistic | 0.84034789 | 0.4859410 | 0.36004244 |
| Anderson-Darling statistic | 7.69096820 | 2.9875080 | 2.82114915 |

Goodness-of-fit criteria

| | | | |
|---|---|---|---|
| Akaike's Information Criterion | 10940.07 | 10805.36 | 10845.93 |
| Bayesian Information Criterion | 10950.31 | 10815.59 | 10856.16 |

TABLE 6
Parametric bootstrap medians and 95% percentile
CI for lognormal distribution of the MW of
electroporation extracted kidney proteins

| | Median | 2.5% | 97.5% |
|---|---|---|---|
| meanlog | 3.0043648 | 2.9364384 | 3.0735471 |
| sdlog | 0.6527423 | 0.6061976 | 0.7046646 |

TABLE 7
Parametric bootstrap medians and 95% percentile
CI for lognormal distribution of the MW of
electroporation extracted liver proteins

| | Median | 2.5% | 97.5% |
|---|---|---|---|
| meanlog | 3.3893310 | 3.3525484 | 3.4260361 |
| sdlog | 0.6514971 | 0.6241754 | 0.6792361 |

2078 proteins from the kidney and liver were identified using unlabeled proteomic: gene ontology analysis was performed for the associated genes (on the ranked list of differently expressed proteins (Table 8) using GOrilla (Eden et al., 2009), annotating the ranked gene list to the mouse genome. Analysis of the gene onthology by processes showed that small molecule metabolic processes, organic acid metabolic processes, drug metabolic processes, and fatty acid metabolic processes, were higher in the liver than in the kidney (FIG. 2, Table 9).

TABLE 8
Proteins

| | | | | | |
|---|---|---|---|---|---|
| Fabp1 | Cat | Uox | Aspdh | Spr | Dap |
| Prdx1 | Sord | Decr1 | Ftl1 | Cyp2c29 | Uroc1 |
| Cps1 | Hmgcs2 | Hspa8 | Aldh6a1 | Cox6a1 | Lap3 |
| Hspe1 | Aldh1l1 | Haao | Ttr | Uqcrfs1 | Gpd1 |
| Bhmt | Gpx1 | Park7 | Mdh1 | Ephx2 | Cmbl |
| Ass1 | Scp2 | Blvrb | Cox5a | Ak3 | Rps3a |
| Arg1 | Gnmt | Glul | Acaa1a | Pfn1 | Atp5f1 |
| Gstm1 | Atp5a1 | Mpst | Glyat | Als2 | Ttc36 |
| Fah | Ddt | Sardh | Alad | Esd | Hebp1 |
| Aldh1a1 | Abhd14b | Idh1 | Aldh4a1 | Echs1 | Hist1h1d |
| Sod1 | Otc | Pebp1 | Dmgdh | Adh5 | Bdh1 |
| Alb | Ahcy | Gstt1 | Hint1 | Mif | Ctrb1 |
| Acaa2 | Fbp1 | Pcbd1 | Ndufa4 | Timm8b | Rps19 |
| Rgn | Actb | Adk | Mdh2 | Hspa9 | Acads |
| Aldob | Got1 | Cox4i1 | Msra | Asl | Suclg1 |
| Ppia | Atp5j | Mup2 | Pdia3 | Cyb5r3 | 4931406C07Rik |
| Acat1 | P4hb | Sod2 | Cyp2d10 | Inmt | Rrbp1 |
| Etfa | Glud1 | Pgk1 | Psap | Nipsnap1 | Agmat |
| Adh1 | Hgd | Cyp2d26 | Hacl1 | Hnrnpa2b1 | Pgls |
| Gstz1 | Hspa5 | Hadh | Sult1a1 | Aldh9a1 | Hadhb |
| Prdx6 | Aldh2 | Hadha | Etfb | Glo1 | Pgm1 |
| Gsta3 | Akr1c6 | Uox | Hmgcl | Spr | Ftcd |
| Atp5b | Got2 | Decr1 | Mgst1 | Tst | Sdha |
| Tkt | Hspd1 | Cpt2 | Rps14 | Immt | Rpl10a |
| Hint2 | Sec14l2 | Rpn1 | Dbt | Ube2n | Kng1 |
| Agxt | Gstk1 | Ywhag | Ndufc2 | Manf | Mcee |
| Vcp | Etfdh | Ckm | Acsl1 | Nucb1 | Mavs |
| Pah | Hsd11b1 | Dpys | Mt1 | Cox7a2 | Ppib |
| Fasn | Grn | Aifm1 | Ubqln1 | Glod4 | Idi1 |
| Rps15 | Fth1 | Nit1 | Glrx | Slc25a10 | Ube2l3 |
| Aldh7a1 | Pdia4 | Rps3 | Gstt3 | Rdx | Pla2g12b |
| Timm13 | Pipox | Fga | Dld | Psme1 | Chchd10 |
| Suclg2 | Gcdh | Ywhaz | 2210010C04Rik | Ogdh | Snd1 |
| Lypla1 | Sds | Tufm | Dlst | Arhgdia | Fdx1 |
| Ivd | Uqcrh | Eif4a2 | Myh9 | Sec14l4 | Ugt2b1 |
| Sfxn1 | Gpt2 | Psmb2 | Fabp2 | Dhrs4 | Eif4h |
| Atp5c1 | Rps10 | Pck1 | Vapb | Anxa5 | Ctrl |
| Krt76 | Rpl12 | Cela1 | Acy1 | Aadac | Myh4 |
| Nit2 | Acadvl | Cyp2e1 | Ssr4 | Pnpo | Cdv3 |
| Acadl | Shmt1 | Cpb1 | Acot13 | S100a10 | Echdc2 |
| Gc | Hagh | Ndufs6 | Me1 | Cyp2a12 | Idh3a |
| Ak2 | Papss2 | Gstt2 | Reep6 | Rpl18 | Gsta4 |
| Mthfd1 | Uqcrc1 | Rps11 | Hyi | Mrpl12 | Mvp |
| Cstb | Phb | Pabpc1 | Fdps | Ufm1 | Usmg5 |
| Aldh8a1 | Hibadh | Khsrp | Hnrnpk | Prdx3 | Crot |
| Ppa1 | Cryz | Stard10 | Rplp2 | Snx3 | Hnrnpd |
| Rpl6 | C3 | Ech1 | Sar1b | Ufc1 | Nudt7 |
| Hsd17b4 | Sdhb | Slc27a2 | Dstn | Chdh | Ttpa |
| Grhpr | Idh2 | Bckdha | Cyc1 | Nnt | Iqgap2 |
| Khk | Ugp2 | Slc25a13 | Ldhd | Ppif | Amacr |
| Cbr1 | Ces1 | Eef1d | Aco2 | Hpx | Psmc5 |
| Timm9 | Uqcrc2 | Shmt2 | Fkbp2 | Cpa1 | Gclc |
| B2m | Ndrg2 | Prss2 | Cisd1 | Ndufa2 | Phyh |
| Acadm | Taldo1 | Bola1 | Ganab | Mecr | Cela3b |
| Agxt2 | Rps17 | S100a9 | Pnlip | Eea1 | Dlat |
| Pdia6 | Vapa | Rps20 | Akr1c14 | Arpc3 | Psma5 |
| Acsf2 | Ndufv3 | Aldh1a7 | Abhd11 | Rab14 | Adhfe1 |
| Atox1 | Pecr | Galm | Plg | Fgb | Bag3 |
| Aco1 | Pzp | Grpel1 | Rpl30 | Cpox | Gbe1 |
| Bphl | Phb2 | Nedd8 | Tubb2a | Pccb | Mccc2 |
| Ehhadh | Suox | Pgrmc1 | Slc25a3 | Psma3 | Lipa |
| Cyp2f2 | Ctsb | Fmo1 | Hspb1 | Sri | Gvin1 |
| Apoe | Rps21 | Fabp5 | Rpl7 | Pdhb | Gdi2 |
| Tpi1 | Ndufv2 | Ethe1 | Rps23 | Cltc | Sdf2l1 |
| Ctsd | Hdlbp | Ahsg | Glrx5 | Psma7 | Pter |
| Slc25a5 | Cyb5b | Cycs | Ndufv1 | Rpn2 | Gaa |
| Fkbp1a | Cfl2 | Hist1h1e | Asgr1 | Nrn1 | Rbpms2 |
| Tpmt | Pmm2 | Scrn2 | Prdx4 | Plbd2 | Pigr |
| Aldoa | Ndufa12 | Apeh | Dbi | Mcfd2 | Reep5 |
| Scpep1 | Igfbp4 | Ddb1 | Trap1 | Gclm | Ociad2 |
| Coq9 | Hnrnph1 | Top1 | Ddah1 | Bsg | C6 |
| Keg1 | Hsd17b11 | Apoc4 | Pdlim5 | Ewsr1 | Fn1 |
| Pdxk | Ndufa11 | Cnpy2 | Atp5d | Pfdn5 | Ctsz |
| Mtpn | Dnaja1 | Lpp | Txndc17 | Ociad1 | Edf1 |
| Cyp2d22 | Ephx1 | Ugt3a2 | Cfh | Rab10 | Arhgdib |
| Ctsc | Eif1 | Ddx5 | Cndp2 | Snrpd1 | Myh1 |
| Dcxr | Ndufa9 | Atp5j2 | Bpgm | Spcs2 | Sept11 |
| S100a11 | Ppa2 | Ccdc58 | Opa1 | Pygm | Scamp3 |
| Lgals3 | Dynll2 | Ncl | Acp6 | Ndufb3 | Npc2 |
| Eef1a1 | Eif4ebp2 | Ywhah | Apoo | Serpinf2 | Gsn |
| Cdc42 | Dmd | Ehd3 | Snrpd3 | Sh3bgrl | Aga |
| Rnase1 | Snx12 | Clpx | As3mt | Eif6 | Hsbp1 |
| Pdlim1 | Hnrnpf | Sf1 | Eprs | Caprin1 | Iscu |
| Cox7a2l | Eef1b2 | Ugdh | Hmga1 | Aimp1 | Ddi2 |
| Ndufab1 | Mycbp | Arpc1b | Hnrnpa1 | Ppbp | Mrpl49 |
| Bpnt1 | Fau | Fubp1 | Jup | Vtn | Arf5 |
| Cox6b1 | Arpp19 | Capza2 | Hdgf | Mia3 | Pnkd |
| Xylb | Pm20d1 | Hrg | Ndufa5 | Sept9 | Kars |
| Ybx1 | Hp | Tubb5 | Lcp1 | Isg15 | Cmpk1 |
| Acox2 | Lactb2 | Arf4 | Snrpc | Prkar2a | Pcnp |
| Ppp2r1a | Apoc3 | Tpp1 | Pcyt2 | Cnn3 | Dcps |
| Ndufs3 | Fabp4 | Pcbd2 | Mocs2 | Arfgap2 | Apool |
| Psmc6 | Tuba4a | Lmna | L2hgdh | Mtap | Xrcc5 |
| Acad11 | Clta | Crip1 | Pdhx | Nars | Rbbp9 |
| Cap1 | Ndufb8 | Creld2 | Acsm5 | Cast | Sult1d1 |
| Dazap1 | Lrrc59 | Stbd1 | Lgals1 | Pnpla8 | Cyp4a12a |
| S100a13 | Psmd4 | St13 | Tmed10 | Apom | Stat3 |
| Gss | Pdcd6ip | Iigp1 | Msn | Carhsp1 | Dsp |
| Clps | Cct2 | Coasy | Dnaja3 | Tbca | Idh3g |
| Psmd11 | Hsdl2 | Dynlrb1 | Sec22b | Psmd9 | Gfer |
| Atp5e | Eif4g1 | Gjb1 | Clu | Lgmn | Hist1h1a |
| Serpina3k | Pdcd6 | Tpd52l2 | Fkbp4 | Rbpms | Rbm14 |
| Ik | Sec31a | Acp1 | Triap1 | Mrpl50 | Hspa4 |
| Fahd1 | Ube2v1 | Farsa | Cct4 | S100a1 | Ensa |
| Cfl1 | Scly | Anxa2 | Acss3 | Lsm2 | Lgals3bp |
| Dpy30 | Dnajb11 | Ndufs8 | Mbl2 | Hpcal1 | Tmed9 |
| Glyctk | Timm44 | Stip1 | Sf3b5 | Lamp2 | Atp1a1 |
| Hist1h1b | Vdac1 | Nampt | Cuta | Erp29 | Marcksl1 |
| Pdia2 | Ndufb9 | Ptbp1 | Copz1 | Myh8 | Sec24d |
| Ap2m1 | Crym | Dync1li1 | Ddrgk1 | Vps37c | Gatc |
| Txn2 | Rnase4 | Ubxn1 | Capg | Mff | Golga5 |
| Mrpl53 | Tagln | Mtss1 | Myo9b | Emcn | Clec4f |
| Rbp1 | Capns1 | Afm | Ttc32 | Nmt1 | Shank3 |
| Higd1a | Banf1 | Tgm1 | Ciapin1 | Pkp1 | Cops8 |
| Snx6 | Gimap4 | Gatm | Npepl1 | Nsd1 | Acyp2 |
| Sfpq | Hcls1 | Rab5c | Nup62 | Bcas2 | Tbc1d8b |
| Mia2 | Ak1 | Hnrnpa0 | Amotl2 | Sp1 | Npc1 |
| Rab1b | Sgta | Atp6v1e1 | Tom1 | Lmnb1 | Arsb |
| Fxn | Igbp1 | Sec24b | Lsm4 | Ltbp4 | Ssr3 |
| Tmed4 | Chmp4b | Hao2 | Slc25a12 | Ank1 | Sod3 |
| Psme3 | Cnpy3 | Orm1 | Aplp2 | Gar1 | Arhgap24 |
| Hnrnpab | Pcmt1 | Nap1l4 | Aip | Flna | Vcpip1 |
| Cmc1 | Stoml2 | Pcp4l1 | Ndufaf2 | Txlna | Mns1 |
| Pabpn1 | Hnrnpul1 | Lamp1 | Blvra | Vdac2 | Gimap8 |
| Gorasp2 | Cdc26 | Mareks | FAM120A | App | Dhrs7b |
| Smpdl3a | Mprip | Denr | Scfd1 | Cox17 | Chmp1a |
| Sf3b4 | Syap1 | Col1a2 | Csad | Myef2 | Hdac4 |
| Ehd1 | Rbp4 | Apon | Idh3b | Guk1 | Cog2 |
| G3bp2 | Pf4 | Hcfc1 | Larp1 | Scamp2 | Mtus1 |
| Enpp2 | Mustn1 | Gtf2a1 | Camp | Slc25a4 | Cab39 |
| Vwa5a | Sirt4 | Clic1 | Tnnt3 | Purb | Cnot3 |
| Ndufb2 | Diablo | Lsm8 | Kpna4 | Hccs | Gpx3 |
| Glrx3 | Tcea1 | Limd1 | Zfand2b | Cd300lg | Sipa1l3 |
| Lrp1 | Clint1 | Lsm3 | Yap1 | Tpp2 | Bmp2k |
| Epn1 | Cct3 | Scoc | Siae | Apod | Adsl |
| Mbl1 | Tmpo | Plaa | Ythdf3 | Ttn | Ngef |
| Slc38a3 | Cdkn1b | Basp1 | Eif3a | Ttc39c | Trip11 |
| Coro1b | Xpnpep1 | Arpc4 | Dnajb2 | Bst2 | Ilkap |
| Eif2a | Fam162a | Igf1 | Ppdpf | Upf1 | Egfr |
| Dtd1 | Acbd5 | Gatad2b | Tnks1bp1 | Pxn | Tsc22d4 |
| Mrps31 | Dctn2 | Atrx | Bcl2l13 | Dnm11 | Ccdc90b |
| Grb2 | Psmd2 | Gltp | Nenf | Znrf2 | Col4a2 |
| Maged1 | Angptl3 | Armc1 | Hopx | Myh13 | Son |
| Snx5 | Vcl | Bcap31 | Arpc5l | Wipf1 | Arhgap17 |
| Fhit | Matr3 | Aldoc | Nucb2 | Man1a | Dag1 |
| Grcc10 | Eps15l1 | Apoh | Cd74 | Nrgn | Rdh13 |
| Ostc | Sec16b | Acsm3 | Evl | Dync1li2 | Ube2g1 |
| Pgd | Ywhaq | Tomm22 | Sorbs2 | Snw1 | Clec3b |
| Eif4ebp1 | Gpihbp1 | Ubqln2 | Csde1 | Naglu | Commd10 |
| Sh3bgrl3 | Sdr39u1 | Oxsr1 | Ppp3r1 | Ddx19a | Ift20 |
| Ddah2 | Sra1 | Zfand6 | Pdxdc1 | Gpld1 | Hmox1 |
| Timm50 | Slc25a11 | Lrg1 | Try10 | Epn2 | Prpf3 |
| Lsp1 | Synpo | Ccdc6 | Acbd3 | Dip2b | Git1 |
| Ddx3y | Golgb1 | Ahctf1 | Dctn3 | Cygb | Gga2 |
| Eif1b | Ptk2b | Uap1l1 | Tes | Rock1 | Rbm39 |
| Wars2 | Tjp2 | Nav2 | Gypc | Dnaja2 | Tmcc3 |
| Irf2bp2 | Appl1 | Pbx1 | Cpeb4 | Slc47a1 | Mvd |
| Cnnl1 | Hdac5 | Cyp20a1 | Stk24 | Apoa5 | Gpkow |
| Obscn | Bin2 | Plcb3 | Bcl2l1 | Ppp1r1b | Col1a1 |
| Reck | Slc12a7 | Gkap1 | Nufip2 | Nup153 | Snapin |
| Ube2c | Sart1 | Bdh2 | Vti1b | Golga4 | Nisch |
| Tmem109 | Rrs1 | Cdk2ap1 | Tor1aip1 | Lemd3 | Thap4 |
| Kdelc1 | Pde1a | Amfr | Polr1d | Atad2b | Bid |
| Ythdf2 | Shroom2 | Plekha7 | Mkl2 | Rsl1d1 | Pxdn |
| Tnnc1 | Cdkn2aip | Sh3gl2 | Cisd2 | Nck1 | Inpp5d |
| Mkrn1 | Ncoa1 | Ly6d | Dhx29 | Stat1 | Vasp |
| Il16 | Itgb1 | Tff2 | Gng2 | Lsm14a | Zc3h4 |
| Hs1bp3 | Tom1l2 | Med11 | Nup50 | Cryl1 | Arhgef12 |
| Afap1l2 | Myo5b | Trip12 | Hmbox1 | Llgl2 | Stk4 |
| Zwint | Golga2 | Rnf214 | Hdac6 | Tbc1d1 | Slc5a8 |
| Ssna1 | Mtfr1 | Pdlim3 | Postn | Ranbp1 | Myo18a |
| Dnajb6 | Atxn3 | Crebbp | Osbpl3 | Snap23 | Gigyf2 |
| Mrpl40 | Evc | Tubgcp3 | Anks4b | Baiap2 | Rilp |
| Lpl | Cgref1 | Slc4a4 | Plekhg3 | Cwc15 | Dnajc1 |
| Tbl1xr1 | Ubap1 | Ctnnbip1 | Tax1bp1 | Gtf2i | Clcc1 |
| Strn4 | Usp47 | U2af2 | Ift140 | Rab3ip | Phyhipl |
| Zyx | Thoc2 | Cdc42ep4 | Bin1 | Ctcf | Mapre1 |
| Rarres2 | Galns | Psip1 | Sncg | Snx4 | Sec16a |
| Spast | Hmgb2 | Clasp2 | Notch2 | Spats2 | Lemd2 |
| Ddn | Dus3l | Bad | Akr1c18 | Faf2 | Elavl1 |
| Nhlrc3 | Sorbs3 | Gipc2 | Sdhaf1 | Coq5 | Plcl1 |
| Tmem51 | Sin3a | Bmpr2 | Zc3h11a | Glod5 | Ranbp3 |
| Supv3l1 | Ppp1r10 | Cdh11 | Ptpn2 | Irf3 | Wasf2 |
| Tarsl2 | Smtnl2 | Dao | Agfg1 | Ap3d1 | Plekha6 |
| Cdkn2c | Cand1 | Lama5 | Ect2 | Mta2 | Nsfl1c |
| Pcif1 | Cpm | Rbm6 | Arih1 | Ccdc12 | Sntb2 |
| Igkv12-46 | Rasip1 | Ppm1h | Sh3glb1 | Ubap2 | Tbcc |
| Amdhd2 | Srp68 | Myo1c | Sltm | Amn | Cnn2 |
| Pik3r4 | Pex1 | Cnot2 | Arhgap6 | Tppp3 | Eps8 |
| Spa17 | Abi1 | Xab2 | Ube3a | Rnf2 | Pds5a |
| Soat1 | Pcf11 | Slc12a3 | Sqstm1 | Ankhd1 | Limch1 |
| Bod11 | Tdrd3 | Mrps14 | Cherp | Prnp | Hbb-y |
| R3hdm1 | Clip2 | Ppig | Gopc | Pcdh1 | Ankrd1l7 |
| Chordc1 | Snap47 | Bsdc1 | Usp8 | Tpr | Rtf1 |
| Lmtk2 | Exoc6 | Ncor1 | Ubqln4 | Tcof1 | Slc12a1 |
| Pycard | Cux1 | Dnajb4 | Atp6v1g3 | Col15a1 | Ndufa13 |
| Hars | Sf3a1 | Huwe1 | Gas2 | Mrpl43 | Stx7 |
| Trim28 | Spag9 | Atp1b1 | Mylpf | Gosr1 | Acy3 |
| Slk | Arfip2 | Ylpm1 | Cst6 | Stmn2 | Calb1 |
| Mapt | Ubap2l | Samd9l | Reps1 | Habp4 | Sumf2 |
| Bag5 | Mep1a | Cirbp | Alpl | Ndrg1 | Triobp |
| Eml3 | Nedd1 | Dnajb12 | Parp3 | Ablim1 | Susd2 |
| Otud7b | Clic4 | Gramd1b | Nudt19 | Dnajb1 | Dnajc2 |
| Afg3l2 | Srp72 | Ripk1 | Saa4 | Tpd52 | Nt5dc3 |
| Numb | Tfam | Thrap3 | Zfyve19 | Dnajc19 | Snx1 |
| Fhl1 | Pank1 | Parva | Ptk2 | Acyp1 | Myl1 |
| Rsu1 | Wbp11 | Folr1 | Pacsin2 | Ndufa7 | Chchd6 |
| Mmgt1 | Aak1 | Osbpl8 | Sf3b2 | Snrpb2 | Eny2 |
| Ccdc50 | Sdc4 | Pum1 | Espn | Sftpb | Col18a1 |
| Tacc2 | Pik3c2a | Ccdc47 | Itih5 | Dbnl | Zeb2 |
| Eef1e1 | Hddc3 | Ptma | Ssbp1 | Igfbp7 | Myo6 |
| Arhgef18 | Cfd | Zc3h14 | Gm11992 | Rbm3 | Add3 |
| Exoc3 | Acox3 | Gosr2 | Osbpl6 | Msh6 | Chchd1 |
| Atg16l1 | Aftph | Nqo1 | Stk3 | Dync1i2 | Dtnb |
| Lum | Luzp1 | Lrrfip2 | Itsn2 | Cda | Ndufb5 |
| Ubxn7 | Ccdc9 | Hnrnpc | Oxct1 | Arpc1a | Snrpa |
| Pin1 | Cc2d1b | Rab11fip3 | Strn3 | Lin7c | Hnrnpm |
| Phactr2 | Pawr | Coro1a | Hnrnph3 | Ppp1r12a | Ppp1r1a |
| Snap29 | Serpinh1 | Stx6 | Tjp1 | Pdzd11 | Mtdh |
| Ly6a | Sp3 | Brd4 | Agrn | Vamp8 | Ppp1r12c |
| Cab39l | Fkbpl5 | Kdm4a | Eif3g | Mrpl41 | Tagln2 |
| Bola2 | Atp6v1a | Atxn2 | Pak3 | Mybbp1a | Cttn |
| Sipa1l1 | Pdcd10 | Tomm34 | Palld | Mrps26 | Dnajc8 |
| Pkn1 | Nid2 | Arhgap18 | Tmod3 | Cst3 | Lmo7 |
| Tmx4 | Mylk | Rbm27 | Scamp1 | Hsp90ab1 | Akr1c21 |
| Fnbp11 | Dido1 | Bola3 | Sorbs1 | Ccdc124 | Cd2ap |
| Acin1 | Nup98 | Pip4k2c | Eml4 | Ddx11 | Snx2 |
| Clmn | Mep1b | Mrps36 | Pfdn2 | Phactr4 | Add1 |
| Mpp1 | Snx18 | Ccar1 | Tcn2 | Atp51 | Serbp1 |
| Vill | Pex14 | Slc12a2 | Ptprk | Cgnl1 | Eno3 |
| Akap8 | Ubtf | Csrp1 | Pdlim2 | Csrp2 | Ywhab |
| Ppp1r12b | Smtn | Ubac2 | Plvap | Ambp | Eps8l2 |
| Bsnd | Iqgap1 | Arfgap3 | Arhgef2 | Sf3b1 | Rab11fip5 |
| Aif1 | Hscb | Crkl | Trip10 | Rad23b | 2210011C24Rik |
| Aif1l | Ranbp2 | Kif5b | Fnbp1 | Ndufs4 | Calml4 |
| Psmb1 | Ttc9c | Tubb2b | Fam107b | Rufy3 | C4b |
| C2cd2l | Crk | Swap70 | Anpep | Numa1 | Cobll1 |
| Dpep1 | Dnajc12 | Guca2b | Ptpn11 | Lima1 | Slc9a3r2 |
| Ank3 | Palm | Sept7 | Dab2 | Ndufa8 | Vil1 |
| Retnlb | Tns1 | Gng12 | Cltb | Fis1 | Chchd3 |
| G3bp1 | Umod | Pfdn1 | Coro1c | Pdzk1 | Gm2a |
| Rbmx | Fus | Picalm | Lrp2 | Cald1 | Pdzk1ip1 |
| Cacybp | Ndufb6 | Apoa4 | Ahnak | Tsks | S100a6 |
| Enah | Ndufb10 | Sprr1a | Ggt1 | Uqcrb | Fxyd2 |
| Chchd7 | Fkbp3 | Atp6v1f | Pvalb | Hnrnpu | Ndufa6 |
| Sult1c2 | Hspg2 | Npm1 | Ldhb | Lrpap1 | Eif4b |
| Ctnnd1 | Plxnb2 | Cryab | Atp6v1g1 | Lyz2 | Fabp3 |
| Ndufb7 | Uqcrq | Lad1 | Lyrm4 | Cox6c | Slc9a3r1 |
| Apoa1 | Atpif1 | S100g | Hbb-b2 | | |

TABLE 9
Gene ontology by a process of the differently expressed proteins in the
liver and the kidney extracted with electroporation mapped with GOrilla

| | | | FDR | Enrichment |
|---|---|---|---|---|
| GO term | Description | P-value* | q-value** | (N, B, n, b)*** |
| GO:0044281 | small molecule metabolic | 2.34E−50 | 1.90E−46 | 2.72 (1731, 323, 333, 169) |
| | process | | | |
| GO:0006082 | organic acid metabolic | 6.13E−44 | 2.48E−40 | 3.40 (1731, 209, 285, 117) |
| | process | | | |
| GO:0043436 | oxoacid metabolic | 7.64E−43 | 2.06E−39 | 3.39 (1731, 206, 285, 115) |
| | process | | | |
| GO:0019752 | carboxylic acid metabolic | 1.87E−41 | 3.78E−38 | 3.36 (1731, 204, 285, 113) |
| | process | | | |
| GO:0055114 | oxidation-reduction | 1.13E−37 | 1.84E−34 | 2.37 (1731, 218, 510, 152 |
| | process | | | |
| GO:0044282 | small molecule catabolic | 8.09E−36 | 1.09E−32 | 4.49 (1731, 91, 292, 69) |
| | process | | | |
| GO:0008152 | metabolic process | 4.76E−35 | 5.51E−32 | 1.42 (1731, 914, 547, 411) |
| GO:0016054 | organic acid catabolic | 2.93E−30 | 2.97E−27 | 4.72 (1731, 72, 285, 56 |
| | process | | | |
| GO:0046395 | carboxylic acid catabolic | 2.93E−30 | 2.64E−27 | 4.72 (1731, 72, 285, 56) |
| | process | | | |
| GO:0071704 | organic substance | 6.56E−30 | 5.32E−27 | 1.46 (1731, 816, 524, 360) |
| | metabolic process | | | |
| GO:0051186 | cofactor metabolic | 6.16E−27 | 4.54E−24 | 2.60 (1731, 145, 460, 100) |
| | process | | | |
| GO:0032787 | monocarboxylic acid | 6.35E−27 | 4.29E−24 | 2.74 (1731, 116, 480, 88) |
| | metabolic process | | | |
| GO:0017144 | drug metabolic process | 3.02E−26 | 1.88E−23 | 2.60 (1731, 143, 457, 98) |
| GO:0044237 | cellular metabolic process | 5.98E−26 | 3.46E−23 | 1.44 (1731, 794, 524, 346) |
| GO:0009056 | catabolic process | 2.33E−24 | 1.26E−21 | 2.43 (1731, 279, 294, 115) |

*‘P-value’ is the enrichment p-value computed according to the mHG or HG model. This p-value is not corrected for multiple testing of 731 GO terms.
**‘FDR q-value’ is the correction of the above p-value for multiple testing using the Benjamini and Hochberg (1995) method.
Namely, for the i^{th} term (ranked according to p-value) the FDR q-value is (p-value * a number of GO terms)/i.
***Enrichment (N, B, n, b) is defined as follows:
N - is the total number of genes
B - is the total number of genes associated with a specific GO term
n - is the number of genes in the top of the user's input list or in the target set when appropriate
b - is the number of genes in the intersection
Enrichment = (b/n)/(B/N)

Analysis of the function shows multiple significant functional differences between the liver and the kidney, these including catalytic activity, drug binding, and fatty-acyl-CoA binding, lyase activity, oxidoreductase activities expressed higher in the liver consistent with literature (Table 10, Table 11).

TABLE 10
Gene ontology by a function of the differently expressed proteins in the
liver and the kidney extracted with electroporation mapped with Gorilla

| | | | FDR | Enrichment |
|---|---|---|---|---|
| GO term | Description | P-value* | q-value** | (N, B, n, b)*** |
| GO:0003824 | catalytic activity | 4.64E−50 | 9.28E−47 | 1.75 (1731, 672, 480, 326) |
| GO:0016491 | oxidoreductase activity | 1.23E−37 | 1.23E−34 | 2.78 (1731, 197, 398, 126) |
| GO:0048037 | cofactor binding | 2.47E−27 | 1.64E−24 | 2.56 (1731, 141, 484, 101) |
| GO:0050662 | coenzyme binding | 9.02E−21 | 4.5E−18 | 3.12 (1731, 93, 376, 63) |
| GO:0036094 | small molecule binding | 2.65E−14 | 1.06E−11 | 2.13 (1731, 336, 230, 95) |
| GO:0031406 | carboxylic acid binding | 5.16E−12 | 1.72E−9 | 3.58 (1731, 49, 316, 32) |
| GO:0016829 | lyase activity | 1.47E−11 | 4.21E−9 | 3.50 (1731, 46, 333, 31) |
| GO:0043177 | organic acid binding | 2.7E−11 | 6.74E−9 | 3.44 (1731, 51, 316, 32) |
| GO:0042802 | identical protein binding | 6.05E−11 | 1.34E−8 | 2.46 (1731, 299, 127, 54) |
| GO:1901265 | nucleoside phosphate | 2.98E−9 | 5.95E−7 | 1.43 (1731, 257, 743, 158) |
| | binding | | | |
| GO:0000166 | nucleotide binding | 2.98E−9 | 5.41E−7 | 1.43 (1731, 257, 743, 158) |
| GO:0016616 | oxidoreductase activity, | 4.38E−9 | 7.3E−7 | 4.04 (1731, 43, 229, 23) |
| | acting on the CH—OH | | | |
| | group of donors, NAD or | | | |
| | NADP as acceptor | | | |
| GO:0016836 | hydro-lyase activity | 5.24E−9 | 8.05E−7 | 6.79 (1731, 17, 195, 13) |
| GO:0051287 | NAD binding | 6.27E−9 | 8.94E−7 | 3.31 (1731, 29, 415, 23) |
| GO:0003735 | structural constituent of | 1.69E−8 | 2.25E−6 | 2.67 (1731, 35, 518, 28) |
| | ribosome | | | |

*‘P-value’ is the enrichment p-value computed according to the mHG or HG model. This p-value is not corrected for multiple testing of 731 GO terms.
**‘FDR q-value’ is the correction of the above p-value for multiple testing using the Benjamini and Hochberg (1995) method.
Namely, for the i^{th} term (ranked according to p-value) the FDR q-value is (p-value * a number of GO terms)/i.
***Enrichment (N, B, n, b) is defined as follows:
N - is the total number of genes
B - is the total number of genes associated with a specific GO term
n - is the number of genes in the top of the user's input list or in the target set when appropriate
b - is the number of genes in the intersection
Enrichment = (b/n)/(B/N)

TABLE 11

| | | | FDR q- | | | | | |
|---|---|---|---|---|---|---|---|---|
| GO Term | Description | P-value | value | Enrichment | N | B | n | b |
| GO:0003824 | catalytic activity | 4.64E−50 | 9.28E−47 | 1.75 | 1731 | 672 | 480 | 326 |
| GO:0016491 | oxidoreductase activity | 1.23E−37 | 1.23E−34 | 2.78 | 1731 | 197 | 398 | 126 |
| GO:0048037 | cofactor binding | 2.47E−27 | 1.64E−24 | 2.56 | 1731 | 141 | 484 | 101 |
| GO:0050662 | coenzyme binding | 9.02E−21 | 4.50E−18 | 3.12 | 1731 | 93 | 376 | 63 |
| GO:0036094 | small molecule binding | 2.65E−14 | 1.06E−11 | 2.13 | 1731 | 336 | 230 | 95 |
| GO:0031406 | carboxylic acid binding | 5.16E−12 | 1.72E−09 | 3.58 | 1731 | 49 | 316 | 32 |
| GO:0016829 | lyase activity | 1.47E−11 | 4.21E−09 | 3.5 | 1731 | 46 | 333 | 31 |
| GO:0043177 | organic acid binding | 2.70E−11 | 6.74E−09 | 3.44 | 1731 | 51 | 316 | 32 |
| GO:0042802 | identical protein binding | 6.05E−11 | 1.34E−08 | 2.46 | 1731 | 299 | 127 | 54 |
| GO:1901265 | nucleoside phosphate | 2.98E−09 | 5.95E−07 | 1.43 | 1731 | 257 | 743 | 158 |
| | binding | | | | | | | |
| GO:0000166 | nucleotide binding | 2.98E−09 | 5.41E−07 | 1.43 | 1731 | 257 | 743 | 158 |
| GO:0016616 | oxidoreductase activity, | 4.38E−09 | 7.30E−07 | 4.04 | 1731 | 43 | 229 | 23 |
| | acting on the CH—OH | | | | | | | |
| | group of donors, NAD | | | | | | | |
| | or NADP as acceptor | | | | | | | |
| GO:0016836 | hydro-lyase activity | 5.24E−09 | 8.05E−07 | 6.79 | 1731 | 17 | 195 | 13 |
| GO:0051287 | NAD binding | 6.27E−09 | 8.94E−07 | 3.31 | 1731 | 29 | 415 | 23 |
| GO:0003735 | structural constituent of | 1.69E−08 | 2.25E−06 | 2.67 | 1731 | 35 | 518 | 28 |
| | ribosome | | | | | | | |
| GO:0016614 | oxidoreductase activity, | 1.87E−08 | 2.34E−06 | 2.62 | 1731 | 46 | 460 | 32 |
| | acting on CH—OH group | | | | | | | |
| | of donors | | | | | | | |
| GO:0016620 | oxidoreductase activity, | 4.74E−08 | 5.57E−06 | 3.6 | 1731 | 19 | 430 | 17 |
| | acting on the aldehyde | | | | | | | |
| | or oxo group of donors, | | | | | | | |
| | NAD or NADP as | | | | | | | |
| | acceptor | | | | | | | |
| GO:0016853 | isomerase activity | 5.26E−08 | 5.84E−06 | 2.31 | 1731 | 37 | 628 | 31 |
| GO:0016835 | carbon-oxygen lyase | 5.38E−08 | 5.66E−06 | 6.07 | 1731 | 19 | 195 | 13 |
| | activity | | | | | | | |
| GO:0043167 | ion binding | 6.73E−08 | 6.72E−06 | 1.93 | 1731 | 614 | 76 | 52 |
| GO:0016903 | oxidoreductase activity, | 7.58E−08 | 7.21E−06 | 3.45 | 1731 | 21 | 430 | 18 |
| | acting on the aldehyde | | | | | | | |
| | or oxo group of donors | | | | | | | |
| GO:0016765 | transferase activity, | 2.39E−07 | 2.17E−05 | 4.16 | 1731 | 12 | 416 | 12 |
| | transferring alkyl or aryl | | | | | | | |
| | (other than methyl) | | | | | | | |
| | groups | | | | | | | |
| GO:0050660 | flavin adenine | 2.49E−07 | 2.16E−05 | 3.01 | 1731 | 24 | 480 | 20 |
| | dinucleotide binding | | | | | | | |
| GO:0043168 | anion binding | 2.63E−07 | 2.19E−05 | 1.77 | 1731 | 337 | 221 | 76 |
| GO:0008144 | drug binding | 3.66E−07 | 2.93E−05 | 3.23 | 1731 | 191 | 73 | 26 |
| GO:0016860 | intramolecular | 6.25E−07 | 4.80E−05 | 3.5 | 1731 | 13 | 495 | 13 |
| | oxidoreductase activity | | | | | | | |
| GO:0016597 | amino acid binding | 8.89E−07 | 6.58E−05 | 12.15 | 1731 | 19 | 60 | 8 |
| GO:0016209 | antioxidant activity | 1.07E−06 | 7.63E−05 | 6.44 | 1731 | 31 | 104 | 12 |
| GO:0003988 | acetyl-CoA C- | 1.25E−06 | 8.62E−05 | 11.62 | 1731 | 6 | 149 | 6 |
| | acyltransferase activity | | | | | | | |
| GO:0016645 | oxidoreductase activity, | 1.72E−06 | 1.15E−04 | 6.06 | 1731 | 10 | 257 | 9 |
| | acting on the CH—NH | | | | | | | |
| | group of donors | | | | | | | |
| GO:0004029 | aldehyde dehydrogenase | 1.90E−06 | 1.22E−04 | 9.47 | 1731 | 8 | 160 | 7 |
| | (NAD) activity | | | | | | | |
| GO:1901567 | fatty acid derivative | 2.43E−06 | 1.52E−04 | 5.46 | 1731 | 16 | 218 | 11 |
| | binding | | | | | | | |
| GO:0033218 | amide binding | 3.29E−06 | 1.99E−04 | 2.9 | 1731 | 61 | 245 | 25 |
| GO:0050661 | NADP binding | 5.66E−06 | 3.33E−04 | 3.74 | 1731 | 16 | 376 | 13 |
| GO:0019842 | vitamin binding | 6.76E−06 | 3.86E−04 | 3.83 | 1731 | 33 | 219 | 16 |
| GO:0009055 | electron transfer activity | 7.06E−06 | 3.92E−04 | 2.55 | 1731 | 30 | 498 | 22 |
| GO:0042803 | protein | 1.02E−05 | 5.48E−04 | 2.26 | 1731 | 143 | 198 | 37 |
| | homodimerization | | | | | | | |
| | activity | | | | | | | |
| GO:0016627 | oxidoreductase activity, | 1.05E−05 | 5.55E−04 | 4.04 | 1731 | 25 | 240 | 14 |
| | acting on the CH—CH | | | | | | | |
| | group of donors | | | | | | | |
| GO:0097159 | organic cyclic | 1.19E−05 | 6.11E−04 | 1.38 | 1731 | 556 | 307 | 136 |
| | compound binding | | | | | | | |
| GO:0016787 | hydrolase activity | 1.37E−05 | 6.86E−04 | 1.32 | 1731 | 273 | 720 | 150 |
| GO:0004364 | glutathione transferase | 1.48E−05 | 7.20E−04 | 4.16 | 1731 | 9 | 416 | 9 |
| | activity | | | | | | | |
| GO:0016740 | transferase activity | 2.40E−05 | 1.14E−03 | 1.88 | 1731 | 165 | 285 | 51 |
| GO:0000062 | fatty-acyl-CoA binding | 2.53E−05 | 1.18E−03 | 5.5 | 1731 | 13 | 218 | 9 |
| GO:0005506 | iron ion binding | 3.49E−05 | 1.58E−03 | 3.03 | 1731 | 20 | 428 | 15 |
| GO:1901363 | heterocyclic compound | 4.34E−05 | 1.93E−03 | 1.37 | 1731 | 536 | 307 | 130 |
| | binding | | | | | | | |
| GO:0016408 | C-acyltransferase | 4.55E−05 | 1.98E−03 | 8.71 | 1731 | 8 | 149 | 6 |
| | activity | | | | | | | |
| GO:0072341 | modified amino acid | 4.89E−05 | 2.08E−03 | 5.8 | 1731 | 17 | 158 | 9 |
| | binding | | | | | | | |
| GO:0003857 | 3-hydroxyacyl-CoA | 5.38E−05 | 2.24E−03 | 7.61 | 1731 | 7 | 195 | 6 |
| | dehydrogenase activity | | | | | | | |
| GO:0016684 | oxidoreductase activity, | 6.70E−05 | 2.73E−03 | 7.01 | 1731 | 19 | 104 | 8 |
| | acting on peroxide as | | | | | | | |
| | acceptor | | | | | | | |
| GO:0015036 | disulfide oxidoreductase | 6.74E−05 | 2.69E−03 | 2.19 | 1731 | 15 | 789 | 15 |
| | activity | | | | | | | |
| GO:0046983 | protein dimerization | 7.45E−05 | 2.92E−03 | 1.98 | 1731 | 182 | 202 | 42 |
| | activity | | | | | | | |
| GO:0016705 | oxidoreductase activity, | 7.78E−05 | 2.99E−03 | 2.68 | 1731 | 24 | 457 | 17 |
| | acting on paired donors, | | | | | | | |
| | with incorporation or | | | | | | | |
| | reduction of molecular | | | | | | | |
| | oxygen | | | | | | | |
| GO:0016667 | oxidoreductase activity, | 8.24E−05 | 3.11E−03 | 2.08 | 1731 | 19 | 789 | 18 |
| | acting on a sulfur group | | | | | | | |
| | of donors | | | | | | | |
| GO:0008483 | transaminase activity | 9.59E−05 | 3.55E−03 | 8.05 | 1731 | 5 | 215 | 5 |
| GO:0033293 | monocarboxylic acid | 1.04E−04 | 3.77E−03 | 3.28 | 1731 | 19 | 361 | 13 |
| | binding | | | | | | | |
| GO:0016830 | carbon-carbon lyase | 1.05E−04 | 3.74E−03 | 3.57 | 1731 | 18 | 323 | 12 |
| | activity | | | | | | | |
| GO:0043169 | cation binding | 1.13E−04 | 3.97E−03 | 2.2 | 1731 | 368 | 60 | 28 |
| GO:0003995 | acyl-CoA | 1.16E−04 | 3.99E−03 | 6.81 | 1731 | 7 | 218 | 6 |
| | dehydrogenase activity | | | | | | | |
| GO:0005504 | fatty acid binding | 1.27E−04 | 4.31E−03 | 2.72 | 1731 | 17 | 524 | 14 |
| GO:0008395 | steroid hydroxylase | 1.30E−04 | 4.34E−03 | 3.79 | 1731 | 8 | 457 | 8 |
| | activity | | | | | | | |
| GO:0046914 | transition metal ion | 1.36E−04 | 4.46E−03 | 2.58 | 1731 | 122 | 132 | 24 |
| | binding | | | | | | | |
| GO:0000287 | magnesium ion binding | 1.47E−04 | 4.72E−03 | 2.26 | 1731 | 30 | 535 | 21 |
| GO:0051213 | dioxygenase activity | 1.64E−04 | 5.20E−03 | 17.35 | 1731 | 7 | 57 | 4 |
| GO:0003674 | molecular function | 2.05E−04 | 6.40E−03 | 1.02 | 1731 | 1672 | 959 | 942 |
| GO:0016453 | C-acetyltransferase | 2.12E−04 | 6.50E−03 | 20.61 | 1731 | 3 | 84 | 3 |
| | activity | | | | | | | |
| GO:0003985 | acetyl-CoA C- | 2.12E−04 | 6.40E−03 | 20.61 | 1731 | 3 | 84 | 3 |
| | acetyltransferase activity | | | | | | | |
| GO:0004497 | monooxygenase activity | 2.24E−04 | 6.68E−03 | 2.79 | 1731 | 19 | 457 | 14 |
| GO:0016874 | ligase activity | 2.40E−04 | 7.04E−03 | 1.99 | 1731 | 27 | 710 | 22 |
| GO:0052689 | carboxylic ester | 2.91E−04 | 8.43E−03 | 1.76 | 1731 | 22 | 937 | 21 |
| | hydrolase activity | | | | | | | |
| GO:0016702 | oxidoreductase activity, | 3.58E−04 | 1.02E−02 | 22.78 | 1731 | 4 | 57 | 3 |
| | acting on single donors | | | | | | | |
| | with incorporation of | | | | | | | |
| | molecular oxygen, | | | | | | | |
| | incorporation of two | | | | | | | |
| | atoms of oxygen | | | | | | | |
| GO:0016701 | oxidoreductase activity, | 3.58E−04 | 1.01E−02 | 22.78 | 1731 | 4 | 57 | 3 |
| | acting on single donors | | | | | | | |
| | with incorporation of | | | | | | | |
| | molecular oxygen | | | | | | | |
| GO:0004300 | enoyl-CoA hydratase | 3.88E−04 | 1.08E−02 | 7.4 | 1731 | 6 | 195 | 5 |
| | activity | | | | | | | |
| GO:0015078 | proton transmembrane | 4.09E−04 | 1.12E−02 | 4.29 | 1731 | 24 | 168 | 10 |
| | transporter activity | | | | | | | |
| GO:0020037 | heme binding | 4.37E−04 | 1.18E−02 | 2.75 | 1731 | 25 | 378 | 15 |
| GO:0004601 | peroxidase activity | 4.44E−04 | 1.18E−02 | 6.47 | 1731 | 18 | 104 | 7 |
| GO:0004602 | glutathione peroxidase | 4.46E−04 | 1.17E−02 | 5.45 | 1731 | 7 | 272 | 6 |
| | activity | | | | | | | |
| GO:0048029 | monosaccharide binding | 4.55E−04 | 1.18E−02 | 2.1 | 1731 | 16 | 772 | 15 |
| GO:0030170 | pyridoxal phosphate | 5.10E−04 | 1.31E−02 | 3 | 1731 | 14 | 454 | 11 |
| | binding | | | | | | | |
| GO:0017076 | purine nucleotide | 5.14E−04 | 1.30E−02 | 1.33 | 1731 | 193 | 743 | 110 |
| | binding | | | | | | | |
| GO:0046872 | metal ion binding | 5.45E−04 | 1.36E−02 | 2.77 | 1731 | 360 | 26 | 15 |
| GO:0043531 | ADP binding | 5.93E−04 | 1.46E−02 | 7.9 | 1731 | 18 | 73 | 6 |
| GO:0016651 | oxidoreductase activity, | 6.07E−04 | 1.48E−02 | 2.05 | 1731 | 29 | 612 | 21 |
| | acting on NAD(P)H | | | | | | | |
| GO:0016746 | transferase activity, | 6.35E−04 | 1.53E−02 | 5.55 | 1731 | 29 | 86 | 8 |
| | transferring acyl groups | | | | | | | |
| GO:0016769 | transferase activity, | 6.70E−04 | 1.59E−02 | 6.71 | 1731 | 6 | 215 | 5 |
| | transferring nitrogenous | | | | | | | |
| | groups | | | | | | | |
| GO:0030554 | adenyl nucleotide | 6.81E−04 | 1.60E−02 | 1.86 | 1731 | 164 | 221 | 39 |
| | binding | | | | | | | |
| GO:0051920 | peroxiredoxin activity | 7.22E−04 | 1.68E−02 | 11.1 | 1731 | 6 | 104 | 4 |
| GO:0046906 | tetrapyrrole binding | 8.23E−04 | 1.89E−02 | 2.64 | 1731 | 26 | 378 | 15 |
| GO:0015643 | toxic substance binding | 8.61E−04 | 1.95E−02 | 66.58 | 1731 | 4 | 13 | 2 |
| GO:0005542 | folic acid binding | 9.24E−04 | 2.07E−02 | 9.17 | 1731 | 5 | 151 | 4 |

Analysis by component showed large differences in mitochondrion related proteins extracted from the liver vs. kidney (Table 12, Table 13).

TABLE 12
Gene ontology by a component of the differently expressed proteins in the
liver and the kidney extracted with electroporation mapped with Gorilla

| | | | FDR | Enrichment |
|---|---|---|---|---|
| GO term | Description | P-value* | q-value** | (N, B, n, b)*** |
| GO:0005739 | mitochondrion | 4.63E−37 | 5.23E−34 | 2.00 (1731, 429, 432, 214 |
| GO:0044429 | mitochondrial part | 9.3E−20 | 5.26E−17 | 1.90 (1731, 235, 542, 140) |
| GO:0044444 | cytoplasmic part | 4.7E−16 | 1.77E−13 | 1.24 (1731, 1195, 428, 366) |
| GO:0005743 | mitochondrial inner | 4.81E−16 | 1.36E−13 | 1.96 (1731, 144, 612, 100) |
| | membrane | | | |
| GO:0031966 | mitochondrial | 9.05E−16 | 2.05E−13 | 2.22 (1731, 175, 401, 90) |
| | membrane | | | |
| GO:0043209 | myelin sheath | 7.47E−15 | 1.41E−12 | 2.30 (1731, 78, 597, 62) |
| GO:0019866 | organelle inner | 2.18E−14 | 3.52E−12 | 1.97 (1731, 149, 559, 95) |
| | membrane | | | |
| GO:0043233 | organelle lumen | 1.2E−9 | 1.69E−7 | 2.30 (1731, 94, 408, 51) |
| GO:0070013 | intracellular organelle | 1.2E−9 | 1.5E−7 | 2.30 (1731, 94, 408, 51) |
| | lumen | | | |
| GO:0031974 | membrane-enclosed | 1.2E−9 | 1.35E−7 | 2.30 (1731, 94, 408, 51) |
| | lumen | | | |
| GO:0022627 | cytosolic small | 1.84E−9 | 1.89E−7 | 4.06 (1731, 20, 384, 18) |
| | ribosomal subunit | | | |
| GO:0042579 | microbody | 3.44E−9 | 3.24E−7 | 2.61 (1731, 47, 480, 34) |
| GO:0005777 | peroxisome | 9.61E−9 | 8.36E−7 | 2.59 (1731, 46, 480, 33) |
| GO:0005759 | mitochondrial matrix | 1.65E−8 | 1.33E−6 | 2.20 (1731, 48, 623, 38) |
| GO:0031090 | organelle membrane | 3.01E−7 | 2.27E−5 | 1.56 (1731, 296, 401, 107) |

*‘P-value’ is the enrichment p-value computed according to the mHG or HG model. This p-value is not corrected for multiple testing of 731 GO terms.
**‘FDR q-value’ is the correction of the above p-value for multiple testing using the Benjamini and Hochberg (1995) method.
Namely, for the i^{th} term (ranked according to p-value) the FDR q-value is (p-value * a number of GO terms)/i.
***Enrichment (N, B, n, b) is defined as follows:
N - is the total number of genes
B - is the total number of genes associated with a specific GO term
n - is the number of genes in the top of the user's input list or in the target set when appropriate
b - is the number ot genes in the intersection
Enrichment = (b/n)/(B/N)

TABLE 13

| | | | FDR q- | | | | | |
|---|---|---|---|---|---|---|---|---|
| GO Term | Description | P-value | value | Enrichment | N | B | n | b |
| GO:0005739 | mitochondrion | 4.63E−37 | 5.23E−34 | 2 | 1731 | 429 | 432 | 214 |
| GO:0044429 | mitochondrial part | 9.30E−20 | 5.26E−17 | 1.9 | 1731 | 235 | 542 | 140 |
| GO:0044444 | cytoplasmic part | 4.70E−16 | 1.77E−13 | 1.24 | 1731 | 1195 | 428 | 366 |
| GO:0005743 | mitochondrial inner | 4.81E−16 | 1.36E−13 | 1.96 | 1731 | 144 | 612 | 100 |
| | membrane | | | | | | | |
| GO:0031966 | mitochondrial | 9.05E−16 | 2.05E−13 | 2.22 | 1731 | 175 | 401 | 90 |
| | membrane | | | | | | | |
| GO:0043209 | myelin sheath | 7.47E−15 | 1.41E−12 | 2.3 | 1731 | 78 | 597 | 62 |
| GO:0019866 | organelle inner | 2.18E−14 | 3.52E−12 | 1.97 | 1731 | 149 | 559 | 95 |
| | membrane | | | | | | | |
| GO:0043233 | organelle lumen | 1.20E−09 | 1.69E−07 | 2.3 | 1731 | 94 | 408 | 51 |
| GO:0070013 | intracellular | 1.20E−09 | 1.50E−07 | 2.3 | 1731 | 94 | 408 | 51 |
| | organelle lumen | | | | | | | |
| GO:0031974 | membrane-enclosed | 1.20E−09 | 1.35E−07 | 2.3 | 1731 | 94 | 408 | 51 |
| | lumen | | | | | | | |
| GO:0022627 | cytosolic small | 1.84E−09 | 1.89E−07 | 4.06 | 1731 | 20 | 384 | 18 |
| | ribosomal subunit | | | | | | | |
| GO:0042579 | microbody | 3.44E−09 | 3.24E−07 | 2.61 | 1731 | 47 | 480 | 34 |
| GO:0005777 | peroxisome | 9.61E−09 | 8.36E−07 | 2.59 | 1731 | 46 | 480 | 33 |
| GO:0005759 | mitochondrial matrix | 1.65E−08 | 1.33E−06 | 2.2 | 1731 | 48 | 623 | 38 |
| GO:0031090 | organelle membrane | 3.01E−07 | 2.27E−05 | 1.56 | 1731 | 296 | 401 | 107 |
| GO:0043231 | intracellular | 9.01E−07 | 6.37E−05 | 1.16 | 1731 | 1166 | 409 | 320 |
| | membrane-bounded | | | | | | | |
| | organelle | | | | | | | |
| GO:0044391 | ribosomal subunit | 1.08E−06 | 7.19E−05 | 2.3 | 1731 | 45 | 518 | 31 |
| GO:0005829 | cytosol | 1.10E−06 | 6.89E−05 | 2.07 | 1731 | 504 | 68 | 41 |
| GO:0015935 | small ribosomal | 1.80E−06 | 1.07E−04 | 3.25 | 1731 | 25 | 384 | 18 |
| | subunit | | | | | | | |
| GO:0005783 | endoplasmic | 5.70E−06 | 3.22E−04 | 1.42 | 1731 | 200 | 701 | 115 |
| | reticulum | | | | | | | |
| GO:0005840 | ribosome | 6.21E−06 | 3.34E−04 | 2.53 | 1731 | 45 | 396 | 26 |
| GO:0098798 | mitochondrial protein | 1.72E−05 | 8.81E−04 | 1.52 | 1731 | 97 | 796 | 68 |
| | complex | | | | | | | |
| GO:0043227 | membrane-bounded | 4.11E−05 | 2.02E−03 | 1.13 | 1731 | 1227 | 409 | 327 |
| | organelle | | | | | | | |
| GO:0044455 | mitochondrial | 4.22E−05 | 1.99E−03 | 1.67 | 1731 | 83 | 651 | 52 |
| | membrane part | | | | | | | |
| GO:1990204 | oxidoreductase | 4.78E−05 | 2.16E−03 | 1.89 | 1731 | 52 | 618 | 35 |
| | complex | | | | | | | |
| GO:0098800 | inner mitochondrial | 7.06E−05 | 3.07E−03 | 1.73 | 1731 | 66 | 651 | 43 |
| | membrane protein | | | | | | | |
| | complex | | | | | | | |
| GO:0005782 | peroxisomal matrix | 7.32E−05 | 3.06E−03 | 247.29 | 1731 | 7 | 2 | 2 |
| GO:0031907 | microbody lumen | 7.32E−05 | 2.96E−03 | 247.29 | 1731 | 7 | 2 | 2 |
| GO:0005751 | mitochondrial | 1.54E−04 | 5.98E−03 | 7.46 | 1731 | 5 | 232 | 5 |
| | respiratory chain | | | | | | | |
| | complex IV | | | | | | | |
| GO:0005758 | mitochondrial | 1.54E−04 | 5.82E−03 | 2.68 | 1731 | 27 | 406 | 17 |
| | intermembrane space | | | | | | | |
| GO:0044424 | intracellular part | 2.16E−04 | 7.88E−03 | 1.04 | 1731 | 1533 | 841 | 773 |
| GO:0042645 | mitochondrial | 2.59E−04 | 9.13E−03 | 3.42 | 1731 | 20 | 304 | 12 |
| | nucleoid | | | | | | | |
| GO:0009295 | nucleoid | 2.59E−04 | 8.85E−03 | 3.42 | 1731 | 20 | 304 | 12 |
| GO:0022625 | cytosolic large | 3.39E−04 | 1.13E−02 | 3.04 | 1731 | 11 | 518 | 10 |
| | ribosomal subunit | | | | | | | |
| GO:0045259 | proton-transporting | 5.00E−04 | 1.61E−02 | 2.67 | 1731 | 12 | 595 | 11 |
| | ATP synthase | | | | | | | |
| | complex | | | | | | | |
| GO:0005753 | mitochondrial | 5.00E−04 | 1.57E−02 | 2.67 | 1731 | 12 | 595 | 11 |
| | proton-transporting | | | | | | | |
| | ATP synthase | | | | | | | |
| | complex | | | | | | | |
| GO:0005615 | extracellular space | 5.10E−04 | 1.56E−02 | 1.25 | 1731 | 190 | 980 | 134 |
| GO:0045277 | respiratory chain | 5.42E−04 | 1.61E−02 | 3.66 | 1731 | 7 | 473 | 7 |
| | complex IV | | | | | | | |
| GO:0044438 | microbody part | 7.66E−04 | 2.22E−02 | 2.81 | 1731 | 20 | 401 | 13 |
| GO:0044439 | peroxisomal part | 7.66E−04 | 2.16E−02 | 2.81 | 1731 | 20 | 401 | 13 |
| GO:0042788 | polysomal ribosome | 9.97E−04 | 2.75E−02 | 3.36 | 1731 | 9 | 458 | 8 |

RNA and Proteins Differential Expression with e-Biopsy in HepG2 Human Tumor Model and Normal Liver in the Mouse.

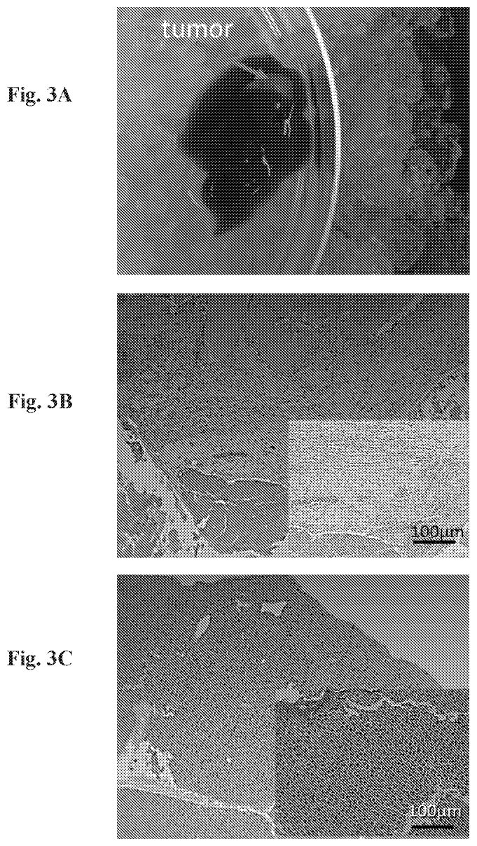

The example of a HepG2 tumor in a mice liver is shown in FIG. 3A. Histological examination clearly shows abnormal cells and tissue structures at the tumor area (FIG. 3B) vs. a normal liver structure (FIG. 3C).

It was found that in the extracts from the HepG2 liver model in mice, RNA encoding for PLK_1, S100P, TMED3, TMSB10, and KIF23 were significantly higher expressed than RNA for these genes extracted from normal liver (FIG. 4).

As in the previous section, using semi-quantitative proteomic data, the following parameters were calculated for proteins extracted from the HepG2 tumor (Table 13): molecular weight (MW), normalized intensity for each sample (LFQ), and intensity and normalized within sample intensity (iBAQ). Using these quantitative data, we selected the list of most abundant proteins with iBAQ>10⁷ for further analysis (Table 3). Histogram and density functions suggested that proteins extracted by e-biopsy have a heavy right tail distribution function. The skewness and kurtosis plots of MW suggested that MW has lognormal, gamma or Weibull distributions. The goodness of fit analysis (Table 14) suggests that MW of the most abundant proteins extracted by PEF is closer to lognormal distribution (smallest statistics for all checked criteria) (Table 15).

TABLE 14
The goodness of fit analysis of highly abundant electroporation
extracted HepG2 proteins (iBAQ > 10⁷)

| | | |
|---|---|---|
| Weibull | lognormal | gamma |

Goodness-of-fit statistics

| | | | |
|---|---|---|---|
| Kolmogorov-Smirnov statistic | 0.08502379 | 0.02567092 | 0.05181307 |
| Cramer-von Mises statistic | 2.15728759 | 0.16256876 | 1.04995599 |
| Anderson-Darling statistic | 17.59556665 | 1.31084115 | 7.67302556 |

Goodness-of-fit criteria

| | | | |
|---|---|---|---|
| Akaike's Information Criterion | 11712.26 | 11440.62 | 11584.03 |
| Bayesian Information Criterion | 11722.56 | 11450.91 | 11594.33 |

TABLE 15
Parametric bootstrap medians and 95% percentile CI for lognormal
distribution of the MW of PEF extracted HepG2 proteins

| | Median | 2.5% | 97.5% |
|---|---|---|---|
| meanlog | 3.4474497 | 3.4095316 | 3.486965 |
| sdlog | 0.6928734 | 0.6671016 | 0.719039 |

2782 proteins from HepG2 and normal liver were identified using unlabeled proteomic. Gene ontology analysis was performed for the associated genes (on the ranked list of differently expressed proteins, Table 1) using GOrilla, annotating the ranked gene list to the mouse genome. Analysis of the gene anthology by processes showed that macromolecules metabolic processes, nucleic acid metabolic processes, regulation of cellular processes and macromolecule biosynthesis processes were higher in HepG2 than in normal liver (Table 12, and Table 16).

TABLE 16

| GO Term | Description | P-value | FDR q-value | Enrichment | N | B | n | b |
|---|---|---|---|---|---|---|---|---|
| GO:0043170 | macromolecule metabolic | 2.47E−23 | 2.22E−19 | 1.36 | 2589 | 919 | 992 | 478 |
| | process | | | | | | | |
| GO:0044260 | cellular macromolecule | 1.98E−21 | 8.93E−18 | 1.44 | 2589 | 648 | 992 | 358 |
| | metabolic process | | | | | | | |
| GO:0090304 | nucleic acid metabolic | 5.93E−20 | 1.78E−16 | 1.64 | 2589 | 334 | 992 | 210 |
| | process | | | | | | | |
| GO:0050789 | regulation of biological | 7.73E−20 | 1.74E−16 | 1.25 | 2589 | 1301 | 968 | 607 |
| | process | | | | | | | |
| GO:0051171 | regulation of nitrogen | 1.08E−19 | 1.94E−16 | 1.57 | 2589 | 695 | 639 | 269 |
| | compound metabolic | | | | | | | |
| | process | | | | | | | |
| GO:0050794 | regulation of cellular | 1.55E−19 | 2.33E−16 | 1.27 | 2589 | 1202 | 970 | 570 |
| | process | | | | | | | |
| GO:0060255 | regulation of | 1.86E−19 | 2.40E−16 | 1.52 | 2589 | 718 | 689 | 291 |
| | macromolecule metabolic | | | | | | | |
| | process | | | | | | | |
| GO:0080090 | regulation of primary | 8.83E−19 | 9.95E−16 | 1.54 | 2589 | 731 | 639 | 277 |
| | metabolic process | | | | | | | |
| GO:0006412 | translation | 1.07E−16 | 1.07E−13 | 2.83 | 2589 | 161 | 398 | 70 |
| GO:0048519 | negative regulation of | 1.57E−16 | 1.41E−13 | 1.45 | 2589 | 720 | 759 | 306 |
| | biological process | | | | | | | |
| GO:0034645 | cellular macromolecule | 2.77E−16 | 2.27E−13 | 2.5 | 2589 | 231 | 381 | 85 |
| | biosynthetic process | | | | | | | |
| GO:0009059 | macromolecule | 4.38E−16 | 3.29E−13 | 2.45 | 2589 | 241 | 381 | 87 |
| | biosynthetic process | | | | | | | |
| GO:0048523 | negative regulation of | 4.78E−16 | 3.31E−13 | 1.47 | 2589 | 655 | 759 | 283 |
| | cellular process | | | | | | | |
| GO:0043043 | peptide biosynthetic | 5.81E−16 | 3.74E−13 | 2.76 | 2589 | 165 | 398 | 70 |
| | process | | | | | | | |
| GO:0010468 | regulation of gene | 7.68E−16 | 4.61E−13 | 1.53 | 2589 | 480 | 846 | 240 |
| | expression | | | | | | | |
| GO:0031323 | regulation of cellular | 9.09E−16 | 5.12E−13 | 1.52 | 2589 | 751 | 574 | 253 |
| | metabolic process | | | | | | | |
| GO:0031326 | regulation of cellular | 5.40E−15 | 2.86E−12 | 1.55 | 2589 | 441 | 838 | 221 |
| | biosynthetic process | | | | | | | |
| GO:0019222 | regulation of metabolic | 6.45E−15 | 3.23E−12 | 1.41 | 2589 | 829 | 689 | 312 |
| | process | | | | | | | |
| GO:0016070 | RNA metabolic process | 1.91E−14 | 9.06E−12 | 1.63 | 2589 | 263 | 992 | 164 |
| GO:0048518 | positive regulation of | 2.76E−14 | 1.24E−11 | 1.41 | 2589 | 814 | 701 | 310 |
| | biological process | | | | | | | |
| GO:0010556 | regulation of | 2.82E−14 | 1.21E−11 | 1.56 | 2589 | 405 | 838 | 205 |
| | macromolecule | | | | | | | |
| | biosynthetic process | | | | | | | |
| GO:0044267 | cellular protein metabolic | 2.95E−14 | 1.21E−11 | 1.52 | 2589 | 450 | 820 | 216 |
| | process | | | | | | | |
| GO:0032268 | regulation of cellular | 5.97E−14 | 2.34E−11 | 1.8 | 2589 | 394 | 497 | 136 |
| | protein metabolic process | | | | | | | |
| GO:0051246 | regulation of protein | 6.00E−14 | 2.25E−11 | 1.77 | 2589 | 430 | 513 | 151 |
| | metabolic process | | | | | | | |
| GO:0065007 | biological regulation | 6.15E−14 | 2.22E−11 | 1.23 | 2589 | 1417 | 759 | 511 |
| GO:2000112 | regulation of cellular | 7.39E−14 | 2.56E−11 | 1.57 | 2589 | 390 | 838 | 198 |
| | macromolecule | | | | | | | |
| | biosynthetic process | | | | | | | |
| GO:0010604 | positive regulation of | 7.91E−14 | 2.64E−11 | 1.67 | 2589 | 420 | 634 | 172 |
| | macromolecule metabolic | | | | | | | |
| | process | | | | | | | |
| GO:0019538 | protein metabolic process | 1.05E−13 | 3.39E−11 | 1.4 | 2589 | 649 | 822 | 288 |
| GO:0009889 | regulation of biosynthetic | 1.51E−13 | 4.68E−11 | 1.5 | 2589 | 466 | 838 | 227 |
| | process | | | | | | | |
| GO:0019219 | regulation of nucleobase- | 1.92E−13 | 5.78E−11 | 1.48 | 2589 | 402 | 989 | 227 |
| | containing compound | | | | | | | |
| | metabolic process | | | | | | | |
| GO:0048522 | positive regulation of | 5.71E−13 | 1.66E−10 | 1.43 | 2589 | 714 | 701 | 276 |
| | cellular process | | | | | | | |
| GO:0010605 | negative regulation of | 6.82E−13 | 1.92E−10 | 1.66 | 2589 | 354 | 675 | 153 |
| | macromolecule metabolic | | | | | | | |
| | process | | | | | | | |
| GO:0043604 | amide biosynthetic process | 7.19E−13 | 1.96E−10 | 2.38 | 2589 | 197 | 398 | 72 |
| GO:0006518 | peptide metabolic process | 7.81E−13 | 2.07E−10 | 2.32 | 2589 | 210 | 398 | 75 |
| GO:0051252 | regulation of RNA | 8.44E−13 | 2.17E−10 | 1.5 | 2589 | 342 | 970 | 192 |
| | metabolic process | | | | | | | |
| GO:0051173 | positive regulation of | 1.09E−12 | 2.72E−10 | 1.62 | 2589 | 410 | 634 | 163 |
| | nitrogen compound | | | | | | | |
| | metabolic process | | | | | | | |
| GO:0006396 | RNA processing | 1.21E−12 | 2.95E−10 | 1.7 | 2589 | 186 | 985 | 120 |
| GO:0051172 | negative regulation of | 1.82E−12 | 4.32E−10 | 1.68 | 2589 | 324 | 675 | 142 |
| | nitrogen compound | | | | | | | |
| | metabolic process | | | | | | | |
| GO:0031324 | negative regulation of | 1.87E−12 | 4.33E−10 | 1.58 | 2589 | 364 | 755 | 168 |
| | cellular metabolic process | | | | | | | |
| GO:0034622 | cellular protein-containing | 2.29E−12 | 5.17E−10 | 1.86 | 2589 | 206 | 709 | 105 |
| | complex assembly | | | | | | | |
| GO:0009892 | negative regulation of | 5.98E−12 | 1.31E−09 | 1.53 | 2589 | 413 | 755 | 184 |
| | metabolic process | | | | | | | |
| GO:0002181 | cytoplasmic translation | 7.38E−12 | 1.58E−09 | 4.93 | 2589 | 29 | 398 | 22 |
| GO:0009893 | positive regulation of | 1.20E−11 | 2.52E−09 | 1.53 | 2589 | 494 | 634 | 185 |
| | metabolic process | | | | | | | |
| GO:0010608 | posttranscriptional | 1.30E−11 | 2.67E−09 | 2.69 | 2589 | 110 | 447 | 51 |
| | regulation of gene | | | | | | | |
| | expression | | | | | | | |
| GO:0016071 | mRNA metabolic process | 1.85E−11 | 3.71E−09 | 1.77 | 2589 | 142 | 980 | 95 |
| GO:0071840 | cellular component | 1.90E−11 | 3.73E−09 | 1.27 | 2589 | 860 | 925 | 390 |
| | organization or biogenesis | | | | | | | |
| GO:0031325 | positive regulation of | 2.21E−11 | 4.24E−09 | 1.61 | 2589 | 436 | 567 | 154 |
| | cellular metabolic process | | | | | | | |
| GO:0010628 | positive regulation of gene | 2.23E−11 | 4.18E−09 | 1.69 | 2589 | 242 | 772 | 122 |
| | expression | | | | | | | |
| GO:0071826 | ribonucleoprotein complex | 2.44E−11 | 4.49E−09 | 2.02 | 2589 | 90 | 925 | 65 |
| | subunit organization | | | | | | | |
| GO:2000278 | regulation of DNA | 3.67E−11 | 6.61E−09 | 8.47 | 2589 | 30 | 163 | 16 |
| | biosynthetic process | | | | | | | |
| GO:0022618 | ribonucleoprotein complex | 4.43E−11 | 7.83E−09 | 2.03 | 2589 | 87 | 925 | 63 |
| | assembly | | | | | | | |
| GO:0031328 | positive regulation of | 6.88E−11 | 1.19E−08 | 1.81 | 2589 | 236 | 626 | 103 |
| | cellular biosynthetic | | | | | | | |
| | process | | | | | | | |
| GO:0051130 | positive regulation of | 1.08E−10 | 1.83E−08 | 1.66 | 2589 | 227 | 817 | 119 |
| | cellular component | | | | | | | |
| | organization | | | | | | | |
| GO:0006397 | mRNA processing | 1.40E−10 | 2.33E−08 | 1.8 | 2589 | 122 | 980 | 83 |
| GO:0051128 | regulation of cellular | 1.53E−10 | 2.50E−08 | 1.46 | 2589 | 416 | 817 | 192 |
| | component organization | | | | | | | |
| GO:2000573 | positive regulation of DNA | 1.55E−10 | 2.49E−08 | 9.27 | 2589 | 24 | 163 | 14 |
| | biosynthetic process | | | | | | | |
| GO:0032204 | regulation of telomere | 1.95E−10 | 3.08E−08 | 10.45 | 2589 | 23 | 140 | 13 |
| | maintenance | | | | | | | |
| GO:0009891 | positive regulation of | 2.15E−10 | 3.35E−08 | 1.76 | 2589 | 249 | 626 | 106 |
| | biosynthetic process | | | | | | | |
| GO:0043933 | protein-containing complex | 2.39E−10 | 3.65E−08 | 1.5 | 2589 | 428 | 709 | 176 |
| | subunit organization | | | | | | | |
| GO:0051253 | negative regulation of | 2.52E−10 | 3.78E−08 | 1.98 | 2589 | 142 | 689 | 75 |
| | RNA metabolic process | | | | | | | |
| GO:0008380 | RNA splicing | 2.71E−10 | 4.00E−08 | 1.85 | 2589 | 104 | 980 | 73 |
| GO:0010629 | negative regulation of gene | 5.72E−10 | 8.31E−08 | 1.77 | 2589 | 212 | 689 | 100 |
| | expression | | | | | | | |
| GO:0016043 | cellular component | 6.22E−10 | 8.89E−08 | 1.26 | 2589 | 836 | 925 | 375 |
| | organization | | | | | | | |
| GO:0010557 | positive regulation of | 6.71E−10 | 9.45E−08 | 1.83 | 2589 | 213 | 626 | 94 |
| | macromolecule | | | | | | | |
| | biosynthetic process | | | | | | | |
| GO:0045934 | negative regulation of | 8.22E−10 | 1.14E−07 | 1.82 | 2589 | 158 | 773 | 86 |
| | nucleobase-containing | | | | | | | |
| | compound metabolic | | | | | | | |
| | process | | | | | | | |
| GO:0051052 | regulation of DNA | 9.44E−10 | 1.29E−07 | 3.38 | 2589 | 74 | 331 | 32 |
| | metabolic process | | | | | | | |
| GO:0033043 | regulation of organelle | 1.03E−09 | 1.39E−07 | 1.62 | 2589 | 233 | 817 | 119 |
| | organization | | | | | | | |
| GO:0045935 | positive regulation of | 1.37E−09 | 1.81E−07 | 1.84 | 2589 | 218 | 567 | 88 |
| | nucleobase-containing | | | | | | | |
| | compound metabolic | | | | | | | |
| | process | | | | | | | |
| GO:0034248 | regulation of cellular amide | 1.43E−09 | 1.86E−07 | 2.56 | 2589 | 104 | 447 | 46 |
| | metabolic process | | | | | | | |
| GO:1903506 | regulation of nucleic acid- | 1.94E−09 | 2.50E−07 | 1.57 | 2589 | 278 | 791 | 133 |
| | templated transcription | | | | | | | |
| GO:2001141 | regulation of RNA | 2.20E−09 | 2.80E−07 | 1.56 | 2589 | 281 | 791 | 134 |
| | biosynthetic process | | | | | | | |
| GO:1904851 | positive regulation of | 2.42E−09 | 3.03E−07 | 16.68 | 2589 | 9 | 138 | 8 |
| | establishment of protein | | | | | | | |
| | localization to telomere | | | | | | | |
| GO:1904816 | positive regulation of | 2.42E−09 | 2.99E−07 | 16.68 | 2589 | 9 | 138 | 8 |
| | protein localization to | | | | | | | |
| | chromosome, telomeric | | | | | | | |
| | region | | | | | | | |
| GO:0070203 | regulation of establishment | 2.42E−09 | 2.95E−07 | 16.68 | 2589 | 9 | 138 | 8 |
| | of protein localization to | | | | | | | |
| | telomere | | | | | | | |
| GO:0070202 | regulation of establishment | 2.42E−09 | 2.91E−07 | 16.68 | 2589 | 9 | 138 | 8 |
| | of protein localization to | | | | | | | |
| | chromosome | | | | | | | |
| GO:0032880 | regulation of protein | 2.79E−09 | 3.31E−07 | 2.04 | 2589 | 192 | 469 | 71 |
| | localization | | | | | | | |
| GO:0022607 | cellular component | 4.23E−09 | 4.95E−07 | 1.42 | 2589 | 503 | 709 | 196 |
| | assembly | | | | | | | |
| GO:0033044 | regulation of chromosome | 4.37E−09 | 5.05E−07 | 5.94 | 2589 | 56 | 140 | 18 |
| | organization | | | | | | | |
| GO:0009890 | negative regulation of | 6.51E−09 | 7.43E−07 | 2.39 | 2589 | 182 | 309 | 52 |
| | biosynthetic process | | | | | | | |
| GO:0032101 | regulation of response to | 6.63E−09 | 7.46E−07 | 6.96 | 2589 | 96 | 62 | 16 |
| | external stimulus | | | | | | | |
| GO:0032206 | positive regulation of | 8.00E−09 | 8.90E−07 | 11.73 | 2589 | 16 | 138 | 10 |
| | telomere maintenance | | | | | | | |
| GO:0006355 | regulation of transcription, | 8.68E−09 | 9.54E−07 | 1.55 | 2589 | 275 | 791 | 130 |
| | DNA-templated | | | | | | | |
| GO:1904356 | regulation of telomere | 9.39E−09 | 1.02E−06 | 10.32 | 2589 | 20 | 138 | 11 |
| | maintenance via telomere | | | | | | | |
| | lengthening | | | | | | | |
| GO:0006417 | regulation of translation | 9.64E−09 | 1.03E−06 | 2.56 | 2589 | 95 | 447 | 42 |
| GO:0080134 | regulation of response to | 1.00E−08 | 1.06E−06 | 1.93 | 2589 | 196 | 512 | 75 |
| | stress | | | | | | | |
| GO:1901998 | toxin transport | 1.27E−08 | 1.33E−06 | 12.99 | 2589 | 13 | 138 | 9 |
| GO:0065003 | protein-containing complex | 1.33E−08 | 1.38E−06 | 1.48 | 2589 | 396 | 709 | 160 |
| | assembly | | | | | | | |
| GO:1904814 | regulation of protein | 1.38E−08 | 1.41E−06 | 15.01 | 2589 | 10 | 138 | 8 |
| | localization to | | | | | | | |
| | chromosome, telomeric | | | | | | | |
| | region | | | | | | | |
| GO:0008037 | cell recognition | 1.38E−08 | 1.40E−06 | 8.77 | 2589 | 23 | 154 | 12 |
| GO:0031327 | negative regulation of | 1.60E−08 | 1.60E−06 | 2.42 | 2589 | 170 | 309 | 49 |
| | cellular biosynthetic | | | | | | | |
| | process | | | | | | | |
| GO:1904951 | positive regulation of | 2.03E−08 | 2.01E−06 | 2.49 | 2589 | 93 | 469 | 42 |
| | establishment of protein | | | | | | | |
| | localization | | | | | | | |
| GO:0031349 | positive regulation of | 2.05E−08 | 2.01E−06 | 9.63 | 2589 | 43 | 75 | 12 |
| | defense response | | | | | | | |
| GO:0035036 | sperm-egg recognition | 2.14E−08 | 2.07E−06 | 5.9 | 2589 | 11 | 439 | 11 |
| GO:0007339 | binding of sperm to zona | 2.14E−08 | 2.05E−06 | 5.9 | 2589 | 11 | 439 | 11 |
| | pellucida | | | | | | | |
| GO:0048583 | regulation of response to | 2.33E−08 | 2.21E−06 | 1.46 | 2589 | 443 | 655 | 164 |
| | stimulus | | | | | | | |
| GO:0050729 | positive regulation of | 3.01E−08 | 2.83E−06 | 18.56 | 2589 | 18 | 62 | 8 |
| | inflammatory response | | | | | | | |
| GO:0010638 | positive regulation of | 4.01E−08 | 3.72E−06 | 1.82 | 2589 | 122 | 817 | 70 |
| | organelle organization | | | | | | | |
| GO:0051054 | positive regulation of DNA | 4.15E−08 | 3.81E−06 | 3.75 | 2589 | 48 | 331 | 23 |
| | metabolic process | | | | | | | |
| GO:0070201 | regulation of establishment | 4.59E−08 | 4.17E−06 | 2.45 | 2589 | 143 | 333 | 45 |
| | of protein localization | | | | | | | |
| GO:0051247 | positive regulation of | 4.68E−08 | 4.22E−06 | 1.67 | 2589 | 240 | 634 | 98 |
| | protein metabolic process | | | | | | | |
| GO:0032210 | regulation of telomere | 4.75E−08 | 4.24E−06 | 10.42 | 2589 | 18 | 138 | 10 |
| | maintenance via telomerase | | | | | | | |
| GO:0031647 | regulation of protein | 4.82E−08 | 4.26E−06 | 4.65 | 2589 | 71 | 157 | 20 |
| | stability | | | | | | | |
| GO:0032879 | regulation of localization | 4.93E−08 | 4.31E−06 | 2.18 | 2589 | 399 | 161 | 54 |
| GO:2000113 | negative regulation of | 5.23E−08 | 4.53E−06 | 2.45 | 2589 | 154 | 309 | 45 |
| | cellular macromolecule | | | | | | | |
| | biosynthetic process | | | | | | | |
| GO:2001252 | positive regulation of | 5.32E−08 | 4.57E−06 | 6.81 | 2589 | 38 | 140 | 14 |
| | chromosome organization | | | | | | | |
| GO:0010558 | negative regulation of | 6.12E−08 | 5.20E−06 | 1.8 | 2589 | 161 | 689 | 77 |
| | macromolecule | | | | | | | |
| | biosynthetic process | | | | | | | |
| GO:0050727 | regulation of inflammatory | 6.14E−08 | 5.17E−06 | 10.21 | 2589 | 45 | 62 | 11 |
| | response | | | | | | | |
| GO:0051716 | cellular response to | 6.47E−08 | 5.40E−06 | 1.55 | 2589 | 428 | 498 | 128 |
| | stimulus | | | | | | | |
| GO:0044419 | interspecies interaction | 8.49E−08 | 7.02E−06 | 2.06 | 2589 | 73 | 810 | 47 |
| | between organisms | | | | | | | |
| GO:1904358 | positive regulation of | 9.82E−08 | 8.04E−06 | 11.26 | 2589 | 15 | 138 | 9 |
| | telomere maintenance via | | | | | | | |
| | telomere lengthening | | | | | | | |
| GO:1903507 | negative regulation of | 1.01E−07 | 8.24E−06 | 1.96 | 2589 | 111 | 689 | 58 |
| | nucleic acid-templated | | | | | | | |
| | transcription | | | | | | | |
| GO:0070887 | cellular response to | 1.04E−07 | 8.33E−06 | 1.7 | 2589 | 285 | 497 | 93 |
| | chemical stimulus | | | | | | | |
| GO:0065009 | regulation of molecular | 1.08E−07 | 8.63E−06 | 1.99 | 2589 | 369 | 226 | 64 |
| | function | | | | | | | |
| GO:0032502 | developmental process | 1.39E−07 | 1.10E−05 | 2.56 | 2589 | 545 | 63 | 34 |
| GO:0032103 | positive regulation of | 1.42E−07 | 1.11E−05 | 10.99 | 2589 | 38 | 62 | 10 |
| | response to external | | | | | | | |
| | stimulus | | | | | | | |
| GO:0031347 | regulation of defense | 1.46E−07 | 1.13E−05 | 6.16 | 2589 | 84 | 75 | 15 |
| | response | | | | | | | |
| GO:0006259 | DNA metabolic process | 1.57E−07 | 1.21E−05 | 1.79 | 2589 | 86 | 991 | 59 |
| GO:1902679 | negative regulation of | 1.57E−07 | 1.20E−05 | 1.95 | 2589 | 112 | 689 | 58 |
| | RNA biosynthetic process | | | | | | | |
| GO:0032270 | positive regulation of | 1.80E−07 | 1.36E−05 | 2.25 | 2589 | 219 | 252 | 48 |
| | cellular protein metabolic | | | | | | | |
| | process | | | | | | | |
| GO:0050896 | response to stimulus | 1.85E−07 | 1.39E−05 | 1.4 | 2589 | 752 | 441 | 179 |
| GO:0045892 | negative regulation of | 2.03E−07 | 1.51E−05 | 1.95 | 2589 | 110 | 689 | 57 |
| | transcription, DNA- | | | | | | | |
| | templated | | | | | | | |
| GO:0051049 | regulation of transport | 2.13E−07 | 1.58E−05 | 3.43 | 2589 | 292 | 62 | 24 |
| GO:0006954 | inflammatory response | 2.16E−07 | 1.58E−05 | 9.04 | 2589 | 42 | 75 | 11 |
| GO:0050793 | regulation of | 2.19E−07 | 1.59E−05 | 1.7 | 2589 | 323 | 429 | 91 |
| | developmental process | | | | | | | |
| GO:0071345 | cellular response to | 2.46E−07 | 1.77E−05 | 2.29 | 2589 | 98 | 497 | 43 |
| | cytokine stimulus | | | | | | | |
| GO:0030029 | actin filament-based | 2.51E−07 | 1.79E−05 | 2.37 | 2589 | 74 | 561 | 38 |
| | process | | | | | | | |
| GO:0034097 | response to cytokine | 2.62E−07 | 1.86E−05 | 2.1 | 2589 | 120 | 523 | 51 |
| GO:0065008 | regulation of biological | 2.85E−07 | 2.00E−05 | 1.83 | 2589 | 614 | 159 | 69 |
| | quality | | | | | | | |
| GO:0007010 | cytoskeleton organization | 3.04E−07 | 2.12E−05 | 2.01 | 2589 | 126 | 561 | 55 |
| GO:1902903 | regulation of | 3.06E−07 | 2.12E−05 | 2.13 | 2589 | 73 | 717 | 43 |
| | supramolecular fiber | | | | | | | |
| | organization | | | | | | | |
| GO:0071310 | cellular response to organic | 3.10E−07 | 2.13E−05 | 1.75 | 2589 | 228 | 512 | 79 |
| | substance | | | | | | | |
| GO:1901566 | organonitrogen compound | 4.14E−07 | 2.83E−05 | 1.7 | 2589 | 356 | 381 | 89 |
| | biosynthetic process | | | | | | | |
| GO:1900046 | regulation of hemostasis | 4.19E−07 | 2.84E−05 | 3.85 | 2589 | 21 | 512 | 16 |
| GO:0030193 | regulation of blood | 4.19E−07 | 2.82E−05 | 3.85 | 2589 | 21 | 512 | 16 |
| | coagulation | | | | | | | |
| GO:0050818 | regulation of coagulation | 4.19E−07 | 2.80E−05 | 3.85 | 2589 | 21 | 512 | 16 |
| GO:0009988 | cell-cell recognition | 4.64E−07 | 3.07E−05 | 11.55 | 2589 | 13 | 138 | 8 |
| GO:0006325 | chromatin organization | 4.81E−07 | 3.16E−05 | 1.78 | 2589 | 82 | 991 | 56 |
| GO:0010639 | negative regulation of | 4.96E−07 | 3.24E−05 | 2.1 | 2589 | 78 | 696 | 44 |
| | organelle organization | | | | | | | |
| GO:0061041 | regulation of wound | 5.27E−07 | 3.41E−05 | 3.43 | 2589 | 28 | 512 | 19 |
| | healing | | | | | | | |
| GO:0048584 | positive regulation of | 5.35E−07 | 3.45E−05 | 1.61 | 2589 | 262 | 602 | 98 |
| | response to stimulus | | | | | | | |
| GO:0032271 | regulation of protein | 6.65E−07 | 4.25E−05 | 2.24 | 2589 | 58 | 717 | 36 |
| | polymerization | | | | | | | |
| GO:0042274 | ribosomal small subunit | 8.87E−07 | 5.63E−05 | 11.85 | 2589 | 9 | 170 | 7 |
| | biogenesis | | | | | | | |
| GO:0050707 | regulation of cytokine | 8.88E−07 | 5.60E−05 | 5.15 | 2589 | 21 | 311 | 13 |
| | secretion | | | | | | | |
| GO:0044271 | cellular nitrogen compound | 9.28E−07 | 5.81E−05 | 1.66 | 2589 | 373 | 381 | 91 |
| | biosynthetic process | | | | | | | |
| GO:0022402 | cell cycle process | 9.48E−07 | 5.89E−05 | 1.85 | 2589 | 95 | 810 | 55 |
| GO:1902904 | negative regulation of | 9.56E−07 | 5.90E−05 | 2.91 | 2589 | 36 | 568 | 23 |
| | supramolecular fiber | | | | | | | |
| | organization | | | | | | | |
| GO:0051494 | negative regulation of | 9.56E−07 | 5.86E−05 | 2.91 | 2589 | 36 | 568 | 23 |
| | cytoskeleton organization | | | | | | | |
| GO:0051248 | negative regulation of | 1.01E−06 | 6.18E−05 | 1.8 | 2589 | 199 | 513 | 71 |
| | protein metabolic process | | | | | | | |
| GO:0050790 | regulation of catalytic | 1.04E−06 | 6.27E−05 | 2.06 | 2589 | 289 | 226 | 52 |
| | activity | | | | | | | |
| GO:0002376 | immune system process | 1.09E−06 | 6.55E−05 | 1.93 | 2589 | 185 | 434 | 60 |
| GO:0032212 | positive regulation of | 1.09E−06 | 6.52E−05 | 4.52 | 2589 | 14 | 491 | 12 |
| | telomere maintenance via | | | | | | | |
| | telomerase | | | | | | | |
| GO:0061013 | regulation of mRNA | 1.37E−06 | 8.12E−05 | 3.63 | 2589 | 31 | 414 | 18 |
| | catabolic process | | | | | | | |
| GO:0000028 | ribosomal small subunit | 1.40E−06 | 8.27E−05 | 9.1 | 2589 | 11 | 207 | 8 |
| | assembly | | | | | | | |
| GO:0030162 | regulation of proteolysis | 1.55E−06 | 9.09E−05 | 1.96 | 2589 | 146 | 497 | 55 |
| GO:0051050 | positive regulation of | 1.63E−06 | 9.50E−05 | 4.25 | 2589 | 177 | 62 | 18 |
| | transport | | | | | | | |
| GO:1903312 | negative regulation of | 1.66E−06 | 9.56E−05 | 5.5 | 2589 | 30 | 204 | 13 |
| | mRNA metabolic process | | | | | | | |
| GO:1903827 | regulation of cellular | 1.70E−06 | 9.75E−05 | 2.07 | 2589 | 124 | 484 | 48 |
| | protein localization | | | | | | | |
| GO:0051972 | regulation of telomerase | 1.80E−06 | 1.03E−04 | 6.02 | 2589 | 13 | 331 | 10 |
| | activity | | | | | | | |
| GO:0051973 | positive regulation of | 1.80E−06 | 1.02E−04 | 6.02 | 2589 | 13 | 331 | 10 |
| | telomerase activity | | | | | | | |
| GO:0051239 | regulation of multicellular | 1.88E−06 | 1.06E−04 | 2.87 | 2589 | 378 | 62 | 26 |
| | organismal process | | | | | | | |
| GO:0045595 | regulation of cell | 1.95E−06 | 1.09E−04 | 1.74 | 2589 | 218 | 491 | 72 |
| | differentiation | | | | | | | |
| GO:0000377 | RNA splicing, via | 2.11E−06 | 1.17E−04 | 1.84 | 2589 | 66 | 980 | 46 |
| | transesterification reactions | | | | | | | |
| | with bulged adenosine as | | | | | | | |
| | nucleophile | | | | | | | |
| GO:0000375 | RNA splicing, via | 2.11E−06 | 1.17E−04 | 1.84 | 2589 | 66 | 980 | 46 |
| | transesterification reactions | | | | | | | |
| GO:0000398 | mRNA splicing, via | 2.11E−06 | 1.16E−04 | 1.84 | 2589 | 66 | 980 | 46 |
| | spliceosome | | | | | | | |
| GO:0006334 | nucleosome assembly | 2.34E−06 | 1.28E−04 | 2.66 | 2589 | 24 | 811 | 20 |
| GO:0009894 | regulation of catabolic | 2.35E−06 | 1.28E−04 | 1.83 | 2589 | 184 | 501 | 65 |
| | process | | | | | | | |
| GO:0043488 | regulation of mRNA | 2.40E−06 | 1.30E−04 | 3.67 | 2589 | 29 | 414 | 17 |
| | stability | | | | | | | |
| GO:0034728 | nucleosome organization | 2.76E−06 | 1.48E−04 | 2.47 | 2589 | 29 | 830 | 23 |
| GO:0090087 | regulation of peptide | 2.85E−06 | 1.52E−04 | 2.29 | 2589 | 136 | 333 | 40 |
| | transport | | | | | | | |
| GO:0051129 | negative regulation of | 2.88E−06 | 1.53E−04 | 1.78 | 2589 | 136 | 673 | 63 |
| | cellular component | | | | | | | |
| | organization | | | | | | | |
| GO:1903829 | positive regulation of | 2.98E−06 | 1.57E−04 | 2.77 | 2589 | 82 | 319 | 28 |
| | cellular protein localization | | | | | | | |
| GO:0015669 | gas transport | 3.12E−06 | 1.63E−04 | 129.45 | 2589 | 6 | 10 | 3 |
| GO:0050821 | protein stabilization | 3.28E−06 | 1.71E−04 | 4.76 | 2589 | 52 | 157 | 15 |
| GO:0032269 | negative regulation of | 3.28E−06 | 1.70E−04 | 1.79 | 2589 | 189 | 513 | 67 |
| | cellular protein metabolic | | | | | | | |
| | process | | | | | | | |
| GO:0006357 | regulation of transcription | 3.30E−06 | 1.70E−04 | 1.59 | 2589 | 177 | 789 | 86 |
| | by RNA polymerase II | | | | | | | |
| GO:0002697 | regulation of immune | 3.59E−06 | 1.84E−04 | 7.07 | 2589 | 61 | 66 | 11 |
| | effector process | | | | | | | |
| GO:1903034 | regulation of response to | 3.73E−06 | 1.90E−04 | 3.06 | 2589 | 33 | 512 | 20 |
| | wounding | | | | | | | |
| GO:0051704 | multi-organism process | 3.74E−06 | 1.89E−04 | 1.68 | 2589 | 165 | 690 | 74 |
| GO:0043254 | regulation of protein | 3.83E−06 | 1.93E−04 | 2 | 2589 | 105 | 580 | 47 |
| | complex assembly | | | | | | | |
| GO:1900047 | negative regulation of | 4.06E−06 | 2.03E−04 | 5.8 | 2589 | 14 | 319 | 10 |
| | hemostasis | | | | | | | |
| GO:0030195 | negative regulation of | 4.06E−06 | 2.02E−04 | 5.8 | 2589 | 14 | 319 | 10 |
| | blood coagulation | | | | | | | |
| GO:0050819 | negative regulation of | 4.06E−06 | 2.01E−04 | 5.8 | 2589 | 14 | 319 | 10 |
| | coagulation | | | | | | | |
| GO:0010033 | response to organic | 4.06E−06 | 2.00E−04 | 2.73 | 2589 | 371 | 69 | 27 |
| | substance | | | | | | | |
| GO:0051493 | regulation of cytoskeleton | 4.08E−06 | 2.00E−04 | 1.74 | 2589 | 112 | 810 | 61 |
| | organization | | | | | | | |
| GO:0043603 | cellular amide metabolic | 4.12E−06 | 2.01E−04 | 1.7 | 2589 | 295 | 398 | 77 |
| | process | | | | | | | |
| GO:0006952 | defense response | 4.49E−06 | 2.18E−04 | 1.98 | 2589 | 131 | 500 | 50 |
| GO:0043487 | regulation of RNA stability | 4.57E−06 | 2.20E−04 | 3.31 | 2589 | 30 | 469 | 18 |
| GO:0051241 | negative regulation of | 4.66E−06 | 2.23E−04 | 2.11 | 2589 | 140 | 395 | 45 |
| | multicellular organismal | | | | | | | |
| | process | | | | | | | |
| GO:0030036 | actin cytoskeleton | 4.78E−06 | 2.28E−04 | 2.31 | 2589 | 66 | 561 | 33 |
| | organization | | | | | | | |
| GO:0002682 | regulation of immune | 4.88E−06 | 2.32E−04 | 1.93 | 2589 | 173 | 427 | 55 |
| | system process | | | | | | | |
| GO:1902369 | negative regulation of | 4.92E−06 | 2.32E−04 | 3.04 | 2589 | 19 | 717 | 16 |
| | RNA catabolic process | | | | | | | |
| GO:0032272 | negative regulation of | 5.00E−06 | 2.35E−04 | 2.58 | 2589 | 27 | 779 | 21 |
| | protein polymerization | | | | | | | |
| GO:0006139 | nucleobase-containing | 6.19E−06 | 2.89E−04 | 1.24 | 2589 | 550 | 992 | 262 |
| | compound metabolic | | | | | | | |
| | process | | | | | | | |
| GO:0031329 | regulation of cellular | 6.45E−06 | 3.00E−04 | 1.9 | 2589 | 160 | 469 | 55 |
| | catabolic process | | | | | | | |
| GO:1902533 | positive regulation of | 6.55E−06 | 3.03E−04 | 2.83 | 2589 | 114 | 217 | 27 |
| | intracellular signal | | | | | | | |
| | transduction | | | | | | | |
| GO:0050710 | negative regulation of | 6.59E−06 | 3.03E−04 | 8 | 2589 | 8 | 283 | 7 |
| | cytokine secretion | | | | | | | |
| GO:0043489 | RNA stabilization | 6.76E−06 | 3.09E−04 | 2.82 | 2589 | 16 | 862 | 15 |
| GO:1902373 | negative regulation of | 6.76E−06 | 3.08E−04 | 7.14 | 2589 | 16 | 204 | 9 |
| | mRNA catabolic process | | | | | | | |
| GO:0022414 | reproductive process | 6.81E−06 | 3.09E−04 | 1.67 | 2589 | 126 | 824 | 67 |
| GO:0045727 | positive regulation of | 7.73E−06 | 3.48E−04 | 2.68 | 2589 | 34 | 626 | 22 |
| | translation | | | | | | | |
| GO:0044093 | positive regulation of | 7.75E−06 | 3.48E−04 | 3.79 | 2589 | 232 | 50 | 17 |
| | molecular function | | | | | | | |
| GO:0002819 | regulation of adaptive | 7.76E−06 | 3.46E−04 | 12.48 | 2589 | 22 | 66 | 7 |
| | immune response | | | | | | | |
| GO:0098760 | response to interleukin-7 | 8.26E−06 | 3.67E−04 | 8.31 | 2589 | 14 | 178 | 8 |
| GO:0098761 | cellular response to | 8.26E−06 | 3.65E−04 | 8.31 | 2589 | 14 | 178 | 8 |
| | interleukin-7 | | | | | | | |
| GO:0110053 | regulation of actin filament | 8.33E−06 | 3.66E−04 | 1.92 | 2589 | 58 | 908 | 39 |
| | organization | | | | | | | |
| GO:0051254 | positive regulation of RNA | 8.55E−06 | 3.74E−04 | 1.48 | 2589 | 175 | 980 | 98 |
| | metabolic process | | | | | | | |
| GO:0006413 | translational initiation | 8.68E−06 | 3.78E−04 | 2.27 | 2589 | 33 | 863 | 25 |
| GO:0051240 | positive regulation of | 8.89E−06 | 3.85E−04 | 3.45 | 2589 | 230 | 62 | 19 |
| | multicellular organismal | | | | | | | |
| | process | | | | | | | |
| GO:0009987 | cellular process | 9.10E−06 | 3.92E−04 | 1.08 | 2589 | 2086 | 679 | 590 |
| GO:0002791 | regulation of peptide | 9.13E−06 | 3.92E−04 | 6.48 | 2589 | 72 | 61 | 11 |
| | secretion | | | | | | | |
| GO:0006996 | organelle organization | 9.31E−06 | 3.98E−04 | 1.3 | 2589 | 380 | 991 | 189 |
| GO:0071824 | protein-DNA complex | 9.32E−06 | 3.96E−04 | 2.34 | 2589 | 32 | 830 | 24 |
| | subunit organization | | | | | | | |
| GO:2001020 | regulation of response to | 1.07E−05 | 4.51E−04 | 4.16 | 2589 | 28 | 311 | 14 |
| | DNA damage stimulus | | | | | | | |
| GO:0044265 | cellular macromolecule | 1.08E−05 | 4.55E−04 | 1.64 | 2589 | 126 | 852 | 68 |
| | catabolic process | | | | | | | |
| GO:0006511 | ubiquitin-dependent | 1.14E−05 | 4.77E−04 | 1.79 | 2589 | 86 | 841 | 50 |
| | protein catabolic process | | | | | | | |
| GO:0034250 | positive regulation of | 1.24E−05 | 5.19E−04 | 2.57 | 2589 | 37 | 626 | 23 |
| | cellular amide metabolic | | | | | | | |
| | process | | | | | | | |
| GO:0022604 | regulation of cell | 1.38E−05 | 5.74E−04 | 2.35 | 2589 | 83 | 425 | 32 |
| | morphogenesis | | | | | | | |
| GO:0002821 | positive regulation of | 1.39E−05 | 5.75E−04 | 14.71 | 2589 | 16 | 66 | 6 |
| | adaptive immune response | | | | | | | |
| GO:0031399 | regulation of protein | 1.45E−05 | 5.96E−04 | 2.22 | 2589 | 218 | 198 | 37 |
| | modification process | | | | | | | |
| GO:0045597 | positive regulation of cell | 1.59E−05 | 6.53E−04 | 2.88 | 2589 | 134 | 168 | 25 |
| | differentiation | | | | | | | |
| GO:0016072 | rRNA metabolic process | 1.64E−05 | 6.70E−04 | 3.19 | 2589 | 57 | 285 | 20 |
| GO:1903311 | regulation of mRNA | 1.67E−05 | 6.77E−04 | 1.75 | 2589 | 71 | 980 | 47 |
| | metabolic process | | | | | | | |
| GO:0010646 | regulation of cell | 1.68E−05 | 6.80E−04 | 2.57 | 2589 | 362 | 75 | 27 |
| | communication | | | | | | | |
| GO:0044087 | regulation of cellular | 1.71E−05 | 6.87E−04 | 1.49 | 2589 | 169 | 946 | 92 |
| | component biogenesis | | | | | | | |
| GO:0051222 | positive regulation of | 1.72E−05 | 6.91E−04 | 2.28 | 2589 | 83 | 452 | 33 |
| | protein transport | | | | | | | |
| GO:0009966 | regulation of signal | 1.82E−05 | 7.24E−04 | 2.11 | 2589 | 312 | 157 | 40 |
| | transduction | | | | | | | |
| GO:0048255 | mRNA stabilization | 1.85E−05 | 7.36E−04 | 2.8 | 2589 | 15 | 862 | 14 |
| GO:0016032 | viral process | 1.95E−05 | 7.72E−04 | 2.25 | 2589 | 32 | 863 | 24 |
| GO:0044403 | symbiont process | 1.95E−05 | 7.68E−04 | 2.25 | 2589 | 32 | 863 | 24 |
| GO:0042981 | regulation of apoptotic | 2.14E−05 | 8.37E−04 | 2.28 | 2589 | 249 | 155 | 34 |
| | process | | | | | | | |
| GO:0008064 | regulation of actin | 2.21E−05 | 8.60E−04 | 2.17 | 2589 | 50 | 717 | 30 |
| | polymerization or | | | | | | | |
| | depolymerization | | | | | | | |
| GO:0023051 | regulation of signaling | 2.31E−05 | 8.99E−04 | 2.53 | 2589 | 368 | 75 | 27 |
| GO:0051223 | regulation of protein | 2.48E−05 | 9.57E−04 | 2.2 | 2589 | 131 | 333 | 37 |
| | transport | | | | | | | |
| GO:0065004 | protein-DNA complex | 2.48E−05 | 9.57E−04 | 2.46 | 2589 | 26 | 811 | 20 |
| | assembly | | | | | | | |
| GO:0030833 | regulation of actin filament | 2.49E−05 | 9.54E−04 | 1.98 | 2589 | 46 | 908 | 32 |
| | polymerization | | | | | | | |
| GO:0006457 | protein folding | 2.49E−05 | 9.50E−04 | 3.36 | 2589 | 77 | 190 | 19 |
| GO:1900180 | regulation of protein | 2.54E−05 | 9.67E−04 | 3.92 | 2589 | 29 | 319 | 14 |
| | localization to nucleus | | | | | | | |
| GO:0006950 | response to stress | 2.59E−05 | 9.81E−04 | 2.31 | 2589 | 423 | 82 | 31 |
| GO:0006364 | rRNA processing | 2.87E−05 | 1.08E−03 | 3.2 | 2589 | 54 | 285 | 19 |
| GO:0030834 | regulation of actin filament | 3.09E−05 | 1.16E−03 | 2.53 | 2589 | 25 | 779 | 19 |
| | depolymerization | | | | | | | |
| GO:0015671 | oxygen transport | 3.11E−05 | 1.16E−03 | 323.62 | 2589 | 4 | 4 | 2 |
| GO:0043067 | regulation of programmed | 3.12E−05 | 1.16E−03 | 2.24 | 2589 | 253 | 155 | 34 |
| | cell death | | | | | | | |
| GO:0019220 | regulation of phosphate | 3.15E−05 | 1.17E−03 | 1.44 | 2589 | 224 | 825 | 103 |
| | metabolic process | | | | | | | |
| GO:0051174 | regulation of phosphorus | 3.15E−05 | 1.16E−03 | 1.44 | 2589 | 224 | 825 | 103 |
| | metabolic process | | | | | | | |
| GO:0006919 | activation of cysteine-type | 3.17E−05 | 1.16E−03 | 19.26 | 2589 | 16 | 42 | 5 |
| | endopeptidase activity | | | | | | | |
| | involved in apoptotic | | | | | | | |
| | process | | | | | | | |
| GO:0010941 | regulation of cell death | 3.35E−05 | 1.23E−03 | 2.15 | 2589 | 286 | 156 | 37 |
| GO:0090303 | positive regulation of | 3.36E−05 | 1.22E−03 | 3.04 | 2589 | 11 | 851 | 11 |
| | wound healing | | | | | | | |
| GO:0010564 | regulation of cell cycle | 3.46E−05 | 1.26E−03 | 1.83 | 2589 | 84 | 757 | 45 |
| | process | | | | | | | |
| GO:0006807 | nitrogen compound | 3.49E−05 | 1.26E−03 | 1.16 | 2589 | 1255 | 671 | 378 |
| | metabolic process | | | | | | | |
| GO:0052547 | regulation of peptidase | 3.50E−05 | 1.26E−03 | 2.95 | 2589 | 87 | 222 | 22 |
| | activity | | | | | | | |
| GO:0030832 | regulation of actin filament | 3.87E−05 | 1.39E−03 | 2.12 | 2589 | 51 | 717 | 30 |
| | length | | | | | | | |
| GO:0019941 | modification-dependent | 3.97E−05 | 1.42E−03 | 1.73 | 2589 | 91 | 841 | 51 |
| | protein catabolic process | | | | | | | |
| GO:0034641 | cellular nitrogen compound | 4.03E−05 | 1.44E−03 | 1.18 | 2589 | 775 | 992 | 350 |
| | metabolic process | | | | | | | |
| GO:0051693 | actin filament capping | 4.17E−05 | 1.48E−03 | 2.77 | 2589 | 18 | 779 | 15 |
| GO:0051276 | chromosome organization | 4.20E−05 | 1.48E−03 | 1.82 | 2589 | 53 | 991 | 37 |
| GO:0000122 | negative regulation of | 4.76E−05 | 1.68E−03 | 1.99 | 2589 | 68 | 689 | 36 |
| | transcription by RNA | | | | | | | |
| | polymerase II | | | | | | | |
| GO:0034114 | regulation of heterotypic | 5.20E−05 | 1.82E−03 | 11.01 | 2589 | 6 | 196 | 5 |
| | cell-cell adhesion | | | | | | | |
| GO:2001233 | regulation of apoptotic | 5.25E−05 | 1.83E−03 | 2.25 | 2589 | 81 | 441 | 31 |
| | signaling pathway | | | | | | | |
| GO:0050776 | regulation of immune | 5.41E−05 | 1.88E−03 | 4.24 | 2589 | 114 | 75 | 14 |
| | response | | | | | | | |
| GO:0043085 | positive regulation of | 5.41E−05 | 1.87E−03 | 4.19 | 2589 | 173 | 50 | 14 |
| | catalytic activity | | | | | | | |
| GO:0030835 | negative regulation of actin | 5.52E−05 | 1.91E−03 | 2.66 | 2589 | 20 | 779 | 16 |
| | filament depolymerization | | | | | | | |
| GO:0010647 | positive regulation of cell | 5.53E−05 | 1.90E−03 | 2.41 | 2589 | 205 | 157 | 30 |
| | communication | | | | | | | |
| GO:0050878 | regulation of body fluid | 5.60E−05 | 1.92E−03 | 4.27 | 2589 | 40 | 197 | 13 |
| | levels | | | | | | | |
| GO:0006333 | chromatin assembly or | 5.88E−05 | 2.01E−03 | 4.52 | 2589 | 15 | 382 | 10 |
| | disassembly | | | | | | | |
| GO:0061045 | negative regulation of | 5.91E−05 | 2.01E−03 | 4.77 | 2589 | 17 | 319 | 10 |
| | wound healing | | | | | | | |
| GO:0002822 | regulation of adaptive | 6.53E−05 | 2.21E−03 | 11.77 | 2589 | 20 | 66 | 6 |
| | immune response based on | | | | | | | |
| | somatic recombination of | | | | | | | |
| | immune receptors built | | | | | | | |
| | from immunoglobulin | | | | | | | |
| | superfamily domains | | | | | | | |
| GO:0042325 | regulation of | 6.57E−05 | 2.22E−03 | 1.48 | 2589 | 194 | 820 | 91 |
| | phosphorylation | | | | | | | |
| GO:1901880 | negative regulation of | 6.82E−05 | 2.29E−03 | 2.57 | 2589 | 22 | 779 | 17 |
| | protein depolymerization | | | | | | | |
| GO:1901879 | regulation of protein | 6.93E−05 | 2.32E−03 | 2.87 | 2589 | 28 | 548 | 17 |
| | depolymerization | | | | | | | |
| GO:0071156 | regulation of cell cycle | 7.06E−05 | 2.36E−03 | 11.78 | 2589 | 7 | 157 | 5 |
| | arrest | | | | | | | |
| GO:0051258 | protein polymerization | 7.08E−05 | 2.35E−03 | 2.7 | 2589 | 14 | 890 | 13 |
| GO:0032200 | telomere organization | 7.37E−05 | 2.44E−03 | 4.06 | 2589 | 13 | 491 | 10 |
| GO:0000723 | telomere maintenance | 7.37E−05 | 2.43E−03 | 4.06 | 2589 | 13 | 491 | 10 |
| GO:0006281 | DNA repair | 7.64E−05 | 2.51E−03 | 1.79 | 2589 | 54 | 989 | 37 |
| GO:0023056 | positive regulation of | 7.87E−05 | 2.58E−03 | 2.38 | 2589 | 208 | 157 | 30 |
| | signaling | | | | | | | |
| GO:0018193 | peptidyl-amino acid | 7.96E−05 | 2.60E−03 | 1.7 | 2589 | 98 | 810 | 52 |
| | modification | | | | | | | |
| GO:0031935 | regulation of chromatin | 8.22E−05 | 2.68E−03 | 7.18 | 2589 | 7 | 309 | 6 |
| | silencing | | | | | | | |
| GO:0070934 | CRD-mediated mRNA | 8.59E−05 | 2.78E−03 | 8.32 | 2589 | 5 | 311 | 5 |
| | stabilization | | | | | | | |
| GO:0031333 | negative regulation of | 8.94E−05 | 2.89E−03 | 2.52 | 2589 | 38 | 568 | 21 |
| | protein complex assembly | | | | | | | |
| GO:0050764 | regulation of phagocytosis | 9.89E−05 | 3.18E−03 | 7.7 | 2589 | 15 | 157 | 7 |
| GO:0051046 | regulation of secretion | 1.00E−04 | 3.22E−03 | 4.67 | 2589 | 109 | 61 | 12 |
| GO:0071383 | cellular response to steroid | 1.02E−04 | 3.24E−03 | 6.05 | 2589 | 9 | 333 | 7 |
| | hormone stimulus | | | | | | | |
| GO:1902680 | positive regulation of RNA | 1.04E−04 | 3.32E−03 | 1.57 | 2589 | 148 | 782 | 70 |
| | biosynthetic process | | | | | | | |
| GO:0045893 | positive regulation of | 1.04E−04 | 3.31E−03 | 1.57 | 2589 | 148 | 782 | 70 |
| | transcription, DNA- | | | | | | | |
| | templated | | | | | | | |
| GO:1903508 | positive regulation of | 1.04E−04 | 3.30E−03 | 1.57 | 2589 | 148 | 782 | 70 |
| | nucleic acid-templated | | | | | | | |
| | transcription | | | | | | | |
| GO:1903036 | positive regulation of | 1.06E−04 | 3.33E−03 | 3.89 | 2589 | 13 | 512 | 10 |
| | response to wounding | | | | | | | |
| GO:2000026 | regulation of multicellular | 1.06E−04 | 3.32E−03 | 1.65 | 2589 | 241 | 417 | 64 |
| | organismal development | | | | | | | |
| GO:0006338 | chromatin remodeling | 1.06E−04 | 3.32E−03 | 2.24 | 2589 | 21 | 991 | 18 |
| GO:0045807 | positive regulation of | 1.09E−04 | 3.40E−03 | 8.86 | 2589 | 33 | 62 | 7 |
| | endocytosis | | | | | | | |
| GO:0022613 | ribonucleoprotein complex | 1.13E−04 | 3.52E−03 | 1.89 | 2589 | 42 | 976 | 30 |
| | biogenesis | | | | | | | |
| GO:1903035 | negative regulation of | 1.20E−04 | 3.71E−03 | 4.51 | 2589 | 18 | 319 | 10 |
| | response to wounding | | | | | | | |
| GO:0045089 | positive regulation of | 1.23E−04 | 3.78E−03 | 2.81 | 2589 | 25 | 590 | 16 |
| | innate immune response | | | | | | | |
| GO:1904589 | regulation of protein | 1.29E−04 | 3.97E−03 | 4.19 | 2589 | 22 | 309 | 11 |
| | import | | | | | | | |
| GO:0043632 | modification-dependent | 1.32E−04 | 4.06E−03 | 1.67 | 2589 | 94 | 841 | 51 |
| | macromolecule catabolic | | | | | | | |
| | process | | | | | | | |
| GO:0051336 | regulation of hydrolase | 1.33E−04 | 4.07E−03 | 3.66 | 2589 | 180 | 59 | 15 |
| | activity | | | | | | | |
| GO:0019730 | antimicrobial humoral | 1.36E−04 | 4.14E−03 | 3.45 | 2589 | 18 | 500 | 12 |
| | response | | | | | | | |
| GO:0048856 | anatomical structure | 1.38E−04 | 4.19E−03 | 2.59 | 2589 | 349 | 63 | 22 |
| | development | | | | | | | |
| GO:0051094 | positive regulation of | 1.43E−04 | 4.33E−03 | 1.46 | 2589 | 181 | 862 | 88 |
| | developmental process | | | | | | | |
| GO:0002675 | positive regulation of acute | 1.44E−04 | 4.35E−03 | 31.32 | 2589 | 4 | 62 | 3 |
| | inflammatory response | | | | | | | |
| GO:0031532 | actin cytoskeleton | 1.50E−04 | 4.50E−03 | 5.69 | 2589 | 9 | 354 | 7 |
| | reorganization | | | | | | | |
| GO:0051726 | regulation of cell cycle | 1.52E−04 | 4.56E−03 | 1.61 | 2589 | 132 | 757 | 62 |
| GO:0002684 | positive regulation of | 1.52E−04 | 4.54E−03 | 1.76 | 2589 | 120 | 601 | 49 |
| | immune system process | | | | | | | |
| GO:0050778 | positive regulation of | 1.55E−04 | 4.61E−03 | 1.63 | 2589 | 81 | 983 | 50 |
| | immune response | | | | | | | |
| GO:0052548 | regulation of endopeptidase | 1.61E−04 | 4.77E−03 | 3.38 | 2589 | 73 | 168 | 16 |
| | activity | | | | | | | |
| GO:0042221 | response to chemical | 1.64E−04 | 4.86E−03 | 2.22 | 2589 | 474 | 69 | 28 |
| GO:0002706 | regulation of lymphocyte | 1.67E−04 | 4.92E−03 | 10.23 | 2589 | 23 | 66 | 6 |
| | mediated immunity | | | | | | | |
| GO:0044092 | negative regulation of | 1.73E−04 | 5.07E−03 | 3.82 | 2589 | 161 | 59 | 14 |
| | molecular function | | | | | | | |
| GO:1903047 | mitotic cell cycle process | 1.75E−04 | 5.11E−03 | 1.88 | 2589 | 58 | 806 | 34 |
| GO:0051099 | positive regulation of | 1.78E−04 | 5.18E−03 | 2.17 | 2589 | 49 | 632 | 26 |
| | binding | | | | | | | |
| GO:0043242 | negative regulation of | 1.81E−04 | 5.27E−03 | 2.46 | 2589 | 23 | 779 | 17 |
| | protein complex | | | | | | | |
| | disassembly | | | | | | | |
| GO:0006955 | immune response | 1.85E−04 | 5.37E−03 | 1.77 | 2589 | 103 | 655 | 46 |
| GO:0002708 | positive regulation of | 1.90E−04 | 5.48E−03 | 13.08 | 2589 | 15 | 66 | 5 |
| | lymphocyte mediated | | | | | | | |
| | immunity | | | | | | | |
| GO:0002824 | positive regulation of | 1.90E−04 | 5.46E−03 | 13.08 | 2589 | 15 | 66 | 5 |
| | adaptive immune response | | | | | | | |
| | based on somatic | | | | | | | |
| | recombination of immune | | | | | | | |
| | receptors built from | | | | | | | |
| | immunoglobulin | | | | | | | |
| | superfamily domains | | | | | | | |
| GO:0009967 | positive regulation of | 1.93E−04 | 5.54E−03 | 2.16 | 2589 | 175 | 226 | 33 |
| | signal transduction | | | | | | | |
| GO:0071495 | cellular response to | 2.03E−04 | 5.81E−03 | 2.05 | 2589 | 101 | 425 | 34 |
| | endogenous stimulus | | | | | | | |
| GO:0071353 | cellular response to | 2.08E−04 | 5.94E−03 | 46.23 | 2589 | 12 | 14 | 3 |
| | interleukin-4 | | | | | | | |
| GO:0070670 | response to interleukin-4 | 2.08E−04 | 5.92E−03 | 46.23 | 2589 | 12 | 14 | 3 |
| GO:0071157 | negative regulation of cell | 2.15E−04 | 6.10E−03 | 13.19 | 2589 | 5 | 157 | 4 |
| | cycle arrest | | | | | | | |
| GO:1902531 | regulation of intracellular | 2.15E−04 | 6.08E−03 | 1.97 | 2589 | 201 | 262 | 40 |
| | signal transduction | | | | | | | |
| GO:0014002 | astrocyte development | 2.20E−04 | 6.21E−03 | 20.07 | 2589 | 3 | 129 | 3 |
| GO:0051093 | negative regulation of | 2.24E−04 | 6.28E−03 | 2.02 | 2589 | 109 | 411 | 35 |
| | developmental process | | | | | | | |
| GO:0045944 | positive regulation of | 2.27E−04 | 6.34E−03 | 1.55 | 2589 | 104 | 964 | 60 |
| | transcription by RNA | | | | | | | |
| | polymerase II | | | | | | | |
| GO:0044085 | cellular component | 2.39E−04 | 6.67E−03 | 1.85 | 2589 | 43 | 976 | 30 |
| | biogenesis | | | | | | | |
| GO:0002699 | positive regulation of | 2.40E−04 | 6.67E−03 | 7.85 | 2589 | 35 | 66 | 7 |
| | immune effector process | | | | | | | |
| GO:0032970 | regulation of actin | 2.41E−04 | 6.67E−03 | 1.65 | 2589 | 83 | 908 | 48 |
| | filament-based process | | | | | | | |
| GO:2000144 | positive regulation of | 2.43E−04 | 6.72E−03 | 4.06 | 2589 | 9 | 567 | 8 |
| | DNA-templated | | | | | | | |
| | transcription, initiation | | | | | | | |
| GO:2000142 | regulation of DNA- | 2.43E−04 | 6.70E−03 | 4.06 | 2589 | 9 | 567 | 8 |
| | templated transcription, | | | | | | | |
| | initiation | | | | | | | |
| GO:0042730 | fibrinolysis | 2.46E−04 | 6.75E−03 | 4.8 | 2589 | 8 | 472 | 7 |
| GO:0034660 | ncRNA metabolic process | 2.53E−04 | 6.94E−03 | 1.53 | 2589 | 106 | 992 | 62 |
| GO:0045785 | positive regulation of cell | 2.60E−04 | 7.11E−03 | 3.83 | 2589 | 56 | 157 | 13 |
| | adhesion | | | | | | | |
| GO:0006414 | translational elongation | 2.61E−04 | 7.11E−03 | 8.36 | 2589 | 13 | 143 | 6 |
| GO:0022603 | regulation of anatomical | 2.62E−04 | 7.10E−03 | 1.8 | 2589 | 140 | 472 | 46 |
| | structure morphogenesis | | | | | | | |
| GO:0006810 | transport | 2.71E−04 | 7.33E−03 | 1.57 | 2589 | 551 | 203 | 68 |
| GO:0045087 | innate immune response | 2.76E−04 | 7.44E−03 | 1.92 | 2589 | 70 | 655 | 34 |
| GO:1900182 | positive regulation of | 2.89E−04 | 7.77E−03 | 3.88 | 2589 | 23 | 319 | 11 |
| | protein localization to | | | | | | | |
| | nucleus | | | | | | | |
| GO:0034249 | negative regulation of | 2.90E−04 | 7.78E−03 | 2.7 | 2589 | 40 | 432 | 18 |
| | cellular amide metabolic | | | | | | | |
| | process | | | | | | | |
| GO:0031334 | positive regulation of | 2.94E−04 | 7.87E−03 | 1.94 | 2589 | 52 | 769 | 30 |
| | protein complex assembly | | | | | | | |
| GO:0033036 | macromolecule localization | 3.02E−04 | 8.05E−03 | 1.26 | 2589 | 346 | 994 | 168 |
| GO:0009895 | negative regulation of | 3.05E−04 | 8.12E−03 | 2.11 | 2589 | 72 | 494 | 29 |
| | catabolic process | | | | | | | |
| GO:0050708 | regulation of protein | 3.06E−04 | 8.10E−03 | 2.52 | 2589 | 68 | 333 | 22 |
| | secretion | | | | | | | |
| GO:0010976 | positive regulation of | 3.15E−04 | 8.33E−03 | 7.71 | 2589 | 48 | 49 | 7 |
| | neuron projection | | | | | | | |
| | development | | | | | | | |
| GO:0051187 | cofactor catabolic process | 3.16E−04 | 8.34E−03 | 12.96 | 2589 | 27 | 37 | 5 |
| GO:1905952 | regulation of lipid | 3.20E−04 | 8.41E−03 | 9.28 | 2589 | 27 | 62 | 6 |
| | localization | | | | | | | |
| GO:0051347 | positive regulation of | 3.28E−04 | 8.59E−03 | 2.25 | 2589 | 70 | 428 | 26 |
| | transferase activity | | | | | | | |
| GO:0060284 | regulation of cell | 3.37E−04 | 8.80E−03 | 1.51 | 2589 | 130 | 899 | 68 |
| | development | | | | | | | |
| GO:0090066 | regulation of anatomical | 3.61E−04 | 9.39E−03 | 1.88 | 2589 | 88 | 580 | 37 |
| | structure size | | | | | | | |
| GO:0051346 | negative regulation of | 3.62E−04 | 9.40E−03 | 1.75 | 2589 | 73 | 812 | 40 |
| | hydrolase activity | | | | | | | |
| GO:0042176 | regulation of protein | 3.62E−04 | 9.38E−03 | 1.72 | 2589 | 84 | 769 | 43 |
| | catabolic process | | | | | | | |
| GO:0016525 | negative regulation of | 3.75E−04 | 9.69E−03 | 2.52 | 2589 | 13 | 947 | 12 |
| | angiogenesis | | | | | | | |
| GO:0001934 | positive regulation of | 3.79E−04 | 9.75E−03 | 1.63 | 2589 | 99 | 820 | 51 |
| | protein phosphorylation | | | | | | | |
| GO:1901796 | regulation of signal | 3.83E−04 | 9.83E−03 | 6.14 | 2589 | 14 | 211 | 7 |
| | transduction by p53 class | | | | | | | |
| | mediator | | | | | | | |
| GO:0043066 | negative regulation of | 3.85E−04 | 9.85E−03 | 4.81 | 2589 | 163 | 33 | 10 |
| | apoptotic process | | | | | | | |
| GO:0031936 | negative regulation of | 3.85E−04 | 9.82E−03 | 7.47 | 2589 | 6 | 289 | 5 |
| | chromatin silencing | | | | | | | |
| GO:0043161 | proteasome-mediated | 3.87E−04 | 9.84E−03 | 2.02 | 2589 | 60 | 619 | 29 |
| | ubiquitin-dependent | | | | | | | |
| | protein catabolic process | | | | | | | |
| GO:0010873 | positive regulation of | 3.93E−04 | 9.97E−03 | 11.51 | 2589 | 5 | 180 | 4 |
| | cholesterol esterification | | | | | | | |
| GO:0034370 | triglyceride-rich | 3.93E−04 | 9.95E−03 | 11.51 | 2589 | 5 | 180 | 4 |
| | lipoprotein particle | | | | | | | |
| | remodeling | | | | | | | |
| GO:0002673 | regulation of acute | 3.93E−04 | 9.93E−03 | 16.7 | 2589 | 10 | 62 | 4 |
| | inflammatory response | | | | | | | |
| GO:0050766 | positive regulation of | 3.93E−04 | 9.90E−03 | 16.7 | 2589 | 10 | 62 | 4 |
| | phagocytosis | | | | | | | |
| GO:0032663 | regulation of interleukin-2 | 3.94E−04 | 9.90E−03 | 9.15 | 2589 | 4 | 283 | 4 |
| | production | | | | | | | |
| GO:0030300 | regulation of intestinal | 4.00E−04 | 1.00E−02 | 26.33 | 2589 | 5 | 59 | 3 |
| | cholesterol absorption | | | | | | | |
| GO:1904729 | regulation of intestinal | 4.00E−04 | 9.99E−03 | 26.33 | 2589 | 5 | 59 | 3 |
| | lipid absorption | | | | | | | |
| GO:1904478 | regulation of intestinal | 4.00E−04 | 9.96E−03 | 26.33 | 2589 | 5 | 59 | 3 |
| | absorption | | | | | | | |
| GO:0031401 | positive regulation of | 4.05E−04 | 1.00E−02 | 2.39 | 2589 | 138 | 196 | 25 |
| | protein modification | | | | | | | |
| | process | | | | | | | |
| GO:0030837 | negative regulation of actin | 4.12E−04 | 1.02E−02 | 2.42 | 2589 | 22 | 779 | 16 |
| | filament polymerization | | | | | | | |
| GO:0019731 | antibacterial humoral | 4.14E−04 | 1.02E−02 | 3.27 | 2589 | 8 | 791 | 8 |
| | response | | | | | | | |
| GO:0001819 | positive regulation of | 4.20E−04 | 1.03E−02 | 5.21 | 2589 | 41 | 109 | 9 |
| | cytokine production | | | | | | | |
| GO:0046824 | positive regulation of | 4.21E−04 | 1.03E−02 | 2.36 | 2589 | 21 | 837 | 16 |
| | nucleocytoplasmic | | | | | | | |
| | transport | | | | | | | |
| GO:0042327 | positive regulation of | 4.22E−04 | 1.03E−02 | 1.58 | 2589 | 114 | 820 | 57 |
| | phosphorylation | | | | | | | |
| GO:0051641 | cellular localization | 4.22E−04 | 1.03E−02 | 1.26 | 2589 | 350 | 994 | 169 |
| GO:0051495 | positive regulation of | 4.23E−04 | 1.03E−02 | 2.04 | 2589 | 46 | 717 | 26 |
| | cytoskeleton organization | | | | | | | |
| GO:0050807 | regulation of synapse | 4.25E−04 | 1.03E−02 | 9.06 | 2589 | 35 | 49 | 6 |
| | organization | | | | | | | |
| GO:0045898 | regulation of RNA | 4.28E−04 | 1.04E−02 | 4.66 | 2589 | 6 | 556 | 6 |
| | polymerase II | | | | | | | |
| | transcriptional preinitiation | | | | | | | |
| | complex assembly | | | | | | | |
| GO:0045899 | positive regulation of RNA | 4.28E−04 | 1.03E−02 | 4.66 | 2589 | 6 | 556 | 6 |
| | polymerase II | | | | | | | |
| | transcriptional preinitiation | | | | | | | |
| | complex assembly | | | | | | | |
| GO:1900048 | positive regulation of | 4.30E−04 | 1.04E−02 | 4.42 | 2589 | 8 | 512 | 7 |
| | hemostasis | | | | | | | |
| GO:0030194 | positive regulation of blood | 4.30E−04 | 1.03E−02 | 4.42 | 2589 | 8 | 512 | 7 |
| | coagulation | | | | | | | |
| GO:0050820 | positive regulation of | 4.30E−04 | 1.03E−02 | 4.42 | 2589 | 8 | 512 | 7 |
| | coagulation | | | | | | | |
| GO:1901576 | organic substance | 4.43E−04 | 1.06E−02 | 1.41 | 2589 | 567 | 334 | 103 |
| | biosynthetic process | | | | | | | |
| GO:0043484 | regulation of RNA splicing | 4.52E−04 | 1.08E−02 | 3.31 | 2589 | 46 | 238 | 14 |
| GO:0043280 | positive regulation of | 4.62E−04 | 1.10E−02 | 11.85 | 2589 | 26 | 42 | 5 |
| | cysteine-type | | | | | | | |
| | endopeptidase activity | | | | | | | |
| | involved in apoptotic | | | | | | | |
| | process | | | | | | | |
| GO:0070494 | regulation of thrombin- | 4.66E−04 | 1.10E−02 | 64.73 | 2589 | 2 | 40 | 2 |
| | activated receptor signaling | | | | | | | |
| | pathway | | | | | | | |
| GO:0070495 | negative regulation of | 4.66E−04 | 1.10E−02 | 64.73 | 2589 | 2 | 40 | 2 |
| | thrombin- activated | | | | | | | |
| | receptor signaling pathway | | | | | | | |
| GO:0051621 | regulation of | 4.66E−04 | 1.10E−02 | 64.73 | 2589 | 2 | 40 | 2 |
| | norepinephrine uptake | | | | | | | |
| GO:0043069 | negative regulation of | 4.80E−04 | 1.13E−02 | 4.7 | 2589 | 167 | 33 | 10 |
| | programmed cell death | | | | | | | |
| GO:0051234 | establishment of | 4.88E−04 | 1.15E−02 | 1.54 | 2589 | 571 | 203 | 69 |
| | localization | | | | | | | |
| GO:0032870 | cellular response to | 4.90E−04 | 1.15E−02 | 2.89 | 2589 | 43 | 333 | 16 |
| | hormone stimulus | | | | | | | |
| GO:0043524 | negative regulation of | 4.94E−04 | 1.15E−02 | 18.79 | 2589 | 29 | 19 | 4 |
| | neuron apoptotic process | | | | | | | |
| GO:0034605 | cellular response to heat | 4.95E−04 | 1.15E−02 | 6.23 | 2589 | 17 | 171 | 7 |
| GO:0001817 | regulation of cytokine | 5.00E−04 | 1.16E−02 | 4.73 | 2589 | 73 | 75 | 10 |
| | production | | | | | | | |
| GO:0042744 | hydrogen peroxide | 5.00E−04 | 1.16E−02 | 17.49 | 2589 | 16 | 37 | 4 |
| | catabolic process | | | | | | | |
| GO:0015893 | drug transport | 5.07E−04 | 1.17E−02 | 36.99 | 2589 | 21 | 10 | 3 |
| GO:0051053 | negative regulation of | 5.09E−04 | 1.17E−02 | 3.99 | 2589 | 21 | 309 | 10 |
| | DNA metabolic process | | | | | | | |
| GO:0070488 | neutrophil aggregation | 5.14E−04 | 1.18E−02 | 61.64 | 2589 | 2 | 42 | 2 |
| GO:1901700 | response to oxygen- | 5.25E−04 | 1.20E−02 | 2.87 | 2589 | 203 | 80 | 18 |
| | containing compound | | | | | | | |
| GO:0034116 | positive regulation of | 5.33E−04 | 1.22E−02 | 10.57 | 2589 | 5 | 196 | 4 |
| | heterotypic cell-cell | | | | | | | |
| | adhesion | | | | | | | |
| GO:0002714 | positive regulation of B | 5.42E−04 | 1.24E−02 | 23.54 | 2589 | 5 | 66 | 3 |
| | cell mediated immunity | | | | | | | |
| GO:0002891 | positive regulation of | 5.42E−04 | 1.23E−02 | 23.54 | 2589 | 5 | 66 | 3 |
| | immunoglobulin mediated | | | | | | | |
| | immune response | | | | | | | |
| GO:0010499 | proteasomal ubiquitin- | 5.43E−04 | 1.23E−02 | 3.28 | 2589 | 17 | 510 | 11 |
| | independent protein | | | | | | | |
| | catabolic process | | | | | | | |
| GO:0044249 | cellular biosynthetic | 5.50E−04 | 1.25E−02 | 1.41 | 2589 | 536 | 338 | 99 |
| | process | | | | | | | |
| GO:0071384 | cellular response to | 5.57E−04 | 1.26E−02 | 5.83 | 2589 | 8 | 333 | 6 |
| | corticosteroid stimulus | | | | | | | |
| GO:0050817 | coagulation | 5.63E−04 | 1.27E−02 | 4.16 | 2589 | 16 | 350 | 9 |
| GO:0007596 | blood coagulation | 5.63E−04 | 1.27E−02 | 4.16 | 2589 | 16 | 350 | 9 |
| GO:0048821 | erythrocyte development | 5.66E−04 | 1.27E−02 | 129.45 | 2589 | 10 | 4 | 2 |
| GO:0009057 | macromolecule catabolic | 5.67E−04 | 1.27E−02 | 1.46 | 2589 | 158 | 852 | 76 |
| | process | | | | | | | |
| GO:0032956 | regulation of actin | 5.74E−04 | 1.28E−02 | 1.66 | 2589 | 72 | 908 | 42 |
| | cytoskeleton organization | | | | | | | |
| GO:1902806 | regulation of cell cycle | 5.81E−04 | 1.29E−02 | 2.73 | 2589 | 18 | 686 | 13 |
| | Gl/S phase transition | | | | | | | |
| GO:0045861 | negative regulation of | 5.82E−04 | 1.29E−02 | 2.01 | 2589 | 66 | 566 | 29 |
| | proteolysis | | | | | | | |
| GO:0060341 | regulation of cellular | 5.88E−04 | 1.30E−02 | 1.66 | 2589 | 183 | 469 | 55 |
| | localization | | | | | | | |
| GO:0045666 | positive regulation of | 6.03E−04 | 1.33E−02 | 6.98 | 2589 | 53 | 49 | 7 |
| | neuron differentiation | | | | | | | |
| GO:0043244 | regulation of protein | 6.04E−04 | 1.33E−02 | 2.11 | 2589 | 32 | 805 | 21 |
| | complex disassembly | | | | | | | |
| GO:0000910 | cytokinesis | 6.04E−04 | 1.33E−02 | 2.82 | 2589 | 16 | 688 | 12 |
| GO:0061640 | cytoskeleton-dependent | 6.04E−04 | 1.33E−02 | 2.82 | 2589 | 16 | 688 | 12 |
| | cytokinesis | | | | | | | |
| GO:0007051 | spindle organization | 6.20E−04 | 1.36E−02 | 3.98 | 2589 | 23 | 283 | 10 |
| GO:0032535 | regulation of cellular | 6.23E−04 | 1.36E−02 | 1.83 | 2589 | 67 | 717 | 34 |
| | component size | | | | | | | |
| GO:0045787 | positive regulation of cell | 6.35E−04 | 1.38E−02 | 2.82 | 2589 | 57 | 274 | 17 |
| | cycle | | | | | | | |
| GO:0006310 | DNA recombination | 6.41E−04 | 1.39E−02 | 2.1 | 2589 | 23 | 964 | 18 |
| GO:0001932 | regulation of protein | 6.51E−04 | 1.41E−02 | 1.46 | 2589 | 164 | 820 | 76 |
| | phosphorylation | | | | | | | |
| GO:0043086 | negative regulation of | 6.52E−04 | 1.41E−02 | 1.55 | 2589 | 119 | 812 | 58 |
| | catalytic activity | | | | | | | |
| GO:0045744 | negative regulation of G | 6.63E−04 | 1.43E−02 | 14.07 | 2589 | 3 | 184 | 3 |
| | protein-coupled receptor | | | | | | | |
| | signaling pathway | | | | | | | |
| GO:1904029 | regulation of cyclin- | 6.74E−04 | 1.45E−02 | 105.67 | 2589 | 7 | 7 | 2 |
| | dependent protein kinase | | | | | | | |
| | activity | | | | | | | |
| GO:0010720 | positive regulation of cell | 6.79E−04 | 1.46E−02 | 1.61 | 2589 | 84 | 899 | 47 |
| | development | | | | | | | |
| GO:0002793 | positive regulation of | 6.80E−04 | 1.46E−02 | 4.01 | 2589 | 48 | 148 | 11 |
| | peptide secretion | | | | | | | |
| GO:0060968 | regulation of gene | 6.88E−04 | 1.47E−02 | 3.35 | 2589 | 19 | 447 | 11 |
| | silencing | | | | | | | |
| GO:1903900 | regulation of viral life | 7.04E−04 | 1.50E−02 | 3 | 2589 | 33 | 366 | 14 |
| | cycle | | | | | | | |
| GO:0034470 | ncRNA processing | 7.05E−04 | 1.50E−02 | 2.74 | 2589 | 73 | 233 | 18 |
| GO:0030163 | protein catabolic process | 7.14E−04 | 1.51E−02 | 1.78 | 2589 | 91 | 622 | 39 |
| GO:1904591 | positive regulation of | 7.21E−04 | 1.52E−02 | 4.19 | 2589 | 18 | 309 | 9 |
| | protein import | | | | | | | |
| GO:0048585 | negative regulation of | 7.24E−04 | 1.53E−02 | 1.62 | 2589 | 196 | 472 | 58 |
| | response to stimulus | | | | | | | |
| GO:0000226 | microtubule cytoskeleton | 7.68E−04 | 1.62E−02 | 3.93 | 2589 | 45 | 161 | 11 |
| | organization | | | | | | | |
| GO:0001907 | killing by symbiont of host | 7.72E−04 | 1.62E−02 | 1,294.50 | 2589 | 1 | 2 | 1 |
| | cells | | | | | | | |
| GO:0052331 | hemolysis in other | 7.72E−04 | 1.62E−02 | 1,294.50 | 2589 | 1 | 2 | 1 |
| | organism involved in | | | | | | | |
| | symbiotic interaction | | | | | | | |
| GO:0001897 | cytolysis by symbiont of | 7.72E−04 | 1.62E−02 | 1,294.50 | 2589 | 1 | 2 | 1 |
| | host cells | | | | | | | |
| GO:0045938 | positive regulation of | 7.72E−04 | 1.61E−02 | 1,294.50 | 2589 | 1 | 2 | 1 |
| | circadian sleep/wake cycle, | | | | | | | |
| | sleep | | | | | | | |
| GO:0045187 | regulation of circadian | 7.72E−04 | 1.61E−02 | 1,294.50 | 2589 | 1 | 2 | 1 |
| | sleep/wake cycle, sleep | | | | | | | |
| GO:0045188 | regulation of circadian | 7.72E−04 | 1.60E−02 | 1,294.50 | 2589 | 1 | 2 | 1 |
| | sleep/wake cycle, non- | | | | | | | |
| | REM sleep | | | | | | | |
| GO:0042749 | regulation of circadian | 7.72E−04 | 1.60E−02 | 1,294.50 | 2589 | 1 | 2 | 1 |
| | sleep/wake cycle | | | | | | | |
| GO:0042753 | positive regulation of | 7.72E−04 | 1.60E−02 | 1,294.50 | 2589 | 1 | 2 | 1 |
| | circadian rhythm | | | | | | | |
| GO:0046010 | positive regulation of | 7.72E−04 | 1.59E−02 | 1,294.50 | 2589 | 1 | 2 | 1 |
| | circadian sleep/wake cycle, | | | | | | | |
| | non-REM sleep | | | | | | | |
| GO:0044179 | hemolysis in other | 7.72E−04 | 1.59E−02 | 1,294.50 | 2589 | 1 | 2 | 1 |
| | organism | | | | | | | |
| GO:0044004 | disruption by symbiont of | 7.72E−04 | 1.59E−02 | 1,294.50 | 2589 | 1 | 2 | 1 |
| | host cell | | | | | | | |
| GO:0051659 | maintenance of | 7.72E−04 | 1.58E−02 | 1,294.50 | 2589 | 1 | 2 | 1 |
| | mitochondrion location | | | | | | | |
| GO:0019836 | hemolysis by symbiont of | 7.72E−04 | 1.58E−02 | 1,294.50 | 2589 | 1 | 2 | 1 |
| | host erythrocytes | | | | | | | |
| GO:0044770 | cell cycle phase transition | 7.74E−04 | 1.58E−02 | 2.72 | 2589 | 13 | 805 | 11 |
| GO:0050792 | regulation of viral process | 7.76E−04 | 1.58E−02 | 2.67 | 2589 | 45 | 366 | 17 |
| GO:0050658 | RNA transport | 7.76E−04 | 1.57E−02 | 4.91 | 2589 | 50 | 95 | 9 |
| GO:0050657 | nucleic acid transport | 7.76E−04 | 1.57E−02 | 4.91 | 2589 | 50 | 95 | 9 |
| GO:0051236 | establishment of RNA | 7.76E−04 | 1.57E−02 | 4.91 | 2589 | 50 | 95 | 9 |
| | localization | | | | | | | |
| GO:0042026 | protein refolding | 7.92E−04 | 1.60E−02 | 6.79 | 2589 | 13 | 176 | 6 |
| GO:0048869 | cellular developmental | 8.01E−04 | 1.61E−02 | 2.81 | 2589 | 289 | 51 | 16 |
| | process | | | | | | | |
| GO:0010498 | proteasomal protein | 8.13E−04 | 1.63E−02 | 1.93 | 2589 | 65 | 619 | 30 |
| | catabolic process | | | | | | | |
| GO:2001056 | positive regulation of | 8.15E−04 | 1.63E−02 | 10.63 | 2589 | 29 | 42 | 5 |
| | cysteine-type | | | | | | | |
| | endopeptidase activity | | | | | | | |
| GO:0060260 | regulation of transcription | 8.61E−04 | 1.72E−02 | 4 | 2589 | 8 | 567 | 7 |
| | initiation from RNA | | | | | | | |
| | polymerase II promoter | | | | | | | |
| GO:0060261 | positive regulation of | 8.61E−04 | 1.72E−02 | 4 | 2589 | 8 | 567 | 7 |
| | transcription initiation from | | | | | | | |
| | RNA polymerase II | | | | | | | |
| | promoter | | | | | | | |
| GO:0061478 | response to platelet | 8.78E−04 | 1.75E−02 | 52.3 | 2589 | 3 | 33 | 2 |
| | aggregation inhibitor | | | | | | | |
| GO:0045815 | positive regulation of gene | 8.81E−04 | 1.75E−02 | 3.42 | 2589 | 9 | 673 | 8 |
| | expression, epigenetic | | | | | | | |
| GO:0050715 | positive regulation of | 8.82E−04 | 1.75E−02 | 9.14 | 2589 | 13 | 109 | 5 |
| | cytokine secretion | | | | | | | |
| GO:0043691 | reverse cholesterol | 9.22E−04 | 1.82E−02 | 21.94 | 2589 | 6 | 59 | 3 |
| | transport | | | | | | | |
| GO:0010872 | regulation of cholesterol | 9.22E−04 | 1.82E−02 | 21.94 | 2589 | 6 | 59 | 3 |
| | esterification | | | | | | | |
| GO:0034380 | high-density lipoprotein | 9.22E−04 | 1.81E−02 | 21.94 | 2589 | 6 | 59 | 3 |
| | particle assembly | | | | | | | |
| GO:0033700 | phospholipid efflux | 9.22E−04 | 1.81E−02 | 21.94 | 2589 | 6 | 59 | 3 |
| GO:0030490 | maturation of SSU-rRNA | 9.24E−04 | 1.81E−02 | 8.04 | 2589 | 9 | 179 | 5 |
| GO:0021782 | glial cell development | 9.25E−04 | 1.81E−02 | 11.47 | 2589 | 7 | 129 | 4 |
| GO:0010466 | negative regulation of | 9.32E−04 | 1.82E−02 | 2.19 | 2589 | 46 | 566 | 22 |
| | peptidase activity | | | | | | | |
| GO:0051016 | barbed-end actin filament | 9.36E−04 | 1.82E−02 | 2.99 | 2589 | 10 | 779 | 9 |
| | capping | | | | | | | |
| GO:0032368 | regulation of lipid transport | 9.69E−04 | 1.88E−02 | 9.97 | 2589 | 22 | 59 | 5 |
| GO:0071897 | DNA biosynthetic process | 9.73E−04 | 1.89E−02 | 4.29 | 2589 | 9 | 469 | 7 |

Analysis of the function shows multiple significant functional differences between the HepG2 than in the normal liver, these including nucleic acid binding, protein binding oxygen binding expressed higher in the tumor (Table 17, Table 18).

Analysis by component showed large different in cytosolic part, protein-containing complex, ribonucleoprotein complex extracted from the HepG2 vs the normal liver (Table 19, Table 20).

TABLE 17
Gene ontology by a process of the differently expressed proteins in the
HepG2 the normal liver extracted with electroporation mapped with Gorilla

| | | | FDR | Enrichment |
|---|---|---|---|---|
| GO term | Description | P-value* | q-value** | (N, B, n, b)*** |
| GO:0043170 | macromolecule metabolic | 2.47E−23 | 2.22E−19 | 1.36 (2589, 919, 992, 478) |
| | process | | | |
| GO:0044260 | cellular macromolecule | 1.98E−21 | 8.93E−18 | 1.44 (2589, 648, 992, 358) |
| | metabolic process | | | |
| GO:0090304 | nucleic acid metabolic | 5.93E−20 | 1.78E−16 | 1.64 (2589, 334, 992, 210) |
| | process | | | |
| GO:0050789 | regulation of biological | 7.73E−20 | 1.74E−16 | 1.25 (2589, 1301, 968, 607) |
| | process | | | |
| GO:0051171 | regulation of nitrogen | 1.08E−19 | 1.94E−16 | 1.57 (2589, 695, 639, 269) |
| | compound metabolic | | | |
| | process | | | |
| GO:0050794 | regulation of cellular | 1.55E−19 | 2.33E−16 | 1.27 (2589, 1202, 970, 570) |
| | process | | | |
| GO:0060255 | regulation of | 1.86E−19 | 2.4E−16 | 1.52 (2589, 718, 689, 291) |
| | macromolecule metabolic | | | |
| | process | | | |
| GO:0080090 | regulation of primary | 8.83E−19 | 9.95E−16 | 1.54 (2589, 731, 639, 277) |
| | metabolic process | | | |
| GO:0006412 | translation | 1.07E−16 | 1.07E−13 | 2.83 (2589, 161, 398, 70) |
| GO:0048519 | negative regulation of | 1.57E−16 | 1.41E−13 | 1.45 (2589, 720, 759, 306) |
| | biological process | | | |
| GO:0034645 | cellular macromolecule | 2.77E−16 | 2.27E−13 | 2.50 (2589, 231, 381, 85) |
| | biosynthetic process | | | |
| GO:0009059 | macromolecule | 4.38E−16 | 3.29E−13 | 2.45 (2589, 241, 381, 87) |
| | biosynthetic process | | | |
| GO:0048523 | negative regulation of | 4.78E−16 | 3.31E−13 | 1.47 (2589, 655, 759, 283) |
| | cellular process | | | |
| GO:0043043 | peptide biosynthetic | 5.81E−16 | 3.74E−13 | 2.76 (2589, 165, 398, 70) |
| | process | | | |
| GO:0010468 | regulation of gene | 7.68E−16 | 4.61E−13 | 1.53 (2589, 480, 846, 240) |
| | expression | | | |

*‘P-value’ is the enrichment p-value computed according to the mHG or HG model. This p-value is not corrected for multiple testing of 731 GO terms.
**‘FDR q-value’ is the correction of the above p-value for multiple testing using the Benjamini and Hochberg (1995) method.
Namely, for the i^{th} term (ranked according to p-value) the FDR q-value is (p-value * a number of GO terms)/i.
***Enrichment (N, B, n, b) is defined as follows:
N - is the total number of genes
B - is the total number of genes associated with a specific GO term
n - is the number of genes in the top of the user's input list or in the target set when appropriate
b - is the number of genes in the intersection
Enrichment = (b/n)/(B/N)

TABLE 18

| GO Term | Description | P-value | FDR q-value | Enrichment | N | B | n | b |
|---|---|---|---|---|---|---|---|---|
| GO:0003676 | nucleic acid binding | 5.63E−39 | 1.46E−35 | 1.73 | 2589 | 461 | 991 | 306 |
| GO:0003723 | RNA binding | 5.41E−31 | 7.02E−28 | 1.79 | 2589 | 342 | 981 | 232 |
| GO:0005515 | protein binding | 2.13E−28 | 1.85E−25 | 1.26 | 2589 | 1435 | 999 | 696 |
| GO:0003735 | structural constituent of | 5.96E−24 | 3.87E−21 | 4.18 | 2589 | 95 | 372 | 57 |
| | ribosome | | | | | | | |
| GO:0005198 | structural molecule | 1.29E−21 | 6.73E−19 | 2.89 | 2589 | 191 | 398 | 85 |
| | activity | | | | | | | |
| GO:0005488 | binding | 6.16E−19 | 2.67E−16 | 1.12 | 2589 | 2046 | 999 | 884 |
| GO:0003729 | mRNA binding | 2.28E−15 | 8.47E−13 | 2.53 | 2589 | 98 | 679 | 65 |
| GO:0003677 | DNA binding | 1.74E−14 | 5.66E−12 | 1.81 | 2589 | 163 | 991 | 113 |
| GO:0044877 | protein-containing | 1.19E−13 | 3.44E−11 | 1.6 | 2589 | 324 | 890 | 178 |
| | complex binding | | | | | | | |
| GO:0019899 | enzyme binding | 1.34E−12 | 3.49E−10 | 1.82 | 2589 | 463 | 375 | 122 |
| GO:0019843 | rRNA binding | 3.13E−12 | 7.39E−10 | 4.73 | 2589 | 33 | 398 | 24 |
| GO:1901363 | heterocyclic compound | 9.98E−12 | 2.16E−09 | 1.23 | 2589 | 973 | 992 | 460 |
| | binding | | | | | | | |
| GO:0097159 | organic cyclic | 3.02E−11 | 6.04E−09 | 1.22 | 2589 | 995 | 992 | 467 |
| | compound binding | | | | | | | |
| GO:0008092 | cytoskeletal protein | 1.98E−10 | 3.68E−08 | 1.76 | 2589 | 198 | 756 | 102 |
| | binding | | | | | | | |
| GO:0043565 | sequence-specific DNA | 2.18E−10 | 3.77E−08 | 2.18 | 2589 | 73 | 845 | 52 |
| | binding | | | | | | | |
| GO:0019825 | oxygen binding | 1.06E−09 | 1.73E−07 | 235.36 | 2589 | 11 | 4 | 4 |
| GO:0003779 | actin binding | 1.69E−09 | 2.58E−07 | 2.26 | 2589 | 99 | 614 | 53 |
| GO:0051082 | unfolded protein binding | 1.75E−09 | 2.52E−07 | 5.7 | 2589 | 49 | 176 | 19 |
| GO:0003690 | double-stranded DNA | 1.81E−09 | 2.48E−07 | 1.97 | 2589 | 73 | 991 | 55 |
| | binding | | | | | | | |
| GO:0008134 | transcription factor | 1.85E−09 | 2.41E−07 | 1.82 | 2589 | 108 | 964 | 73 |
| | binding | | | | | | | |
| GO:0031721 | hemoglobin alpha | 1.02E−08 | 1.27E−06 | 485.44 | 2589 | 4 | 4 | 3 |
| | binding | | | | | | | |
| GO:0061134 | peptidase regulator | 1.54E−08 | 1.82E−06 | 2.99 | 2589 | 47 | 535 | 29 |
| | activity | | | | | | | |
| GO:0043021 | ribonucleoprotein | 1.96E−08 | 2.21E−06 | 2.09 | 2589 | 59 | 925 | 44 |
| | complex binding | | | | | | | |
| GO:0031720 | haptoglobin binding | 3.98E−08 | 4.31E−06 | 388.35 | 2589 | 5 | 4 | 3 |
| GO:0030492 | hemoglobin binding | 8.18E−08 | 8.50E−06 | 323.62 | 2589 | 6 | 4 | 3 |
| GO:0003730 | mRNA 3′-UTR binding | 8.68E−08 | 8.67E−06 | 3.21 | 2589 | 27 | 627 | 21 |
| GO:1990837 | sequence-specific | 9.75E−08 | 9.38E−06 | 2.2 | 2589 | 53 | 845 | 38 |
| | double-stranded DNA | | | | | | | |
| | binding | | | | | | | |
| GO:0005102 | signaling receptor | 2.09E−07 | 1.94E−05 | 3.39 | 2589 | 242 | 79 | 25 |
| | binding | | | | | | | |
| GO:0051015 | actin filament binding | 2.82E−07 | 2.53E−05 | 2.23 | 2589 | 65 | 696 | 39 |
| GO:0061135 | endopeptidase regulator | 2.97E−07 | 2.58E−05 | 2.43 | 2589 | 39 | 791 | 29 |
| | activity | | | | | | | |
| GO:0004857 | enzyme inhibitor activity | 3.91E−07 | 3.28E−05 | 2.02 | 2589 | 71 | 812 | 45 |
| GO:0030234 | enzyme regulator | 5.12E−07 | 4.16E−05 | 1.64 | 2589 | 176 | 791 | 88 |
| | activity | | | | | | | |
| GO:0050542 | icosanoid binding | 8.25E−07 | 6.49E−05 | 49.31 | 2589 | 5 | 42 | 4 |
| GO:0001067 | regulatory region nucleic | 1.65E−06 | 1.26E−04 | 2.08 | 2589 | 55 | 838 | 37 |
| | acid binding | | | | | | | |
| GO:0031072 | heat shock protein | 2.11E−06 | 1.56E−04 | 2.01 | 2589 | 46 | 981 | 35 |
| | binding | | | | | | | |
| GO:0030414 | peptidase inhibitor | 2.62E−06 | 1.89E−04 | 3.03 | 2589 | 35 | 513 | 21 |
| | activity | | | | | | | |
| GO:0008135 | translation factor | 2.69E−06 | 1.89E−04 | 2.2 | 2589 | 41 | 863 | 30 |
| | activity, RNA binding | | | | | | | |
| GO:0044212 | transcription regulatory | 3.51E−06 | 2.40E−04 | 2.08 | 2589 | 52 | 838 | 35 |
| | region DNA binding | | | | | | | |
| GO:0098772 | molecular function | 4.14E−06 | 2.76E−04 | 3.62 | 2589 | 219 | 62 | 19 |
| | regulator | | | | | | | |
| GO:0048027 | mRNA 5′-UTR binding | 4.36E−06 | 2.83E−04 | 4.78 | 2589 | 14 | 426 | 11 |
| GO:0042826 | histone deacetylase | 6.41E−06 | 4.06E−04 | 4.62 | 2589 | 14 | 440 | 11 |
| | binding | | | | | | | |
| GO:0008289 | lipid binding | 7.01E−06 | 4.33E−04 | 4.34 | 2589 | 154 | 62 | 16 |
| GO:0004866 | endopeptidase inhibitor | 7.77E−06 | 4.69E−04 | 2.97 | 2589 | 34 | 513 | 20 |
| | activity | | | | | | | |
| GO:0000976 | transcription regulatory | 1.64E−05 | 9.69E−04 | 1.98 | 2589 | 41 | 991 | 31 |
| | region sequence-specific | | | | | | | |
| | DNA binding | | | | | | | |
| GO:0003743 | translation initiation | 1.67E−05 | 9.66E−04 | 2.3 | 2589 | 30 | 863 | 23 |
| | factor activity | | | | | | | |
| GO:0019901 | protein kinase binding | 1.69E−05 | 9.53E−04 | 1.61 | 2589 | 133 | 844 | 70 |
| GO:0019904 | protein domain specific | 1.97E−05 | 1.09E−03 | 1.5 | 2589 | 150 | 991 | 86 |
| | binding | | | | | | | |
| GO:0031625 | ubiquitin protein ligase | 2.14E−05 | 1.16E−03 | 2.23 | 2589 | 77 | 497 | 33 |
| | binding | | | | | | | |
| GO:0042802 | identical protein binding | 2.15E−05 | 1.14E−03 | 1.36 | 2589 | 463 | 642 | 156 |
| GO:0070180 | large ribosomal subunit | 2.64E−05 | 1.37E−03 | 8.6 | 2589 | 7 | 258 | 6 |
| | rRNA binding | | | | | | | |
| GO:0003725 | double-stranded RNA | 2.75E−05 | 1.40E−03 | 3.32 | 2589 | 29 | 430 | 16 |
| | binding | | | | | | | |
| GO:0005344 | oxygen carrier activity | 3.11E−05 | 1.56E−03 | 323.62 | 2589 | 4 | 4 | 2 |
| GO:0005504 | fatty acid binding | 3.30E−05 | 1.62E−03 | 13.58 | 2589 | 22 | 52 | 6 |
| GO:0050544 | arachidonic acid binding | 4.09E−05 | 1.97E−03 | 46.23 | 2589 | 4 | 42 | 3 |
| GO:0004601 | peroxidase activity | 5.06E−05 | 2.39E−03 | 71.92 | 2589 | 27 | 4 | 3 |
| GO:0016684 | oxidoreductase activity, | 5.67E−05 | 2.63E−03 | 69.35 | 2589 | 28 | 4 | 3 |
| | acting on peroxide as | | | | | | | |
| | acceptor | | | | | | | |
| GO:0044389 | ubiquitin-like protein | 7.85E−05 | 3.58E−03 | 2.12 | 2589 | 81 | 497 | 33 |
| | ligase binding | | | | | | | |
| GO:0140110 | transcription regulator | 8.02E−05 | 3.59E−03 | 1.64 | 2589 | 87 | 964 | 53 |
| | activity | | | | | | | |
| GO:0019900 | kinase binding | 8.60E−05 | 3.79E−03 | 1.53 | 2589 | 150 | 844 | 75 |
| GO:0016209 | antioxidant activity | 9.01E−05 | 3.90E−03 | 9.38 | 2589 | 46 | 42 | 7 |
| GO:0001158 | enhancer sequence- | 9.86E−05 | 4.20E−03 | 18.17 | 2589 | 6 | 95 | 4 |
| | specific DNA binding | | | | | | | |
| GO:0035326 | enhancer binding | 9.86E−05 | 4.13E−03 | 18.17 | 2589 | 6 | 95 | 4 |
| GO:0036094 | small molecule binding | 1.03E−04 | 4.25E−03 | 2.85 | 2589 | 633 | 23 | 16 |
| GO:0008144 | drug binding | 1.19E−04 | 4.84E−03 | 6.06 | 2589 | 374 | 8 | 7 |
| GO:0008035 | high-density lipoprotein | 1.21E−04 | 4.84E−03 | 32.91 | 2589 | 4 | 59 | 3 |
| | particle binding | | | | | | | |
| GO:0033293 | monocarboxylic acid | 1.24E−04 | 4.89E−03 | 11.06 | 2589 | 27 | 52 | 6 |
| | binding | | | | | | | |
| GO:0050543 | icosatetraenoic acid | 1.35E−04 | 5.22E−03 | 36.99 | 2589 | 5 | 42 | 3 |
| | binding | | | | | | | |
| GO:0000977 | RNA polymerase II | 1.76E−04 | 6.74E−03 | 2.22 | 2589 | 37 | 726 | 23 |
| | regulatory region | | | | | | | |
| | sequence-specific DNA | | | | | | | |
| | binding | | | | | | | |
| GO:0001012 | RNA polymerase II | 1.76E−04 | 6.64E−03 | 2.22 | 2589 | 37 | 726 | 23 |
| | regulatory region DNA | | | | | | | |
| | binding | | | | | | | |
| GO:0032564 | dATP binding | 1.86E−04 | 6.89E−03 | 11.06 | 2589 | 4 | 234 | 4 |
| GO:0017025 | TBP-class protein | 1.93E−04 | 7.05E−03 | 4.14 | 2589 | 9 | 556 | 8 |
| | binding | | | | | | | |
| GO:0005200 | structural constituent of | 1.93E−04 | 6.96E−03 | 2.28 | 2589 | 23 | 887 | 18 |
| | cytoskeleton | | | | | | | |
| GO:0030957 | Tat protein binding | 2.23E−04 | 7.94E−03 | 101.53 | 2589 | 3 | 17 | 2 |
| GO:0005253 | anion channel activity | 2.56E−04 | 8.99E−03 | 6.72 | 2589 | 5 | 385 | 5 |
| GO:0005092 | GDP-dissociation | 2.77E−04 | 9.61E−03 | 6.59 | 2589 | 5 | 393 | 5 |
| | inhibitor activity | | | | | | | |
| GO:0002020 | protease binding | 3.43E−04 | 1.17E−02 | 9.44 | 2589 | 35 | 47 | 6 |
| GO:0036402 | proteasome-activating | 3.45E−04 | 1.17E−02 | 4.8 | 2589 | 6 | 539 | 6 |
| | ATPase activity | | | | | | | |
| GO:0043177 | organic acid binding | 3.57E−04 | 1.19E−02 | 5.53 | 2589 | 81 | 52 | 9 |
| GO:0008097 | 5S rRNA binding | 3.59E−04 | 1.18E−02 | 7.6 | 2589 | 6 | 284 | 5 |
| GO:0031722 | hemoglobin beta binding | 3.86E−04 | 1.25E−02 | 2,589.00 | 2589 | 1 | 1 | 1 |
| GO:0060228 | phosphatidylcholine- | 3.93E−04 | 1.26E−02 | 11.51 | 2589 | 5 | 180 | 4 |
| | sterol O-acyltransferase | | | | | | | |
| | activator activity | | | | | | | |
| GO:0017091 | AU-rich element binding | 4.27E−04 | 1.35E−02 | 5.48 | 2589 | 7 | 405 | 6 |
| GO:0031490 | chromatin DNA binding | 4.32E−04 | 1.35E−02 | 3.21 | 2589 | 20 | 484 | 12 |
| GO:0036002 | pre-mRNA binding | 5.10E−04 | 1.58E−02 | 2.45 | 2589 | 13 | 975 | 12 |
| GO:0003682 | chromatin binding | 5.35E−04 | 1.64E−02 | 1.58 | 2589 | 81 | 991 | 49 |
| GO:0004298 | threonine-type | 5.43E−04 | 1.64E−02 | 3.28 | 2589 | 17 | 510 | 11 |
| | endopeptidase activity | | | | | | | |
| GO:0070003 | threonine-type peptidase | 5.43E−04 | 1.62E−02 | 3.28 | 2589 | 17 | 510 | 11 |
| | activity | | | | | | | |
| GO:0071813 | lipoprotein particle | 5.54E−04 | 1.64E−02 | 107.88 | 2589 | 6 | 8 | 2 |
| | binding | | | | | | | |
| GO:0071814 | protein-lipid complex | 5.54E−04 | 1.62E−02 | 107.88 | 2589 | 6 | 8 | 2 |
| | binding | | | | | | | |
| GO:1990715 | mRNA CDS binding | 5.65E−04 | 1.63E−02 | 14.88 | 2589 | 3 | 174 | 3 |
| GO:0017111 | nucleoside- | 5.85E−04 | 1.67E−02 | 1.4 | 2589 | 176 | 946 | 90 |
| | triphosphatase activity | | | | | | | |
| GO:0008201 | heparin binding | 6.64E−04 | 1.88E−02 | 2.19 | 2589 | 25 | 851 | 18 |
| GO:0002039 | p53 binding | 6.68E−04 | 1.87E−02 | 15.14 | 2589 | 12 | 57 | 4 |
| GO:0001091 | RNA polymerase II | 7.28E−04 | 2.01E−02 | 4.26 | 2589 | 6 | 608 | 6 |
| | basal transcription factor | | | | | | | |
| | binding | | | | | | | |
| GO:0000987 | proximal promoter | 7.60E−04 | 2.08E−02 | 2.11 | 2589 | 21 | 991 | 17 |
| | sequence-specific DNA | | | | | | | |
| | binding | | | | | | | |
| GO:0000978 | RNA polymerase II | 7.60E−04 | 2.06E−02 | 2.11 | 2589 | 21 | 991 | 17 |
| | proximal promoter | | | | | | | |
| | sequence-specific DNA | | | | | | | |
| | binding | | | | | | | |
| GO:0003684 | damaged DNA binding | 7.83E−04 | 2.10E−02 | 3.65 | 2589 | 13 | 491 | 9 |
| GO:0050786 | RAGE receptor binding | 7.87E−04 | 2.09E−02 | 20.71 | 2589 | 5 | 75 | 3 |
| GO:0000981 | DNA-binding | 8.15E−04 | 2.14E−02 | 2.04 | 2589 | 25 | 964 | 19 |
| | transcription factor | | | | | | | |
| | activity, RNA | | | | | | | |
| | polymerase Il-specific | | | | | | | |
| GO:0055106 | ubiquitin-protein | 8.45E−04 | 2.20E−02 | 13.08 | 2589 | 3 | 198 | 3 |
| | transferase regulator | | | | | | | |
| | activity | | | | | | | |
| GO:0005543 | phospholipid binding | 8.68E−04 | 2.23E−02 | 4.05 | 2589 | 80 | 88 | 11 |
| GO:0035257 | nuclear hormone | 9.74E−04 | 2.48E−02 | 1.99 | 2589 | 27 | 962 | 20 |
| | receptor binding | | | | | | | |

TABLE 19
Gene ontology by function of the differently expressed proteins in the HepG2
the normal liver extracted with electroporation mapped with Gorilla

| | | | FDR | Enrichment |
|---|---|---|---|---|
| GO term | Description | P-value* | q-value** | (N, B, n, b)*** |
| GO:0003676 | nucleic acid binding | 5.63E−39 | 1.46E−35 | 1.73 (2589, 461, 991, 306) |
| GO:0003723 | RNA binding | 5.41E−31 | 7.02E−28 | 1.79 (2589, 342, 981, 232) |
| GO:0005515 | protein binding | 2.13E−28 | 1.85E−25 | 1.26 (2589, 1435, 999, 696) |
| GO:0003735 | structural constituent | 5.96E−24 | 3.87E−21 | 4.18 (2589, 95, 372, 57) |
| | of ribosome | | | |
| GO:0005198 | structural molecule | 1.29E−21 | 6.73E−19 | 2.89 (2589, 191, 398, 85) |
| | activity | | | |
| GO:0005488 | binding | 6.16E−19 | 2.67E−16 | 1.12 (2589, 2046, 999, 884) |
| GO:0003729 | mRNA binding | 2.28E−15 | 8.47E−13 | 2.53 (2589, 98, 679, 65) |
| GO:0003677 | DNA binding | 1.74E−14 | 5.66E−12 | 1.81 (2589, 163, 991, 113) |
| GO:0044877 | protein-containing | 1.19E−13 | 3.44E−11 | 1.60 (2589, 324, 890, 178) |
| | complex binding | | | |
| GO:0019899 | enzyme binding | 1.34E−12 | 3.49E−10 | 1.82 (2589, 463, 375, 122) |
| GO:0019843 | rRNA binding | 3.13E−12 | 7.39E−10 | 4.73 (2589, 33, 398, 24) |
| GO:1901363 | heterocyclic | 9.98E−12 | 2.16E−9 | 1.23 (2589, 973, 992, 460) |
| | compound binding | | | |
| GO:0097159 | organic cyclic | 3.02E−11 | 6.04E−9 | 1.22 (2589, 995, 992, 467) |
| | compound binding | | | |
| GO:0008092 | cytoskeletal protein | 1.98E−10 | 3.68E−8 | 1.76 (2589, 198, 756, 102) |
| | binding | | | |
| GO:0043565 | sequence-specific | 2.18E−10 | 3.77E−8 | 2.18 (2589, 73, 845, 52) |
| | DNA binding | | | |

*‘P-value’ is the enrichment p-value computed according to the mHG or HG model. This p-value is not corrected for multiple testing of 731 GO terms.
**‘FDR q-value’ is the correction of the above p-value for multiple testing using the Benjamini and Hochberg (1995) method.
Namely, for the i^{th} term (ranked according to p-value) the FDR q-value is (p-value * a number of GO terms)/i.
***Enrichment (N, B, n, b) is defined as follows:
N - is the total number of genes
B - is the total number of genes associated with a specific GO term
n - is the number of genes in the top of the user's input list or in the target set when appropriate
b - is the number of genes in the intersection
Enrichment = (b/n)/(B/N)

TABLE 20

| GO Term | Description | P-value | FDR q-value | Enrichment | N | B | n | b |
|---|---|---|---|---|---|---|---|---|
| GO:0044445 | cytosolic part | 3.36E−41 | 4.19E−38 | 4.74 | 2589 | 119 | 372 | 81 |
| GO:0032991 | protein-containing | 5.55E−40 | 3.46E−37 | 1.4 | 2589 | 1107 | 994 | 593 |
| | complex | | | | | | | |
| GO:1990904 | ribonucleoprotein | 1.08E−31 | 4.49E−29 | 2.16 | 2589 | 323 | 662 | 178 |
| | complex | | | | | | | |
| GO:0005634 | nucleus | 2.43E−31 | 7.59E−29 | 1.41 | 2589 | 922 | 991 | 498 |
| GO:0043232 | intracellular non- | 1.04E−25 | 2.58E−23 | 1.51 | 2589 | 597 | 991 | 345 |
| | membrane-bounded | | | | | | | |
| | organelle | | | | | | | |
| GO:0043228 | non-membrane-bounded | 1.34E−25 | 2.79E−23 | 1.51 | 2589 | 602 | 991 | 347 |
| | organelle | | | | | | | |
| GO:0044391 | ribosomal subunit | 4.82E−24 | 8.59E−22 | 3.92 | 2589 | 110 | 372 | 62 |
| GO:0044428 | nuclear part | 2.50E−23 | 3.90E−21 | 1.43 | 2589 | 720 | 992 | 394 |
| GO:0005840 | ribosome | 1.87E−22 | 2.59E−20 | 3.83 | 2589 | 109 | 372 | 60 |
| GO:0005737 | cytoplasm | 9.86E−22 | 1.23E−19 | 1.25 | 2589 | 1339 | 973 | 629 |
| GO:0022625 | cytosolic large | 3.54E−21 | 4.01E−19 | 4.58 | 2589 | 43 | 500 | 38 |
| | ribosomal subunit | | | | | | | |
| GO:0045202 | synapse | 1.00E−17 | 1.04E−15 | 2.54 | 2589 | 192 | 456 | 86 |
| GO:0022627 | cytosolic small | 4.33E−17 | 4.16E−15 | 5.72 | 2589 | 35 | 362 | 28 |
| | ribosomal subunit | | | | | | | |
| GO:0005681 | spliceosomal complex | 1.39E−13 | 1.24E−11 | 2.12 | 2589 | 80 | 975 | 64 |
| GO:0097458 | neuron part | 1.42E−13 | 1.18E−11 | 1.6 | 2589 | 321 | 840 | 167 |
| GO:0015934 | large ribosomal subunit | 4.06E−13 | 3.17E−11 | 4.01 | 2589 | 65 | 338 | 34 |
| GO:0015935 | small ribosomal subunit | 6.03E−13 | 4.42E−11 | 4.41 | 2589 | 47 | 362 | 29 |
| GO:0043209 | myelin sheath | 2.06E−12 | 1.43E−10 | 4.57 | 2589 | 109 | 161 | 31 |
| GO:0044456 | synapse part | 2.19E−12 | 1.44E−10 | 1.85 | 2589 | 181 | 789 | 102 |
| GO:0044421 | extracellular region part | 2.79E−12 | 1.74E−10 | 3.78 | 2589 | 292 | 82 | 35 |
| GO:0005856 | cytoskeleton | 4.97E−12 | 2.95E−10 | 1.63 | 2589 | 241 | 896 | 136 |
| GO:0044430 | cytoskeletal part | 1.36E−11 | 7.69E−10 | 1.68 | 2589 | 258 | 757 | 127 |
| GO:0071013 | catalytic step 2 | 1.88E−11 | 1.02E−09 | 2.36 | 2589 | 45 | 974 | 40 |
| | spliceosome | | | | | | | |
| GO:0030863 | cortical cytoskeleton | 3.15E−11 | 1.64E−09 | 3.41 | 2589 | 38 | 579 | 29 |
| GO:0000502 | proteasome complex | 1.40E−10 | 6.98E−09 | 2.28 | 2589 | 48 | 969 | 41 |
| GO:0005832 | chaperonin-containing | 2.27E−10 | 1.09E−08 | 18.76 | 2589 | 8 | 138 | 8 |
| | T-complex | | | | | | | |
| GO:1905369 | endopeptidase complex | 5.38E−10 | 2.49E−08 | 2.24 | 2589 | 49 | 969 | 41 |
| GO:0101031 | chaperone complex | 6.71E−10 | 2.99E−08 | 12.14 | 2589 | 17 | 138 | 11 |
| GO:0005615 | extracellular space | 7.83E−10 | 3.37E−08 | 3.89 | 2589 | 227 | 82 | 28 |
| GO:0042788 | polysomal ribosome | 8.60E−10 | 3.57E−08 | 5.45 | 2589 | 23 | 351 | 17 |
| GO:0044427 | chromosomal part | 1.11E−09 | 4.48E−08 | 1.74 | 2589 | 126 | 991 | 84 |
| GO:0002199 | zona pellucida receptor | 2.42E−09 | 9.45E−08 | 16.68 | 2589 | 9 | 138 | 8 |
| | complex | | | | | | | |
| GO:0044448 | cell cortex part | 4.28E−09 | 1.62E−07 | 2.47 | 2589 | 59 | 693 | 39 |
| GO:0044422 | organelle part | 4.42E−09 | 1.62E−07 | 1.14 | 2589 | 1468 | 992 | 640 |
| GO:0005654 | nucleoplasm | 5.98E−09 | 2.13E−07 | 1.38 | 2589 | 387 | 991 | 204 |
| GO:0005844 | polysome | 9.73E−09 | 3.37E−07 | 5.34 | 2589 | 24 | 323 | 16 |
| GO:1905368 | peptidase complex | 3.17E−08 | 1.07E−06 | 2.05 | 2589 | 56 | 969 | 43 |
| GO:0005833 | hemoglobin complex | 3.98E−08 | 1.31E−06 | 388.35 | 2589 | 5 | 4 | 3 |
| GO:0044446 | intracellular organelle | 5.46E−08 | 1.75E−06 | 1.13 | 2589 | 1444 | 992 | 626 |
| | part | | | | | | | |
| GO:0014069 | postsynaptic density | 6.37E−08 | 1.99E−06 | 3.22 | 2589 | 69 | 326 | 28 |
| GO:0033267 | axon part | 6.57E−08 | 2.00E−06 | 4.82 | 2589 | 68 | 150 | 19 |
| GO:0005576 | extracellular region | 7.44E−08 | 2.21E−06 | 4.17 | 2589 | 182 | 75 | 22 |
| GO:0031838 | haptoglobin-hemoglobin | 8.18E−08 | 2.37E−06 | 323.62 | 2589 | 6 | 4 | 3 |
| | complex | | | | | | | |
| GO:0042995 | cell projection | 8.64E−08 | 2.45E−06 | 1.4 | 2589 | 306 | 982 | 163 |
| GO:0099572 | postsynaptic | 9.15E−08 | 2.54E−06 | 3.18 | 2589 | 70 | 326 | 28 |
| | specialization | | | | | | | |
| GO:0120025 | plasma membrane | 1.71E−07 | 4.63E−06 | 1.42 | 2589 | 273 | 981 | 147 |
| | bounded cell projection | | | | | | | |
| GO:0035770 | ribonucleoprotein | 2.96E−07 | 7.86E−06 | 1.85 | 2589 | 72 | 991 | 51 |
| | granule | | | | | | | |
| GO:0120038 | plasma membrane | 4.26E−07 | 1.11E−05 | 1.57 | 2589 | 203 | 829 | 102 |
| | bounded cell projection | | | | | | | |
| | part | | | | | | | |
| GO:0044463 | cell projection part | 4.26E−07 | 1.08E−05 | 1.57 | 2589 | 203 | 829 | 102 |
| GO:0005829 | cytosol | 4.68E−07 | 1.17E−05 | 1.21 | 2589 | 823 | 969 | 372 |
| GO:0044451 | nucleoplasm part | 5.87E−07 | 1.43E−05 | 1.55 | 2589 | 157 | 999 | 94 |
| GO:0016604 | nuclear body | 7.26E−07 | 1.74E−05 | 1.63 | 2589 | 119 | 999 | 75 |
| GO:0036464 | cytoplasmic | 7.95E−07 | 1.87E−05 | 1.86 | 2589 | 66 | 991 | 47 |
| | ribonucleoprotein | | | | | | | |
| | granule | | | | | | | |
| GO:0005730 | nucleolus | 1.09E−06 | 2.52E−05 | 1.57 | 2589 | 145 | 991 | 87 |
| GO:0015629 | actin cytoskeleton | 1.41E−06 | 3.20E−05 | 2.22 | 2589 | 53 | 748 | 34 |
| GO:0005684 | U2-type spliceosomal | 1.47E−06 | 3.27E−05 | 2.19 | 2589 | 34 | 974 | 28 |
| | complex | | | | | | | |
| GO:0044297 | cell body | 1.80E−06 | 3.94E−05 | 3.31 | 2589 | 125 | 150 | 24 |
| GO:0048471 | perinuclear region of | 3.00E−06 | 6.44E−05 | 1.56 | 2589 | 144 | 968 | 84 |
| | cytoplasm | | | | | | | |
| GO:0030427 | site of polarized growth | 3.72E−06 | 7.87E−05 | 2.37 | 2589 | 33 | 828 | 25 |
| GO:0030426 | growth cone | 3.72E−06 | 7.74E−05 | 2.37 | 2589 | 33 | 828 | 25 |
| GO:0044454 | nuclear chromosome | 4.29E−06 | 8.77E−05 | 1.77 | 2589 | 74 | 991 | 50 |
| | part | | | | | | | |
| GO:0032993 | protein-DNA complex | 4.43E−06 | 8.92E−05 | 2.3 | 2589 | 25 | 991 | 22 |
| GO:0099513 | polymeric cytoskeletal | 5.35E−06 | 1.06E−04 | 1.65 | 2589 | 122 | 890 | 69 |
| | fiber | | | | | | | |
| GO:0099081 | supramolecular polymer | 5.41E−06 | 1.05E−04 | 1.62 | 2589 | 131 | 890 | 73 |
| GO:0099080 | supramolecular complex | 5.41E−06 | 1.04E−04 | 1.62 | 2589 | 131 | 890 | 73 |
| GO:0099512 | supramolecular fiber | 5.41E−06 | 1.02E−04 | 1.62 | 2589 | 131 | 890 | 73 |
| GO:0098978 | glutamatergic synapse | 5.57E−06 | 1.04E−04 | 1.71 | 2589 | 88 | 961 | 56 |
| GO:0043005 | neuron projection | 6.36E−06 | 1.17E−04 | 1.45 | 2589 | 200 | 981 | 110 |
| GO:0097525 | spliceosomal snRNP | 1.03E−05 | 1.87E−04 | 2.24 | 2589 | 30 | 925 | 24 |
| | complex | | | | | | | |
| GO:0019773 | proteasome core | 1.09E−05 | 1.93E−04 | 6.28 | 2589 | 7 | 412 | 7 |
| | complex, alpha-subunit | | | | | | | |
| | complex | | | | | | | |
| GO:1990124 | messenger | 1.18E−05 | 2.07E−04 | 20.55 | 2589 | 4 | 126 | 4 |
| | ribonucleoprotein | | | | | | | |
| | complex | | | | | | | |
| GO:1902494 | catalytic complex | 1.22E−05 | 2.12E−04 | 1.41 | 2589 | 304 | 780 | 129 |
| GO:0009986 | cell surface | 1.71E−05 | 2.92E−04 | 2.71 | 2589 | 87 | 286 | 26 |
| GO:0044449 | contractile fiber part | 1.85E−05 | 3.11E−04 | 3.12 | 2589 | 41 | 384 | 19 |
| GO:0030532 | small nuclear | 3.01E−05 | 5.00E−04 | 2.28 | 2589 | 31 | 844 | 23 |
| | ribonucleoprotein | | | | | | | |
| | complex | | | | | | | |
| GO:0022624 | proteasome accessory | 3.07E−05 | 5.03E−04 | 3.04 | 2589 | 17 | 702 | 14 |
| | complex | | | | | | | |
| GO:0120114 | Sm-like protein family | 3.30E−05 | 5.34E−04 | 2.23 | 2589 | 33 | 844 | 24 |
| | complex | | | | | | | |
| GO:0016363 | nuclear matrix | 3.49E−05 | 5.58E−04 | 2.86 | 2589 | 29 | 562 | 18 |
| GO:0008540 | proteasome regulatory | 3.91E−05 | 6.17E−04 | 3.39 | 2589 | 12 | 701 | 11 |
| | particle, base | | | | | | | |
| | subcomplex | | | | | | | |
| GO:0043230 | extracellular organelle | 4.75E−05 | 7.41E−04 | 4.78 | 2589 | 24 | 248 | 11 |
| GO:1903561 | extracellular vesicle | 4.75E−05 | 7.32E−04 | 4.78 | 2589 | 24 | 248 | 11 |
| GO:0070062 | extracellular exosome | 4.75E−05 | 7.23E−04 | 4.78 | 2589 | 24 | 248 | 11 |
| GO:0022626 | cytosolic ribosome | 8.30E−05 | 1.25E−03 | 8.35 | 2589 | 5 | 310 | 5 |
| GO:0044306 | neuron projection | 8.72E−05 | 1.29E−03 | 10.67 | 2589 | 16 | 91 | 6 |
| | terminus | | | | | | | |
| GO:0032432 | actin filament bundle | 1.42E−04 | 2.08E−03 | 2.71 | 2589 | 33 | 521 | 18 |
| GO:0015630 | microtubule | 1.42E−04 | 2.07E−03 | 2.03 | 2589 | 32 | 955 | 24 |
| | cytoskeleton | | | | | | | |
| GO:0031012 | extracellular matrix | 1.49E−04 | 2.14E−03 | 2.38 | 2589 | 80 | 353 | 26 |
| GO:0005874 | microtubule | 1.53E−04 | 2.17E−03 | 3.61 | 2589 | 78 | 138 | 15 |
| GO:0005852 | eukaryotic translation | 1.63E−04 | 2.28E−03 | 2.97 | 2589 | 12 | 799 | 11 |
| | initiation factor 3 | | | | | | | |
| | complex | | | | | | | |
| GO:0062023 | collagen-containing | 1.74E−04 | 2.40E−03 | 2.41 | 2589 | 76 | 353 | 25 |
| | extracellular matrix | | | | | | | |
| GO:0000785 | chromatin | 1.76E−04 | 2.42E−03 | 1.69 | 2589 | 65 | 989 | 42 |
| GO:0000786 | nucleosome | 1.88E−04 | 2.54E−03 | 2.93 | 2589 | 12 | 811 | 11 |
| GO:0044815 | DNA packaging | 1.88E−04 | 2.52E−03 | 2.93 | 2589 | 12 | 811 | 11 |
| | complex | | | | | | | |
| GO:0005667 | transcription factor | 1.95E−04 | 2.58E−03 | 2.32 | 2589 | 30 | 744 | 20 |
| | complex | | | | | | | |
| GO:0000791 | euchromatin | 2.30E−04 | 3.02E−03 | 4.44 | 2589 | 10 | 466 | 8 |
| GO:0005719 | nuclear euchromatin | 2.30E−04 | 2.99E−03 | 4.44 | 2589 | 10 | 466 | 8 |
| GO:0016607 | nuclear speck | 2.37E−04 | 3.05E−03 | 1.65 | 2589 | 72 | 980 | 45 |
| GO:0005685 | U1 snRNP | 2.41E−04 | 3.07E−03 | 3.1 | 2589 | 9 | 835 | 9 |
| GO:0000790 | nuclear chromatin | 3.11E−04 | 3.92E−03 | 3.09 | 2589 | 36 | 349 | 15 |
| GO:0098794 | postsynapse | 3.73E−04 | 4.65E−03 | 1.75 | 2589 | 58 | 916 | 36 |
| GO:0071011 | precatalytic spliceosome | 3.82E−04 | 4.72E−03 | 2.33 | 2589 | 18 | 925 | 15 |
| GO:0071005 | U2-type precatalytic | 3.82E−04 | 4.67E−03 | 2.33 | 2589 | 18 | 925 | 15 |
| | spliceosome | | | | | | | |
| GO:0099568 | cytoplasmic region | 4.30E−04 | 5.20E−03 | 2.01 | 2589 | 48 | 725 | 27 |
| GO:0044798 | nuclear transcription | 4.35E−04 | 5.22E−03 | 3.49 | 2589 | 14 | 530 | 10 |
| | factor complex | | | | | | | |
| GO:0090575 | RNA polymerase II | 4.35E−04 | 5.17E−03 | 3.49 | 2589 | 14 | 530 | 10 |
| | transcription factor | | | | | | | |
| | complex | | | | | | | |
| GO:0098793 | presynapse | 5.04E−04 | 5.93E−03 | 2.45 | 2589 | 38 | 529 | 19 |
| GO:0005577 | fibrinogen complex | 5.30E−04 | 6.18E−03 | 10.57 | 2589 | 5 | 196 | 4 |
| GO:0072562 | blood microparticle | 5.30E−04 | 6.12E−03 | 10.57 | 2589 | 5 | 196 | 4 |
| GO:0005839 | proteasome core | 5.43E−04 | 6.21E−03 | 3.28 | 2589 | 17 | 510 | 11 |
| | complex | | | | | | | |
| GO:0070937 | CRD-mediated mRNA | 5.78E−04 | 6.55E−03 | 6.94 | 2589 | 6 | 311 | 5 |
| | stability complex | | | | | | | |
| GO:0043679 | axon terminus | 6.29E−04 | 7.06E−03 | 16.06 | 2589 | 15 | 43 | 4 |
| GO:0034708 | methyltransferase | 6.86E−04 | 7.64E−03 | 2.86 | 2589 | 17 | 638 | 12 |
| | complex | | | | | | | |
| GO:0034719 | SMN-Sm protein | 7.70E−04 | 8.49E−03 | 4.2 | 2589 | 6 | 616 | 6 |
| | complex | | | | | | | |
| GO:0097526 | spliceosomal tri-snRNP | 8.81E−04 | 9.64E−03 | 2.3 | 2589 | 17 | 925 | 14 |
| | complex | | | | | | | |
| GO:0046540 | U4/U6 × U5 tri-snRNP | 8.81E−04 | 9.55E−03 | 2.3 | 2589 | 17 | 925 | 14 |
| | complex | | | | | | | |
| GO:0005689 | U12-type spliceosomal | 8.84E−04 | 9.50E−03 | 3.08 | 2589 | 13 | 646 | 10 |
| | complex | | | | | | | |

### In-Vivo Electroporation Biopsy

The study disclosed herein provides electroporation-biopsy (e-biopsy) procedure protocols to obtain molecular profiles of proteins obtained through this procedure in comparison with currently used lysis buffer extraction. Particularly, it is shown that proteomic profiles obtained by e-biopsy from 4T1 mice tumor in-vivo are tissue specific, show tumor heterogeneity and that they align with molecular information related to these samples extracted using standard lysis buffers from excised tissues.

### Molecular Harvesting In-Vivo

FIG. 11A illustrates the procedure for molecular harvesting in-vivo using electroporation for cell permeabilization: first, an electroporation-electrode-needle is inserted in different locations in the tumor or other tissues; second, once the needle is in place, specific series of high voltage short pulses (PEF-pulses) are applied to permeabilize the cell membrane of nearby cells; third, vacuum is applied on the same needle, through which the PEF pulses are delivered, to suck the tissue liquid (extract) through the needle and into, e.g., a syringe. Next the tissue extract is discharged to an external buffer and is subjected to standard molecular analysis protocols, including purification, separation, identification and quantification. The procedure can be repeated in multiple positions in the same area or other areas multiple times. Moreover, after a single electroporation treatment, liquid (tissue extract) can be harvested in several locations that are electro-permeabilized simultaneously. As seen in FIGS. 11B-11D, 4T1 tumor was sampled six times: two times in the center (C), two times in the periphery (P) and two times in the middle (M) between the center and the periphery. Additional sampling was done in the normal breast at the same animal. All animals survived the procedure and abnormal responses were not observed.

Replicability of In-Vivo e-Biopsy

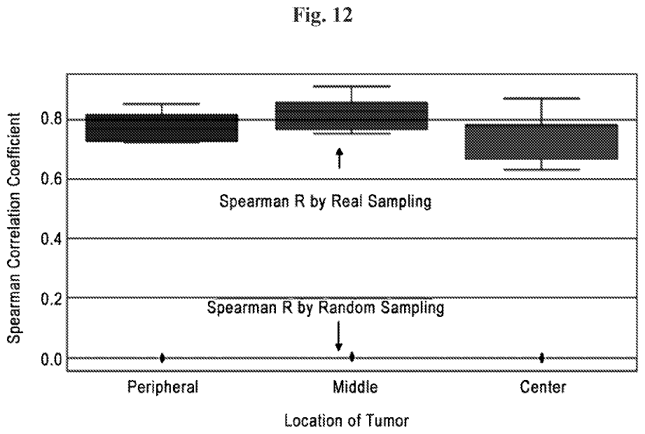

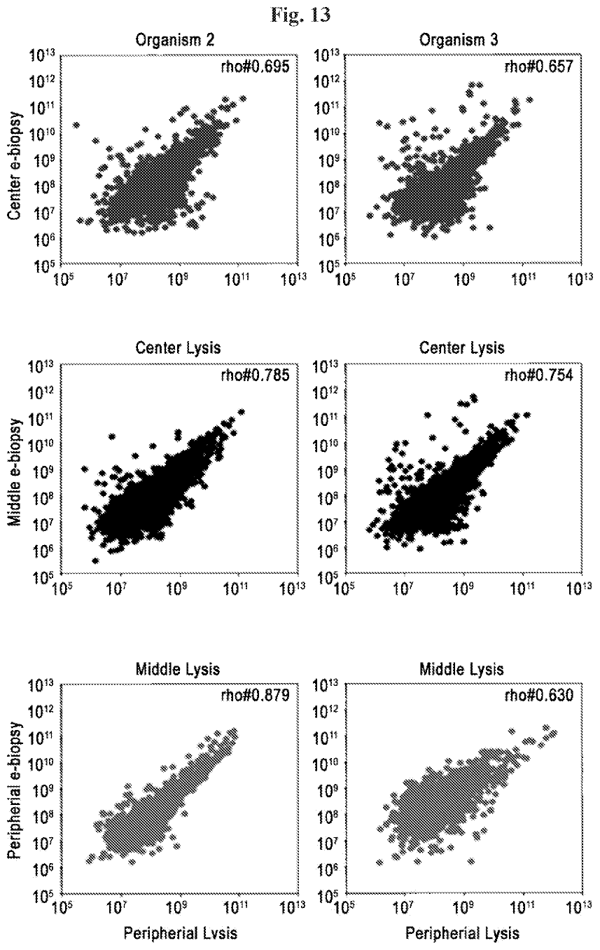

To study the replicability of molecular extraction in-vivo with e-biopsy, liquids (tissue extract) was harvested from the C, M and P positions twice in five 4T1 tumors in-vivo in five mice. In total, 4782 proteins were quantified for each sample using unlabeled proteomics by LC/MS-MS. It was found that the expression level of proteins quantified in the duplicate in close locations had a very strong (Spearman R in 0.63-0.85 range), non-random correlation among themselves (FIG. 12).

In-Vivo e-Biopsy of Proteins Shows a Faithful Molecular Profiling as Compared to Lysis Buffer Extraction of Excised Tissue

Next, to prove that in-vivo harvested by e-biopsy proteins can show truthful molecular map of the tumor, they were compared to proteins extracted from a similar location in excised tumors with standard lysis buffer.

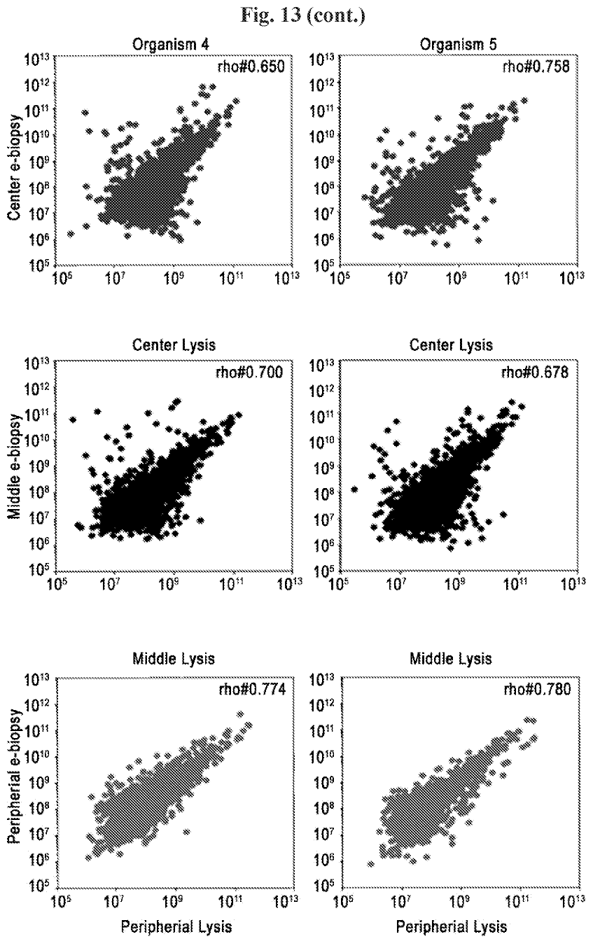

The correlation between proteins extracted with e-biopsy in-vivo with those extracted with a standard lysis buffer from excised tumors showed Spearman R values in range of 0.630 to 0.879 for all three locations in all five animals (FIG. 13). This finding suggests that in-vivo proteins harvesting with e-biopsy in 4T1 tumors is a reliable method that shows the true proteins expression levels at various locations in the tumor.

Proteins Profile Harvested In-Vivo by e-Biopsy Allow for Distinguishing 4T1 Tumor from Normal Breast Tissue in Mice.

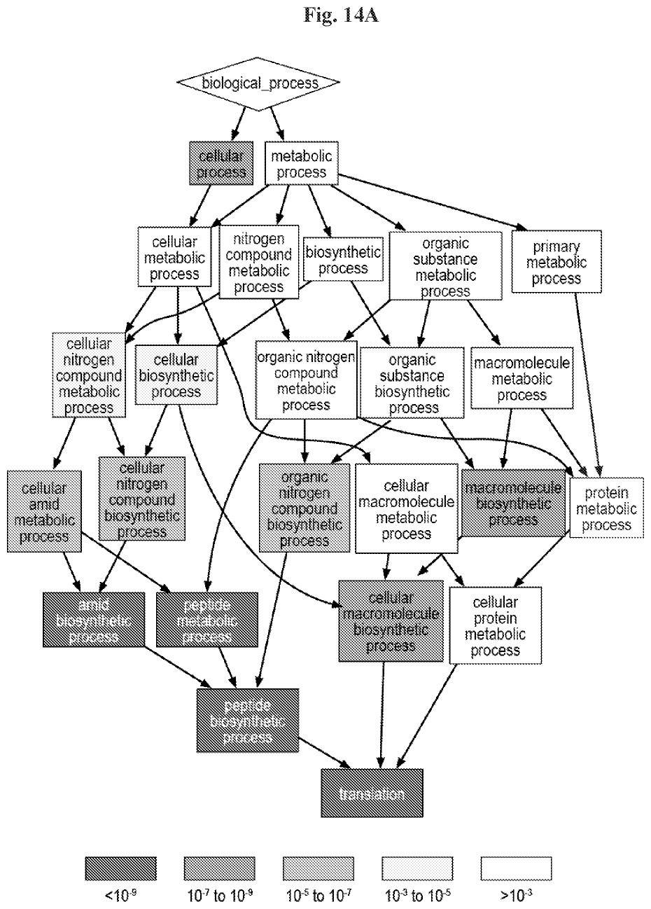

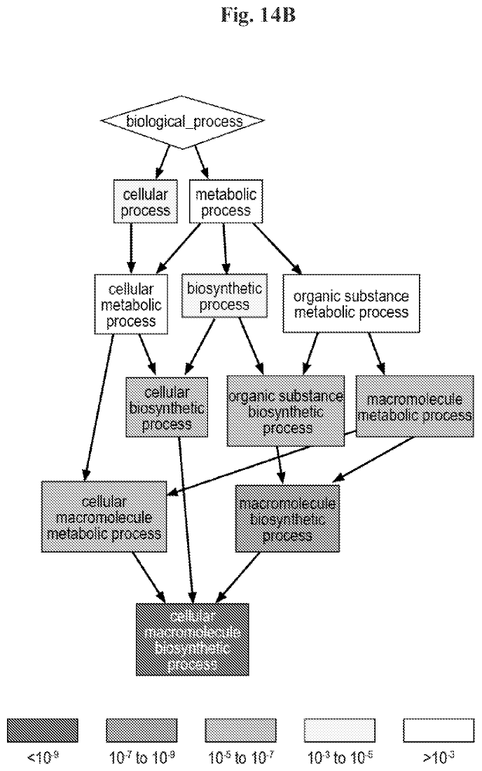

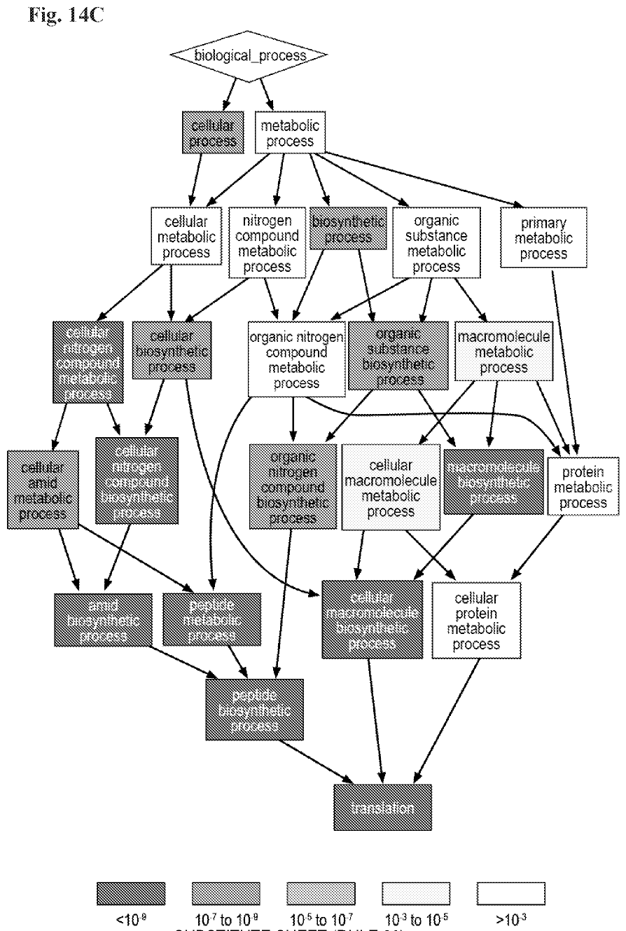

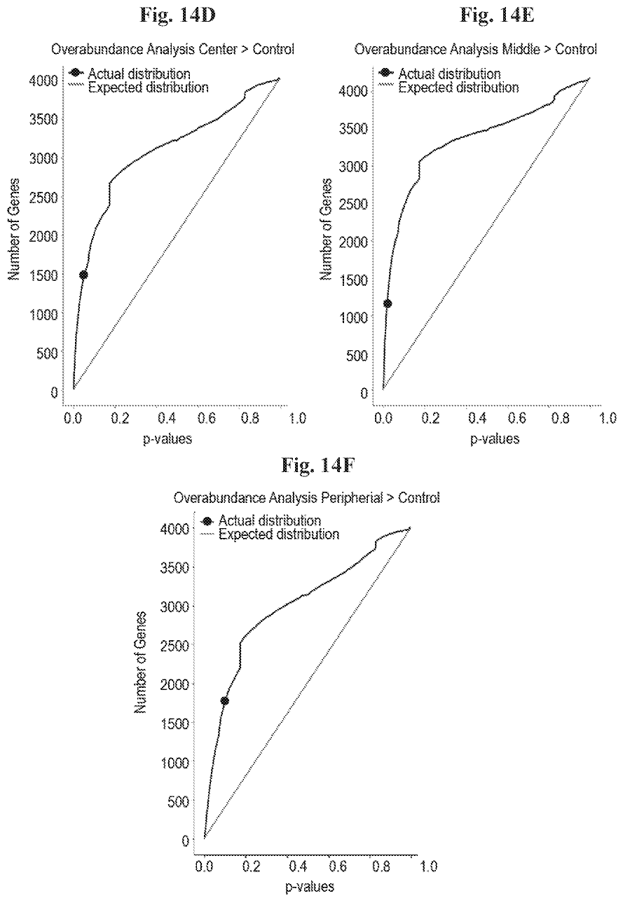

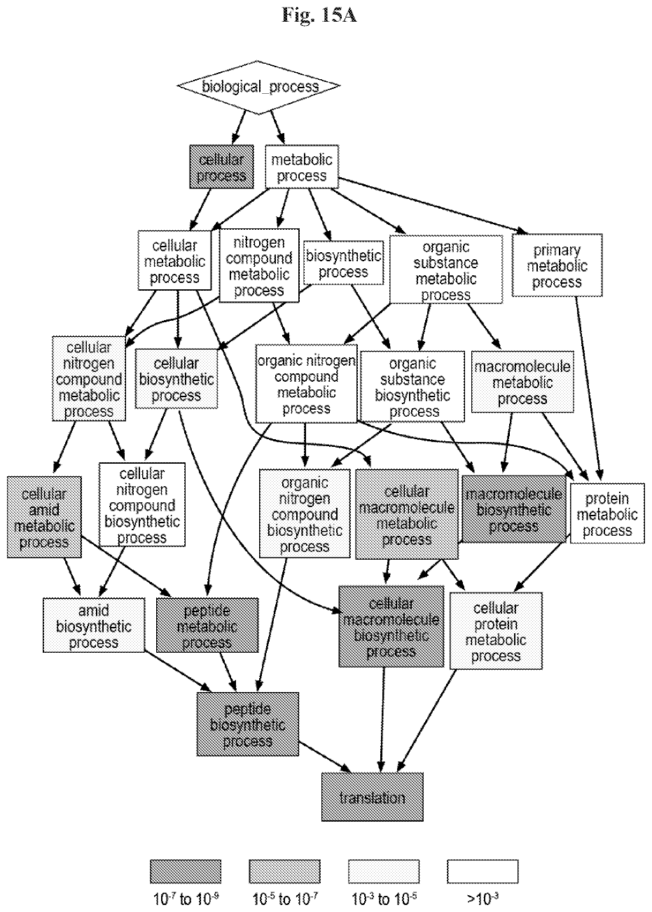

Proteins extracted with e-biopsy from 4T1 tumor and normal mice breast show differential expression levels that are tissue specific. Differential expression analysis was done on three pairs of extracts: 4T1 tumor center (c) vs. Normal breast (NB); 4T1 tumor periphery (P) vs. Normal breast (NB); and 4T1 tumor middle (M) vs. Normal breast (NB). Gene ontology analysis of 4782 extracted proteins showed significant differential expression between proteins expressed in the NB and all three locations in the tumor (FIG. 14).

Specifically: (A) analysis of gene ontology terms by process revealed that translation (FIG. 14A), (p-value: 1.45E−13) was more active in center of the tumor than normal breast; (B) analysis of function categories revealed difference in structural constituent of ribosome (p-value: 3.46E−11) and RNA binding in center of tumor and normal breast (p-value: 6.81E−12); and (C) analysis by component showed differences in cytoplasm (p-value: 5.58E−21) and cytosolic parts (p-value: 3.89E−14).

Gene ontology for middle zone of the tumor was compared with the healthy breast: (A) analysis by process revealed cellular macromolecule biosynthetic process (FIG. 14B) as the most active for the middle region of the tumor (p-value: 2.53E−10); and (B) analysis by function revealed m-RNA binding capacity more active in the M location of the tumor than in normal breast (p-value: 8.52E−10). The component analysis suggested differences in cytosolic large ribosomal subunit (p-value: 2.99E−13) nucleus (p-value: 1.3E−13) and ribosome (p-value: 7.54E−10). Gene ontology analysis for tumor peripheral and control breast revealed translation (FIG. 14C) as active process (p-value: 2.49E−16), followed by m-RNA binding as the most active function (p-value: 1.99E−11) and with component difference in nucleus (p-value: 3.9E−13), cytosolic small ribosomal subunit (p-value: 6.29E−17) and cytosolic large ribosomal subunit (p-value: 1.62E−20). These finding are significant (overabundance plot of differential expression is shown in FIGS. 14D-14F).

Combined, the above data shows that in-vivo e-biopsy of proteins can differentiate 4T1 tumors from normal breast tissue in mice.

In-Vivo e-Biopsy Allows for Dissecting 4T1 Intratumor Proteome Heterogeneity

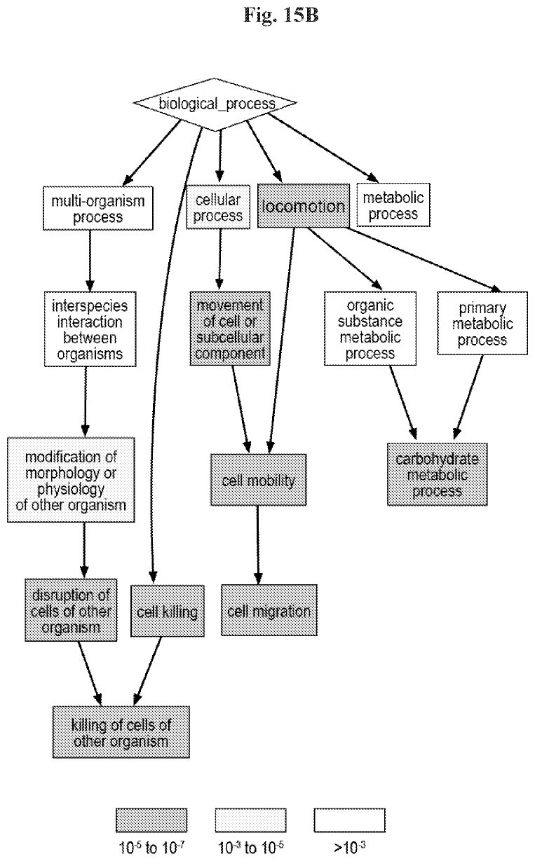

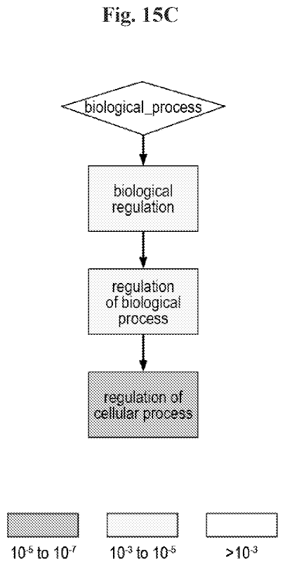

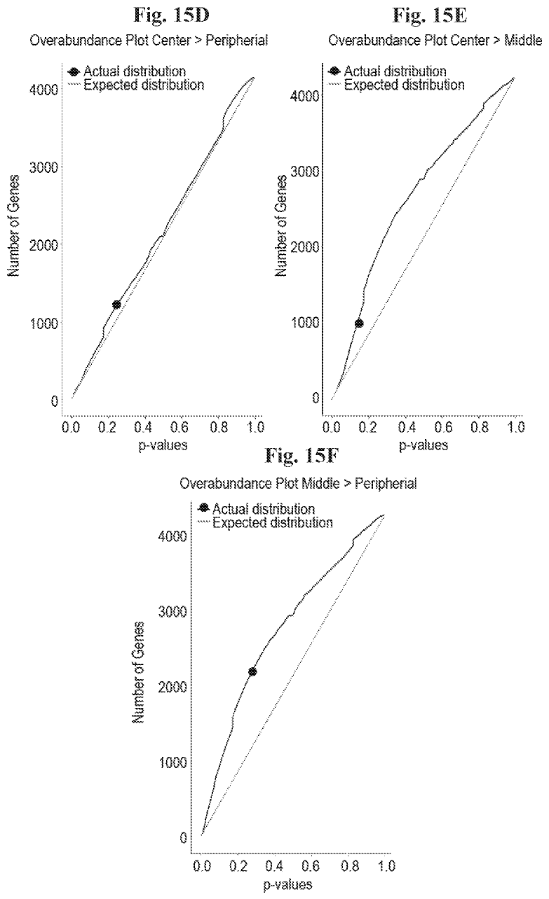

In order to target the heterogeneity of a single tumor, the proteins probed from three different positions of the same tumor in five animals were analyzed for differential expression followed by gene ontology analysis. Gene ontology analysis for center and peripheral showed that killing of cells of other organisms (p-value: 3.89E−7) were more active in center along with cell migration (p-value: 7.22E⁷) and carbohydrate metabolic process (p-value: 2.24E−7) (FIG. 15A). Analysis by function revealed structural molecule activity elevated in center of tumor compared to the peripheral area (p-value: 5.8E−7). There was a difference in cortical cytoskeleton (p-value: 8.49E−7) and cytosolic large ribosomal unit (p-value: 1.55E−8) on the basis of components.

In the process of revelations of intratumor heterogeneity comparison of center and middle regions of a tumor, genes for translation by process were higher expressed in center (FIG. 15B) (p-value: 1.01E−9). Analysis by function revealed difference in genes for translation initiation factor activity (p-value: 2.54E−9). Similar to center versus peripheral analysis by component, revealed cytosolic large ribosomal subunit is different in center versus middle (p-value: 5.85E−10).

Analyzing middle and peripheral regions of the tumor showed that the process of signal transduction was differentially expressed in middle of the tumor (FIG. 15C) (p-value: 1.98E−7). Whereas, in middle of the tumor compared to peripheral region there was a difference in genes for GTPase activity (p-value: 1.34E−7) and enzyme binding capacity (p-value: 1.3E−7). Gene ontology analysis by component revealed differences in intracellular part (p-value: 8.88E−7) and glutamatergic synapse (p-value: 6.81E−7). These finding are significant (overabundance plot of differential expression is shown in FIGS. 15D-15F. Combined, the above data shows that in-vivo e-biopsy of proteins can detect 4T1 tumors heterogeneity.

### REFERENCES

Dunham, I. et al. An integrated encyclopedia of DNA elements in the human genome. Nature (2012). doi:10.1038/nature11247
Eden, E., Navon, R., Steinfeld, I., Lipson, D. & Yakhini, Z. GOrilla: A tool for discovery and visualization of enriched GO terms in ranked gene lists. BMC Bioinformatics 10, (2009).
Newman, J. C. et al. Ketogenic Diet Reduces Midlife Mortality and Improves Memory in Aging Mice. Cell Metab. (2017). doi:10.1016/j.cmet.2017.08.004
Solomon, O. et al. RNA editing by ADAR1 leads to context-dependent transcriptome-wide changes in RNA secondary structure. Nat. Commun. 8, (2017).
Tosoian, J. J. & Antonarakis, E. S. Molecular heterogeneity of localized prostate cancer: more different than alike. Transl. Cancer Res. Vol 6, Suppl. 1 (February 2017) Transl. Cancer Res. (2017). doi:10.21037/tcr.2017.02.17

1. A method for determining if a solid tissue of a subject comprises a benign or malignant tumor, or if a space occupying lesion (SOL) within said solid tissue is malignant or benign, said method comprising:
i) placing at least one electroporation-electrode within said solid tissue, or within said SOL or in proximity thereto;
ii) applying pulsed electric field (PEF) via said at least one electroporation-electrode to thereby induce permeabilization of cells of said solid tissue or said SOL, and consequently release of at least one cellular-component therefrom to an extracellular matrix between and surrounding said cells;
iii) extracting said at least one cellular-component from said extracellular matrix; and
iv) identifying/analyzing the at least one cellular-component extracted so as to identify/determine the presence and type of the tumor within said solid tissue or determine if said SOL is malignant or benign.

2. The method of claim 1, wherein step (iii) is extracting said at least one cellular-component into at least one of said at least one electroporation-electrode, and step (iv) is carried out within said electroporation-electrode.

3. (canceled)

4. (canceled)

5. The method of claim 1, wherein said PEF is characterized by pulse number, pulse duration, electric field strength, and pulse frequency, wherein (i) said pulse number is in a range of from 1 to about 10,000; (ii) said pulse duration is in a range of from about 50 ns to about 1 s; (iii) said electric field strength is in a range of from about 0.1 to about 100 kV/cm; or (iv) said pulse frequency in in a range of from 0.1 to about 10000 Hz.

6. (canceled)

7. (canceled)

8. The method of claim 1, wherein steps (ii) and (iii), and optionally step (iv), are repeated several times, each time at a different location/area within the solid tissue and/or said SOL, without removing said at least one electroporation-electrode therefrom, and wherein said at least one cellular-component that is released into said extracellular matrix at each location/area, is kept apart for separate analysis in step (iv).

9. (canceled)

10. (canceled)

11. The method of claim 1, wherein two electroporation-electrodes are used to generate said PEF between them, and wherein: both electroporation-electrodes are placed within said solid tissue, or within said SOL or in proximity thereto; or one electroporation-electrode is placed within said solid tissue, or within said SOL or in proximity thereto, and the other electroporation-electrode is positioned at a remote location on the body of said subject.

12. (canceled)

13. (canceled)

14. The method of claim 1, wherein said at least one electroporation-electrode each independently is designed to enable penetration into said solid tissue, or into said SOL or in proximity thereto, and is: (i) a hollow tube; (ii) a solid rod engulfed in a retentive tube/cannula; or (iii) a solid rod at least partially coated with an adhesive material capable of reversibly adsorbing, associating with, and/or linking at least one of said at least one cellular-component.

15. The method of claim 14, wherein said at least one electroporation-electrode is hollow, and the at least one cellular-component released to the extracellular matrix is extracted in step (iii) by suction via said at least one hollow electroporation-electrode, and wherein said method further comprises a step of inserting at least one liquid into said solid tissue, or into said SOL or in proximity thereto, via said at least one hollow electroporation-electrode, and the at least one cellular-component released to the extracellular matrix is extracted in step (iii) by suction together with said liquid via said at least one hollow electroporation-electrode.

16. (canceled)

17. The method of claim 15, wherein said at least one liquid is: (i) an aqueous solution and the at least one cellular-component released to the extracellular matrix is diluted therein for extraction; (ii) an oil and the at least one cellular-component released to the extracellular matrix is encapsulated by said oil to form a micelle that is then extracted by suction; or (iii) aqueous solution and an oil inserted sequentially in that order, such that the at least one cellular-component released to the extracellular matrix is first diluted in the aqueous solution, and then encapsulated by said oil to form a micelle that is extracted by suction.

18. (canceled)

19. (canceled)

20. The method of claim 14, wherein: said at least one electroporation-electrode is a solid rod engulfed in a retentive tube/cannula, and the at least one cellular-component released to the extracellular matrix is extracted in step (iii) by suction via said tube/cannula after extraction of the solid rod therefrom, and wherein said method further comprises a step of inserting at least one liquid into said solid tissue, or into said SOL or in proximity thereto, via said tube/cannula, and the at least one cellular-component released to the extracellular matrix is extracted in step (iii) by suction together with said liquid via said tube/cannula.

21. (canceled)

22. The method of claim 20, wherein said at least one liquid is: (i) an aqueous solution and the at least one cellular-component released to the extracellular matrix is diluted therein for extraction; (ii) an oil and the at least one cellular-component released to the extracellular matrix is encapsulated by said oil to form a micelle that is extracted by suction; or (iii) an aqueous solution and an oil inserted sequentially in that order, and the at least one cellular-component released to the extracellular matrix is first diluted in the aqueous solution and then encapsulated by said oil to form a micelle that is extracted by suction.

23. (canceled)

24. (canceled)

25. The method of claim 14, wherein said at least one electroporation-electrode is a solid rod at least partially coated with an adhesive material capable of reversibly adsorbing, associating with, and/or linking at least one of said at least one cellular-component, and the at least one cellular-component released to the extracellular matrix is analyzed/identified in step (iv) outside the subject's body after removing said at least one electroporation-electrode from the subject's body and releasing said at least one cellular-component therefrom.

26. The method of claim 1, wherein said at least one cellular-component is analyzed/identified in step (iv) by one or more methods each independently selected from protein sequencing, polymerase chain reaction (PCR), sequencing, microarray, chromatography, and mass spectrometry, and wherein the presence and type of said tumor within said solid tissue, and/or if said SOL is malignant or benign, is determined according to at least one of said identified/analyzed cellular-components.

27. (canceled)

28. (canceled)

29. A device for the extraction of at least one cellular-component from cells of a solid tissue of a subject and/or from cells of a space occupying lesion (SOL) within said solid tissue, for determining if said solid tissue comprises a benign or malignant tumor, or if said SOL is malignant or benign, said device comprising:
(i) at least one electroporation-electrode designed to be associated with an electric generator, and to generate a pulsed electric field (PEF); and
(ii) a cellular-components extraction-element,
wherein upon introducing said at least one electroporation-electrode into said solid tissue, or into said SOL or in proximity thereto, and applying a PEF, said PEF induces permeabilization of said cells and consequently said at least one cellular-component exits to an extracellular matrix between and surrounding said cells, and is then extracted by said extraction-element.

30. (canceled)

31. The device of claim 29, wherein:
(i) said at least one electroporation-electrode comprises, or is associated with, a tissue-penetrating element; and/or
(ii) said device comprises: (a) a single electroporation-electrode that comprises a support-element with first- and second electrical-conductors mounted thereon for generating PEF within said solid tissue, or said SOL or in proximity thereto; or (b) two separate electroporation-electrodes, each comprising a support-element with an electrical-conductor mounted thereon for generating PEF within said solid tissue, or said SOL or in proximity thereto.

32.-35. (canceled)

36. The device of claim 29, wherein said extraction-element is an adhesive material capable of reversibly adsorbing, associating with, and/or linking at least one of said at least one cellular-component, wherein said support-element is at least partially coated with said adhesive material.

37. The device of claim 29, further comprising, or is associated with, a suction unit, and wherein:
(i) said at least one electroporation-electrode or said support-element is hollow, and constitutes said extraction-element through which said at least one cellular-component can be extracted by suction; or
(ii) said extraction-element is a retentive tube/cannula engulfing said support-element, such that after PEF is completed and said at least one electroporation-electrode is withdrawn from within said tube/cannula, the at least one cellular-component can be extracted from the extracellular matrix by suction via said tube/cannula.

38. (canceled)

39. (canceled)

40. The device of claim 37, wherein said device is associated or designed to be associated with a liquid reservoir and a pump, for inserting/pumping at least one liquid into said solid tissue and/or said SOL via said support-element for diluting said at least one cellular-component released to the extracellular matrix, such that it can be extracted by suction together with said liquid via said extraction-element.

41. The device of claim 37, further comprising a closure-element designed to allow or prevent passage of liquids via said hollow electroporation-electrode or said tube/cannula.

42.-44. (canceled)
